(19) United States
(12) Patent Application Publication
(10) Pub. No.: US 2025/0326731 A1
(43) Pub. Date: Oct. 23, 2025

(54) CATALYST-CONTROLLED SITE-SELECTIVE METHYLENE C-H LACTONIZATION OF DICARBOXYLIC ACIDS

(71) Applicant: THE SCRIPPS RESEARCH INSTITUTE, La Jolla, CA (US)

(72) Inventors: Jin-Quan YU, San Diego, CA (US); Hau Sun Sam CHAN, San Diego, CA (US)

(21) Appl. No.: 18/864,034

(22) PCT Filed: May 8, 2023
(86) PCT No.: PCT/US2023/066723

§ 371 Date: Nov. 8, 2024

Related U.S. Application Data
(60) Provisional application No. 63/339,627, filed on May 9, 2022.

(51) Int. Cl. C07D 307/935; B01J 31/18; B01J 31/22; C07D 307/33; C07D 307/83; C07D 307/88; C07D 307/94; C07D 309/30; C07D 311/76; C07D 311/92; C07D 405/04; C07D 493/04; C07D 493/10; C07D 495/04

(52) U.S. Cl. CPC C07D 307/935; B01J 31/181; B01J 31/2239; C07D 307/33; C07D 307/83; C07D 307/88; C07D 307/94; C07D 309/30; C07D 311/76; C07D 311/92; C07D 405/04; C07D 493/04; C07D 493/10; C07D 495/04; B01J 2231/49; B01J 2531/004; B01J 2531/824

(57) ABSTRACT

Disclosed herein is the catalyst-controlled site-selective activation of β- and γ-methylene C—H bonds of free carboxylic acids which heretofore was unknown and has remained a tremendous challenge. Described herein in the enablement of such chemical reactivity with ubiquitous dicarboxylic acids which possess inert, methylene-rich backbones and dual functional groups which opened up pathways for the construction of complex molecular scaffolds for organic synthesis. Herein we show that with a pair of palladium catalysts, it is possible to perform highly site-selective monolactonization reactions with a wide range of dicarboxylic acids, generating topologically diverse and synthetically useful γ- and δ-lactones via site-selective β- or γ-methylene C—H activation. The remaining carboxyl group serves as a versatile linchpin for further synthetic applications as demonstrated by the total synthesis of two natural products, myrotheciumone A and pedi cellosine, from abundant dicarboxylic acids.

### CROSS REFERENCE TO RELATED APPLICATION

This application claims priority to U.S. provisional patent application No. 63/339,627, which was filed on May 9, 2022, and which is hereby incorporated by reference in its entirety.

### GOVERNMENT SUPPORT

This invention was made with government support under grant number GM084019 awarded by the National Institutes of Health. The government has certain rights in the invention.

### FIELD OF THE INVENTION

A pair of palladium catalysts have been identified and used to enable site-selective activation of β- and γ-methylene C—H bonds for the synthesis of γ- and δ-lactones from abundant dicarboxylic acids.

### BACKGROUND OF THE INVENTION

Achieving site-selective methylene C—H activation of aliphatic carboxylic acids is a significant challenge in organic synthesis. The freedom to site-selectively functionalize ubiquitous molecular backbones with multiple methylene C—H bonds would enable rapid construction of a great variety of molecular scaffolds, and usher in novel retrosynthetic thinking that was previously deemed implausible. Although directed C—H activation of aliphatic carboxylic acids has been shown possible with platinum (1, 2) and palladium catalysis (3-11), development of catalysts for the activation of methylene C—H bonds is nascent, and methods for distinguishing between the C—H bonds of multiple methylene units that are similar to one another is unknown. Recent breakthroughs in ligand development have provided a glimmer of hope for the palladium-catalyzed, carboxylic acid-directed activation of β-methylene C—H/bonds, culminating in the reports of a dehydrogenation reaction and a deuteration reaction (12, 13). However, no other C-heteroatom bond formation reaction nor activation of the γ-methylene C—H bond are hitherto known using this approach. Alternative strategies for the functionalization of methylene C—H bonds of free carboxylic acids rely on high-valent, electrophilic Fe or Mn catalysts that operate via formation of metal-oxo intermediates (14-16). Yet, catalyst-controlled site-selectivity between adjacent methylene units remain unconquered.

Thus, there is a need in the field for the development of a method to enable site-selective activation of β- and γ-methylene C—H bonds for the synthesis of γ- and δ-lactones from abundant dicarboxylic acids.

### SUMMARY OF THE INVENTION

Disclosed herein is the catalyst-controlled site-selective activation of β- and γ-methylene C—H bonds of free carboxylic acids which heretofore was unknown and has remained a tremendous challenge. Described herein in the enablement of such chemical reactivity with ubiquitous dicarboxylic acids which possess inert, methylene-rich backbones and dual functional groups which opened up pathways for the construction of complex molecular scaffolds for organic synthesis. Herein we show that with a pair of palladium catalysts, it is possible to perform highly site-selective monolactonization reactions with a wide range of dicarboxylic acids, generating topologically diverse and synthetically useful γ- and δ-lactones via site-selective β- or γ-methylene C—H activation. The remaining carboxyl group serves as a versatile linchpin for further synthetic applications as demonstrated by the total synthesis of two natural products, myrotheciumone A and pedicellosine, from abundant dicarboxylic acids.

As disclosed herein, the application provides a method of γ- or δ-lactonization via β-C—H activation, comprising i) treating a dicarboxylic acid substrate with a quinoline-pyridone or pyridine-pyridone ligand in the presence of a Pd source; and ii) addition of p-xyloquinone (BQ3), an Ag salt, and K₂HPO₄ in a reaction vessel.

The application further provides the above method, wherein the quinoline-pyridone or pyridine-pyridone ligand is selected from the group consisting of:

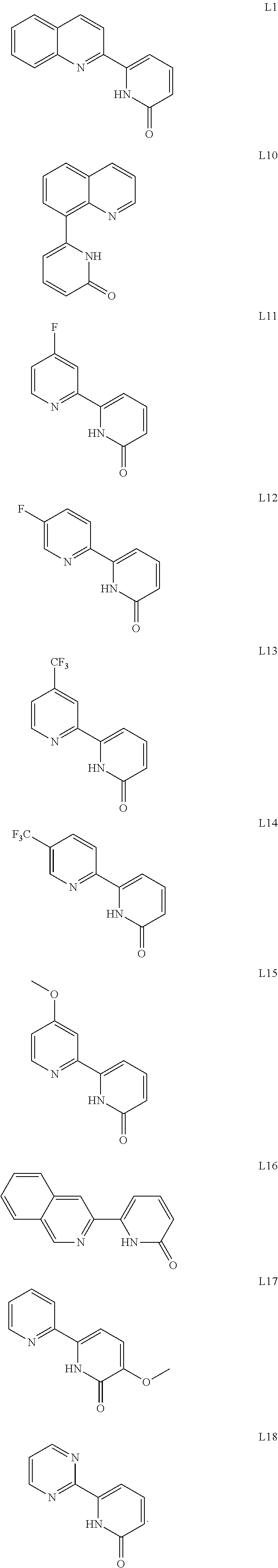

The application further provides the above method of γ-C—H lactonization via β-C—H activation comprising i) treating a dicarboxylic acid substrate with a quinoline-pyridone or pyridine-pyridone ligand in the presence of a Pd source; and ii) addition of p-xyloquinone (BQ3), an Ag salt, and K₂HPO₄ in a reaction vessel, wherein the dicarboxylic acid substrate is 1.0 eq. adipic acid, the quinoline-pyridone ligand is 12 mol % L1, the Pd source is 10 mol % Pd(OAC)₂, the Ag salt is 2.0 eq. Ag₂CO₃, with 2.0 eq. BQ3, 1.0 eq. K₂H—PO₄ in HFIP at 100° C. for 36 h and the reaction vessel is an 8-10 mL vial.

The application further provides the above method of δ-lactonization via β-C—H activation comprising i) treating a dicarboxylic acid substrate with a quinoline-pyridone or pyridine-pyridone ligand in the presence of a Pd source; and ii) addition of p-xyloquinone (BQ3), an Ag salt, and K₂HPO₄ in a reaction vessel, wherein the dicarboxylic acid substrate is 1.0 eq. pimelic acid, the quinoline-pyridone ligand is 12 mol % L1, the Pd source is 10 mol % Pd(OAC)₂, the Ag salt is 2.0 eq. Ag₂CO₃, with 2.0 eq. BQ3, 1.0 eq. K₂HPO₄ in HFIP at 100° C. for 36 h and the reaction vessel is an 8-10 mL vial.

### BRIEF DESCRIPTION OF THE FIGURES

FIG. 1 Lactonization via activation of methylene C—H bonds (A) Natural products with lactonization at methylene carbons (B) Two major challenges for site-selective C—H lactonization of dicarboxylic acids (C) Three modes of lactonization by ligand-control (D) Application of the lactonization methodology to the total syntheses of pedicellosine and myrotheciumone A.

FIG. 2 Substrate scope of γ- and δ-lactones from β-C—H activation. (A) γ-Lactonization via β-C—H activation. (B) δ-Lactonization via β-C—H activation. Reaction conditions: Substrate (0.1 mmol), Pd(OAc)₂ 10 mol %, Ligand L1 12 mol %, Ag₂CO₃ (2.0 eq.), BQ3 (2.0 eq.), K₂HPO₄ (0.35 eq.), CsOAc (0.4 eq.), HFIP (1.0 mL), 100° C., 36 h. Isolated yields are reported. †Isolated as the corresponding benzyl ester.

FIG. 3 Substrate scope of γ-lactones from γ-C—H activation, demonstration of switchable site-selectivity, and examples of silver-free lactonization reactions with MnO₂ as the oxidant. (A) γ-Lactonization via γ-C—H activation. Reaction conditions: Substrate (0.1 mmol), Pd(OAc)₂ 10 mol %, Ligand L2 12 mol %, Ag₂CO₃ (2.0 eq.), BQ3 (2.0 eq.), K₂HPO₄ (0.35 eq.), CsOAc (0.4 eq.), HFIP (1.0 mL), 100° C., 36 h. Isolated yields are reported. †Isolated as the corresponding benzyl ester. ‡Based on reactive diastereomer (see The Experimental section for detailed analysis). (B) Demonstration of switchable site-selectivity with 19 examples. (C) Examples of silver-free lactonization with MnO₂ as the oxidant (22 examples). Reaction conditions: Substrate (0.1 mmol), Pd(OAc)₂ 10 mol %, Ligand L1 or L2 12 mol %, MnO₂ (4.0 eq.), BQ3 (2.0 eq.), K₂HPO₄:KH₂PO₄:CsOAc (1.0:1.5:1.0, 0.75 eq. in 162 total), HFIP (1.0 mL), 100° C., 36 h. \hat{ }NMR yields.

FIG. 4 Total synthesis of Myrotheciumone A and Pedicellosine. (A) Total synthesis of Myrotheciumone A. (1) MeI (2.0 eq.), K₂CO₃ (3.0 eq.), acetone, reflux, 3 hr. (2) Ph₃PCH₃Br (2.9 eq.), tBuOK (2.4 eq.), toluene, r.t., overnight. (3) mCPBA (1.2 eq.), CH₂Cl₂, r.t., overnight. (4) TMSOTf (2.0 eq.), 2,6-lutidine (2.0 eq.), toluene, −78° C. to r.t., overnight. (5) cat. Pivalic acid (10 mol %), triethyl orthoacetate, neat, 155° C., overnight. then cat. p-TsOH (10 mol %), toluene, reflux, overnight. (6) cat. PtO₂, H₂ (4-layered balloon), AcOH, r.t., overnight. (7) 15% aq. NaOH, reflux, overnight. (8) Pd(OAc)₂ (10 mol %), Ligand L2 (12 mol %), BQ3 (2.0 eq.), Ag₂CO₃ (2.0 eq.), K₂HPO₄ (0.35 eq.), CsOAc (0.40 eq.), HFIP, 100° C., 36 h. (9) (Ir[dF(CF₃)ppy]₂(dtbpy))PF₆ (1 mol %), Cs₂CO₃ (1.5 eq.), NaBH₄ (1.2 eq.), O₂ atmosphere, CH₂Cl₂, 40° C., 4×100 W Blue LED lamps, 40 h. †Yields based on reactive diastereomers of 59 (see The Experimental section for detailed analysis). (B) Total synthesis of Pedicellosine. (1) Pd(OAc)₂ (10 mol %), Ligand L2 (12 mol %), BQ3 (2.0 eq.), Ag₂CO₃ (2.0 eq.), K₂HPO₄ (0.35 eq.), CsOAc (0.40 eq.), HFIP, 100° C., 36 h. (2) BH₃·Me₂S (1.6 eq.), THF, 0° C. to r.t., overnight. (3) EDCI (1.5 eq.), DMAP (20 mol %), 2,3-dihydroxybenzoic acid (1.5 eq.), CH₂Cl₂, r.t., overnight.

### DETAILED DESCRIPTION OF THE INVENTION

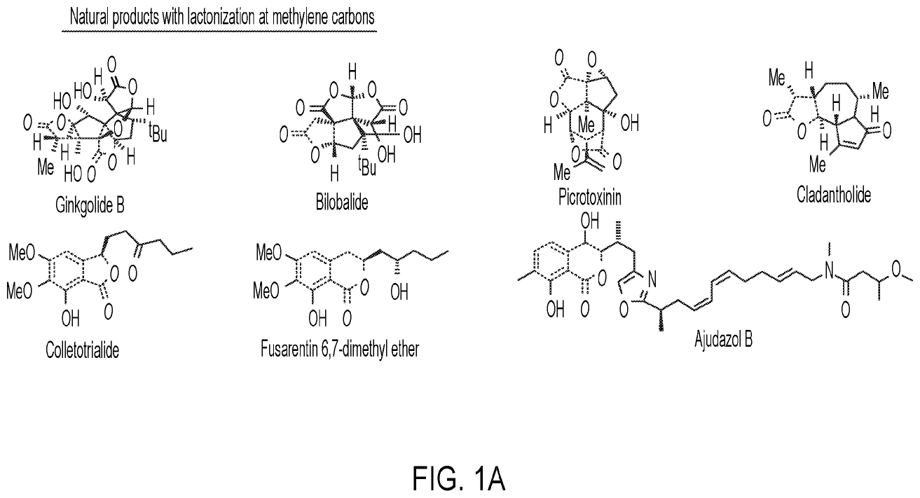

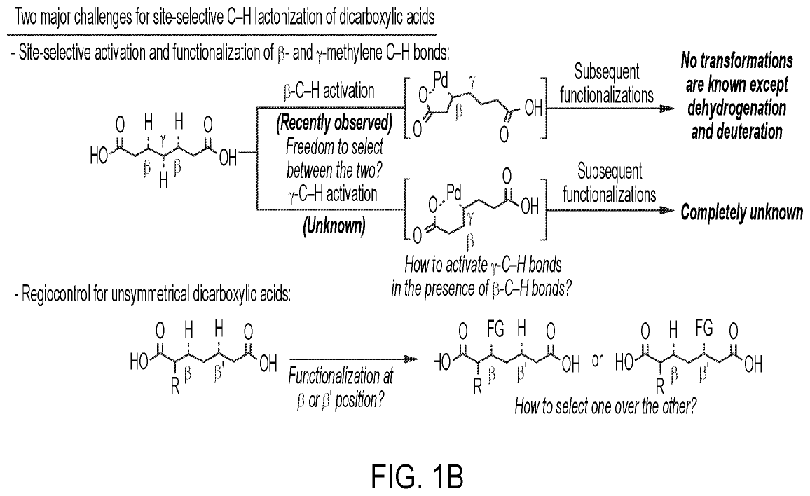

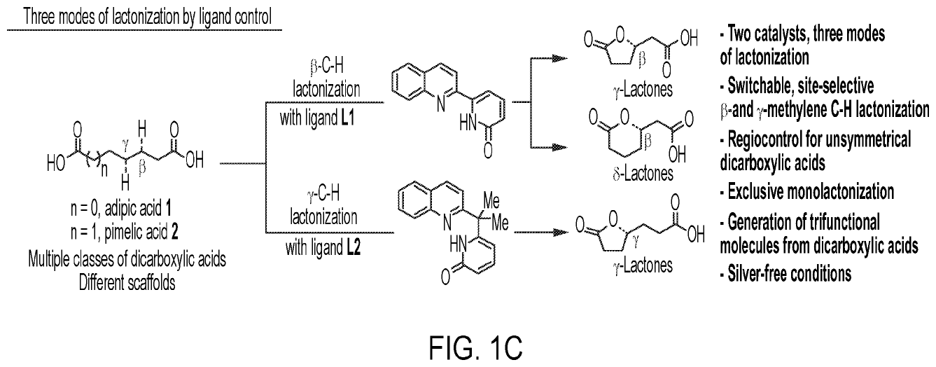

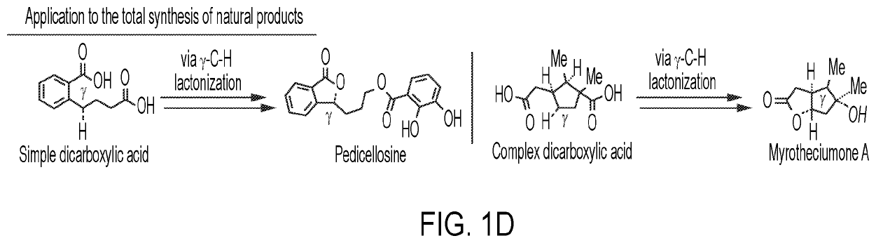

Disclosed herein is the development of site-selective C—O bond formation reactions of dicarboxylic acids through lactonization, which was driven by the fact that lactones are ubiquitous in organic chemistry, and that the monolactonization of dicarboxylic acids would leave the remaining carboxyl group as a versatile handle for further synthetic elaborations (17). It was estimated that more than 3,000 γ-lactones exist in nature (18, 19), and the formation of lactones at methylene carbons is commonplace in natural products, as demonstrated in the structures of ginkgolide B, bilobalide, picrotoxinin, and cladantholide (20, 21). Benzo-fused lactones such as phthalides and isochromanones are also recurring structures in bioactive natural products, such as colletotrialide, fusarentin 6,7-dimethyl ether, and ajudazol B (22, 23) (FIG. 1a). The principal challenge for the development of the desired lactonization reactions has been to achieve site-selective activation of either β- or γ-methylene C—H bonds by catalyst design, and thus enable the freedom to functionalize the methylene backbone of dicarboxylic acids at multiple positions. Another significant challenge is to realize regiocontrol for unsymmetrical dicarboxylic acids, thereby prohibiting the unselective generation of mixtures of regioisomeric products (24) (FIG. 1b).

Herein disclosed are two distinct catalysts for site-selective β- and γ-C—H activation of dicarboxylic acids using quinoline-pyridone ligands, which lead to three modes of lactonization for the synthesis of γ- and δ-lactones from various dicarboxylic acids (FIG. 1c). Symmetrical dicarboxylic acids such as adipic acid and pimelic acid are thus converted into unsymmetrical lactone acids using this methodology. Unsymmetrical dicarboxylic acids generally provide lactone acids where the more substituted fraction of the molecule preferentially lactonizes. These lactonization reactions convert dicarboxylic acids into trifunctional molecules with three orthogonal sites of reactivity that are amenable to chemoselective transformations for complex molecule synthesis. It was also discovered that the use of inexpensive and abundant MnO₂ as the oxidant is compatible with the lactonization reactions, demonstrating the possibility of using silver-free conditions for palladium-catalyzed, carboxylic acid-directed lactonization of methylene C—H bonds. The utility of this methodology was demonstrated with two total syntheses, affording the natural products pedicellosine and myrotheciumone A from dicarboxylic acids (FIG. 1d).

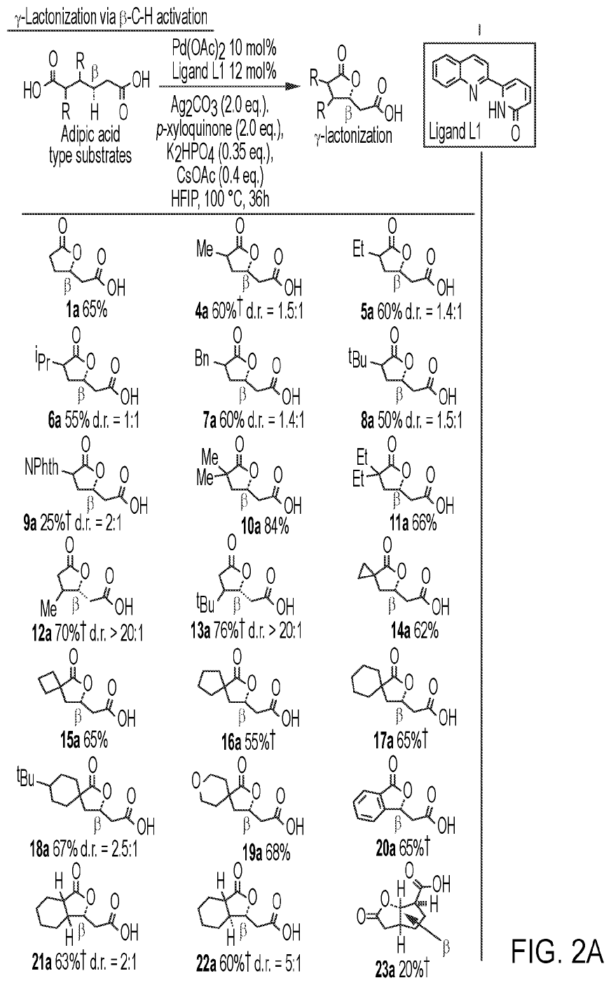

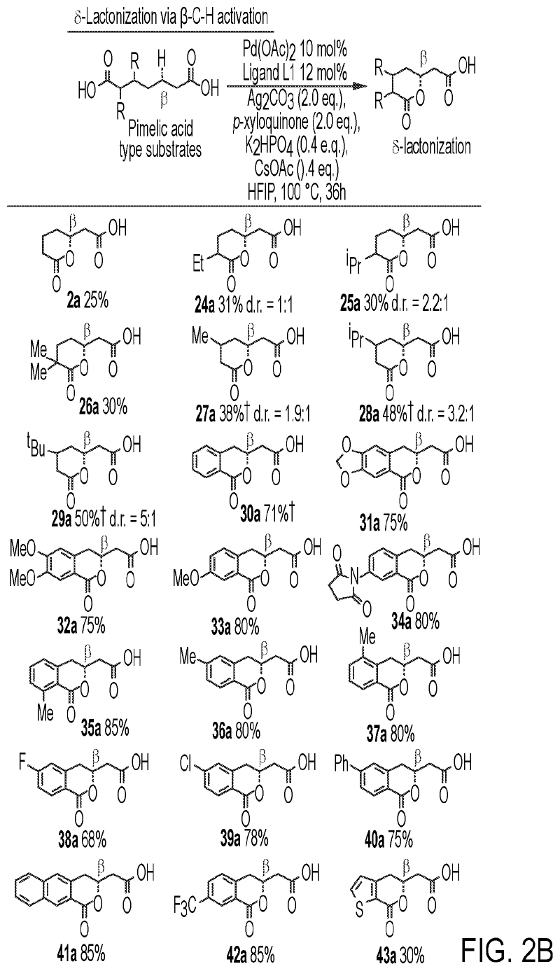

The development of the lactonization reaction began with exploratory studies using adipic acid 1 as the standard substrate. It was realized that bis-carboxylate chelation of the dicarboxylic acid could prevent the desired directed C—H activation, and thus a highly efficient bidentate ligand would be required to overcome this potential hurdle. Building on the precedent that pyridine-pyridone type ligands could promote β-methylene C—H activation in a palladium-catalyzed, carboxylic acid-directed dehydrogenation reaction (13), a series of pyridine-pyridone type ligands were investigated for the desired C—O bond forming reactivity (see The Experimental section). Following extensive optimization of reaction parameters, the five-membered chelating quinoline-pyridone ligand L1 emerged as the most effective at initiating the lactonization of 1 to provide γ-lactone 1a in 65% isolated yield (FIG. 2a, first product). The formation of γ-lactone 1a from adipic acid 1 implied two mechanistic possibilities: γ-lactonization via γ-C—H activation or γ-lactonization via β-C—H activation. In other words, the observed γ-lactonization could occur via directed γ-C—H activation followed by γ-lactonization by the same carboxyl group, or it could occur via directed β-C—H activation by one of the carboxyl groups followed by γ-lactonization with the remaining carboxyl group. To distinguish between these two possibilities, a control experiment using pimelic acid 2 as the substrate was carried out. It was found that 6-lactone 2a was generated in this experiment in 25% isolated yield (FIG. 2b, first product). This observation suggests carboxylic acid-directed β-C—H activation with ligand L1 is more probable, which is then followed by δ-lactonization using the carboxylic acid at the other end of the molecule. The other possibility of δ-lactonization, which is via a δ-C—H activation pathway, is deemed highly unlikely. Consistent with this hypothesis, the mono methyl ester of adipic acid 1, monomethyl adipate 3, was found to be unreactive for γ-lactonization, supporting the conclusion that one carboxylic acid is responsible for directed β-C—H activation, and the other carboxylic acid is responsible for γ-lactonization.

The generality of these two modes of lactonization via β-C—H activation was investigated with a series of dicarboxylic acids with various substitutions and topologies. For γ-lactonization reactions via β-C—H activation with ligand L1 (FIG. 2a), substrates possessing α-hydrogens and without Thrope-Ingold assistance that were traditionally found to be less reactive all provided the desired products in synthetically useful yields (5). The α-mono substituted products with increasing steric demand of the substitution (Me 4a, Et 5a, iPr 6a, Bn 7a, tBu 8a) were isolated in 50-60% yield. The product 9a with a NPhth substitution at its α-position, however, only provided a 25% isolated yield as the benzyl ester. For the product 10a that bears the gem-dimethyl substitution at its α-position, it was isolated in 84% yield. Increasing the size of the substitution to a gem-diethyl system, as for product 11a, resulted in a decrease in isolated yield to 66%. Products 12a and 13a possessing substitution at their β-position but with fully unsubstituted α-positions displayed high reactivity and diastereoselectivity, giving isolated yields around 70% as their benzyl esters and favoring the anti-isomer with a d.r.>20:1. Spirocyclic structures at the α-position were also forged with ease, providing lactones possessing spirocyclic cyclopropyl (14a), cyclobutyl (15a), cyclopentyl (16a), cyclohexyl (17a, 18a), and 4-tetrahydropyranyl (19a) systems in 55-68% yield. Fused structures were also found to be well tolerated for this lactonization methodology, providing benzofused product 20a in 65% isolated yield as its benzyl ester, and fused 5,6-bicyclic systems with a cis (21a) and a trans (22a) junction in 63% and 60% yield as their benzyl esters, respectively. The more strained 5,5-bicyclic system 23a with endocyclic β-methylene C—H bonds was also amenable to synthesis. Yet, it was only isolated in 20% yield as its benzyl ester.

For 6-lactonization reactions via β-C—H activation (FIG. 2b), aliphatic systems were challenging substrates (2a, 24a-29a), in which substitution at the β-position was required to elevate the isolated yield to 38-50% (27a-29a). However, the performance of the reaction for the synthesis of benzo-fused products was superior, providing the products 30a-43a in 68-85% yield, tolerating a range of aromatic substitutions such as oxygen (31a-33a), nitrogen (34a), alkyl (35a-37a, 40a), halide (38a-39a) and a trifluoromethyl group (42a). Modifying the identity of the aromatic ring was also possible, as demonstrated by the formation of naphthalene-fused system 41a and the thiophene-fused system 43a, albeit the latter was only isolated in 30% yield.

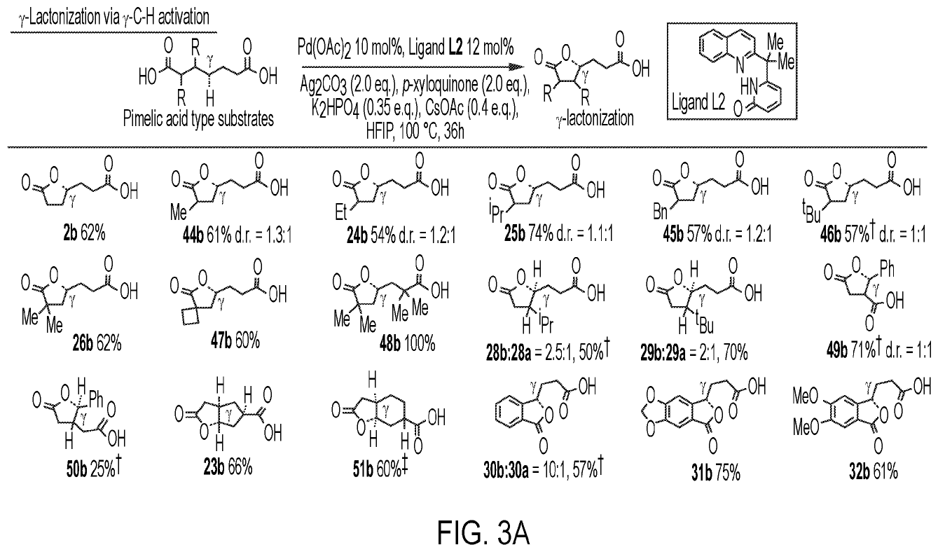

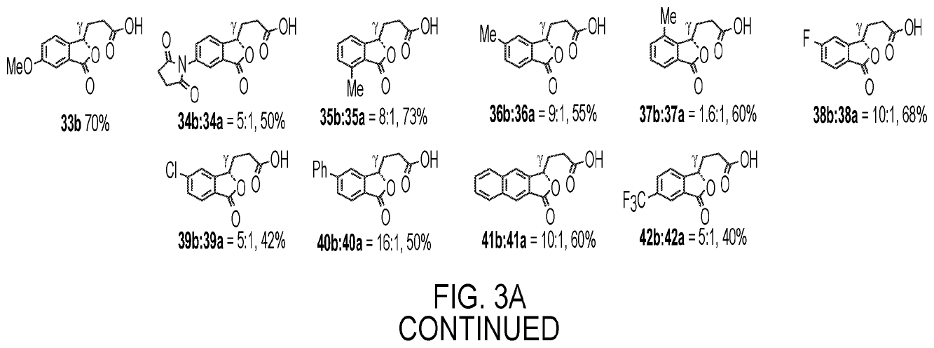

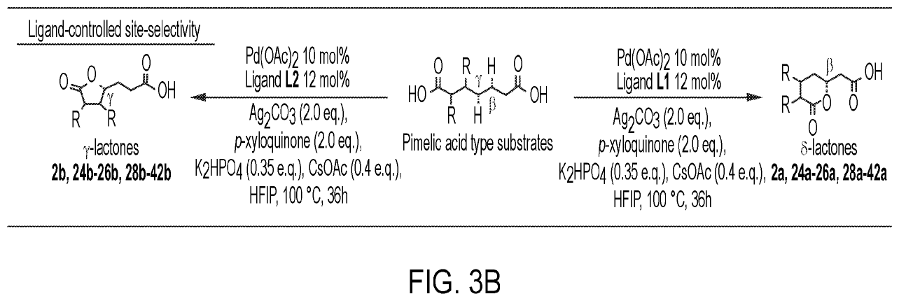

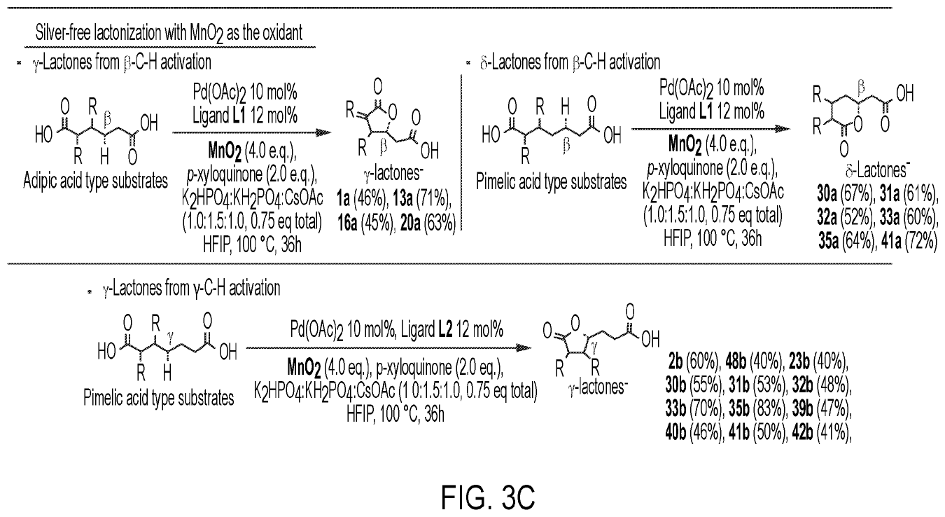

Subsequently, whether the 7-methylene C—H bonds of pimelic acid 2 could also be activated for γ-lactonization was investigated. This was believed to present a significant challenge for C—H activation because activation of the competing β-methylene C—H bonds that would result in five-membered cyclopalladation was expected to be both kinetically and thermodynamically preferred (25). It was expected that overcoming this hurdle for C—H activation could lead to two distinct site-selective lactonization pathways via β- or γ-methylene C—H activation. With this objective in mind, other types of quinoline-pyridone ligands were explored. It was discovered that ligand L2 provided the desired γ-lactone 2b in 62% isolated yield (FIG. 3a). For this mode of lactonization via γ-C—H activation, traditionally less reactive substrates possessing α-hydrogens, and without Thorpe-Ingold assistance, all provided the desired products in synthetically useful yields (5). The α-mono substituted products with increasing steric demand of the substitution patterns (Me 44b, Et 24b, iPr 25b, Bn 45b, tBu 46b) were isolated in 54-74% yield. Of the products that were quaternized at their α-positions, the product 26b with single gem-dimethyl substitution was isolated in 62% yield. The product 48b with double gem-dimethyl substitutions, the isolated yield was increased to 100%. The formation of spirocyclic systems were also tested with this lactonization methodology, however, only the cyclobutyl system was amenable to lactonization, giving 47b in an isolated yield of 60%. γ-Lactones 28b and 29b possessing substitution at their β-position but with fully unsubstituted α-positions were isolated as the anti-diastereomers along with the minor products δ-lactones 28a and 29a in ca. 2:1 ratio, with a total yield of 50-70%. Dicarboxylic acids that possess molecular scaffolds distinct from linear adipic and pimelic acid variants were also investigated. Interestingly, the product 49b arising from α-benzyl succinic acid was produced only with the use of ligand L2 but not with ligand L1, suggesting a γ-C—H activation pathway was likely for its lactonization. It was isolated in 70% yield after conversion to its benzyl ester. The product 50b, arising from β-benzyl glutaric acid, was isolated as its benzyl ester in 25% yield as a single anti-diastereomer. Substrates with endocyclic γ-methylene C—H bonds were also found to be viable for this lactonization protocol, as exemplified with products 23b and 51b, generating lactones with fused 5,5-bicyclic and fused 5,6-bicyclic systems in good yields as single diastereomers. Benzo-fused γ-lactones (30b-42b) were also afforded in 40-75% total yields with predominant (γ:β>8:1) site-selectivity at the γ-position (26, 27), except for products 34b, 37b, 39b, and 42b in which the site-selectivity was found to be lower. The collective synthesis of a series of γ-lactones (2b, 24b-26b, 28b-42b) and δ-lactones (2a, 24a-26a, 28a-42a) from the same substrates elegantly demonstrated the desired ligand-controlled switchable site-selectivity for carboxylic acid directed, β-methylene and γ-methylene C—H lactonizations (FIG. 3b).

With the scope of the lactonization established, silver-free conditions for the three modes of lactonization reactions were sought and MnO₂ was found to be a viable replacement for Ag₂CO₃ as the oxidant. Upon further fine-tuning of the reaction conditions (see The Experimental section), the lactonization reactions could be rendered silver-free across a series of substrates (FIG. 3c).

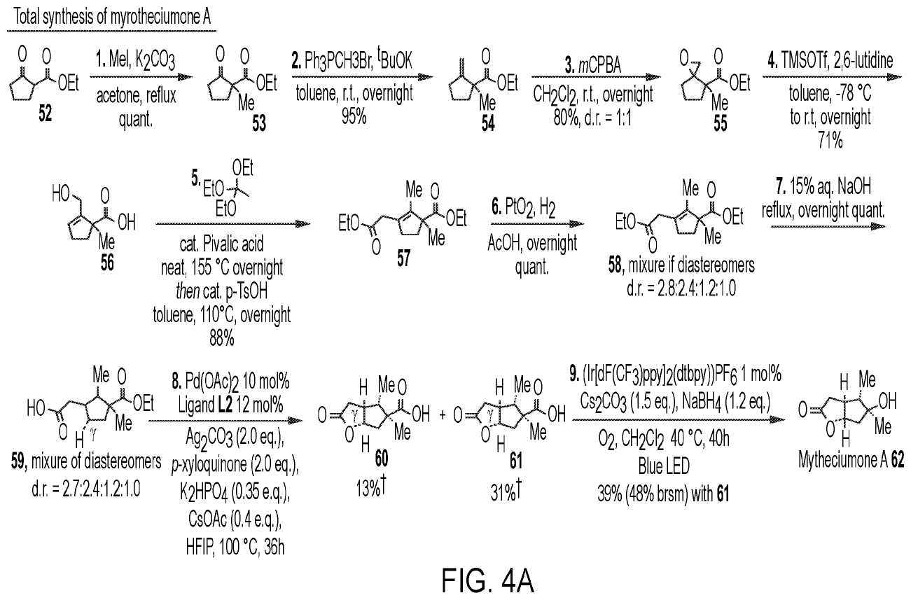

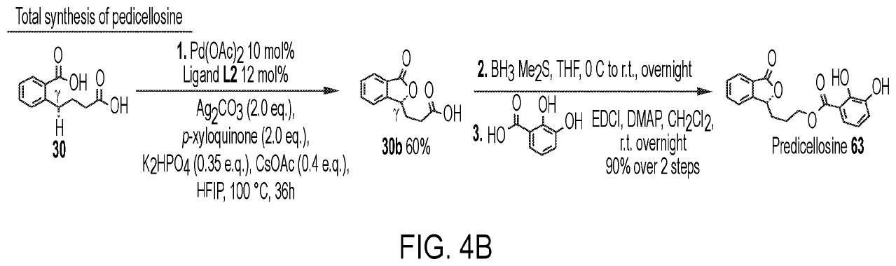

In light of the broad scope of lactones prepared by this methodology, two total syntheses as a means to demonstrate the synthetic utility offered by the fusion of dicarboxylic acids and C—H lactonization (FIG. 4) were attempted. Myrotheciumone A 62 (28), a bicyclic cytotoxic lactone isolated from the fungus Ajuga decumbens, possesses the bicyclic 5,5-fused scaffold as in lactone 23b (FIG. 3a). Retrosynthetic disconnection at the lactone C—O bond indicated that complex dicarboxylic acid 59 would be the synthetic intermediate for the key C—H lactonization step. This intermediate 59 was prepared efficiently, albeit as a diastereomeric mixture, from commercially available ethyl 2-oxocyclopentane-1-carboxylate 52 in 7 steps. Thus, ethyl 2-oxocyclopentane-1-carboxylate 52 was methylated with MeI in refluxing acetone to provide 53 in quantitative yield, followed by a Wittig reaction with Ph₃PCH₃Br to give terminal alkene 54 in 95% yield. The terminal alkene 54 was epoxidized with mCPBA to give an inconsequential 1:1 diastereomeric mixture of epoxides 55 in 80% yield, which were isomerized with TMSOTf and 2,6-lutidine in toluene (−78° C. to room temperature, overnight) to give allylic alcohol 56 in 71% yield. The allylic alcohol 56 underwent a Johnson-Claisen rearrangement, followed by olefin isomerization with p-TsOH in refluxing toluene to give the complex cyclopentene 57 in 88% yield. The cyclopentene 57 was hydrogenated with cat. PtO₂ in AcOH under the H₂ pressure of a 4-layered balloon overnight to provide the complex cyclopentane 58 in quantitative yield as a diastereomeric mixture. Subsequent basic ester hydrolysis of 58 with 15% aq. NaOH at reflux provided the dicarboxylic acid 59 in quantitative yield. The γ-lactonization of dicarboxylic acid 59 provided the desired lactone 61 in 31% yield, and its diastereomer 60 in 13% yield. The yields of the lactones 60 and 61 were calculated based on the reactive diastereomers of 59 (see the Experimental section for detailed analysis). The last step of the synthesis was a photocatalytic decarboxylative hydroxylation of 61 which provided the natural product myrotheciumone A 62 in 39% yield (48% yield based on recovered starting material) (29). The total synthesis was completed in a total of 9 steps from ethyl 2-oxocyclopentane-1-carboxylate (30). Pedicellosine 63 (31), a phthalide isolated from the leaves of Gentiana pedicellate, possesses the benzo-fused lactone structure as in lactone 30b (FIG. 3a). The synthesis began with the preparation of lactone 30b followed by the reduction of the carboxylic acid to the alcohol using BH₃·Me₂S. The crude alcohol was esterified with 2,3-dihydroxybenzoic acid using EDCI as the coupling reagent, providing the natural product pedicellosine 63 in 90% yield over two steps from the lactone 30b. These two representative total syntheses demonstrate that the fusion of dicarboxylic acids and C—H lactonization constitutes a viable synthetic strategy amenable for the preparation of molecular targets of varying complexities at both late- and early-stages of synthesis.

It was concluded that site-selective β- and γ-methylene C—H activation of aliphatic acids with two quinoline-pyridone ligands L1 and L2 had been achieved. This led to the development of three modes of lactonization reaction for the syntheses of γ- and δ-lactones from a great variety of dicarboxylic acids. The utility of the methodology is demonstrated by the total syntheses of two natural products as disclosed herein.

### REFERENCES

1. L.-C. Kao, A. Sen, Platinum(II) Catalysed Selective Remote Oxidation of Unactivated C—H Bonds in Aliphatic Carboxylic Acids. J. Chem. Soc., Chem. Commun., 1242-1243 (1991).
2. B. D. Dangel, J. A. Johnson, D. Sames, Selective functionalization of amino acids in water: A synthetic method via catalytic C—H bond activation. J. Am. Chem. Soc. 123, 8149-8150 (2001).
3. R. Giri, N. Maugel, J. J. Li, D. H. Wang, S. P. Breazzano, L. B. Saunders, J. Q. Yu, Palladium-catalyzed methylation and arylation of sp² and sp³ C—H bonds in simple carboxylic acids. J. Am. Chem. Soc. 129, 3510-3511 (2007).
4. Z. Zhuang, C. bin Yu, G. Chen, Q. F. Wu, Y. Hsiao, C. L. Joe, J. X. Qiao, M. A. Poss, J. Q. Yu, Ligand-Enabled O—C(sp³)-H Olefination of Free Carboxylic Acids. J. Am. Chem. Soc. 140, 10363-10367 (2018).
5. Z. Zhuang, A. N. Herron, Z. Fan, J. Q. Yu, Ligand-Enabled Monoselective β-C(sp³)-H Acyloxylation of Free Carboxylic Acids Using a Practical Oxidant. J. Am. Chem. Soc. 142, 6769-6776 (2020).
6. Z. Zhuang, J. Q. Yu, Lactonization as a general route to β-C(sp³)-H functionalization. Nature. 577, 656-659 (2020).
7. Z. Zhuang, A. N. Herron, J. Q. Yu, Synthesis of Cyclic Anhydrides via Ligand-Enabled C—H Carbonylation of Simple Aliphatic Acids. Angewandte Chemie International Edition. 60, 16382-16387 (2021).
8. K. K. Ghosh, M. van Gemmeren, Pd-Catalyzed β-C(sp³)-H Arylation of Propionic Acid and Related Aliphatic Acids. Chemistry—A European Journal. 23, 17697-17700 (2017).
9. K. K. Ghosh, A. Uttry, A. Koldemir, M. Ong, M. van Gemmeren, Direct β-C(sp³)-H acetoxylation of aliphatic carboxylic acids. Organic Letters. 21, 7154-7157 (2019).
10. K. K. Ghosh, A. Uttry, F. Ghiringhelli, A. Mondal, M. van Gemmeren, Ligand-Enabled γ-C(sp³)-H Olefination of Free Carboxylic Acids. Angewandte Chemie International Edition. 59, 12848-12852 (2020).
11. F. Ghiringhelli, A. Uttry, K. K. Ghosh, M. van Gemmeren, Direct β- and γ-C(sp³)-H Alkynylation of Free Carboxylic Acids. Angewandte Chemie International Edition. 59, 23127-23131 (2020).
12. A. Uttry, S. Mal, M. van Gemmeren, Late-Stage β-C(sp³)-H Deuteration of Carboxylic Acids. J. Am. Chem. Soc. 143, 10895-10901 (2021).
13. Z. Wang, L. Hu, N. Chekshin, Z. Zhuang, S. Qian, J. X. Qiao, J.-Q. Yu, Ligand-controlled divergent dehydrogenative reactions of carboxylic acids via C—H activation. Science. 374, 1281-1285 (2021).
14. M. Cianfanelli, G. Olivo, M. Milan, R. J. M. Klein Gebbink, X. Ribas, M. Bietti, M. Costas, Enantioselective C—H Lactonization of Unactivated Methylenes Directed by Carboxylic Acids. J. Am. Chem. Soc. 142, 1584-1593 (2020).
15. L. Vicens, M. Bietti, M. Costas, General Access to Modified α-Amino Acids by Bioinspired Stereoselective γ-C—H Bond Lactonization. Angewandte Chemie International Edition. 60, 4740-4746 (2021).
16. M. S. Chen, M. C. White, A Predictably Selective Aliphatic C—H Oxidation Reaction for Complex Molecule Synthesis. Science. 318, 783-787 (2007).
17. J. Schwarz, B. König, Decarboxylative reactions with and without light—a comparison. Green Chemistry. 20, 323-361 (2018).
18. S. K. Sartori, M. A. N. Diaz, G. Diaz-Muñoz, Lactones: Classification, synthesis, biological activities, and industrial applications. Tetrahedron. 84, 132001-132039 (2021).
19. M. Seitz, O. Reiser, Synthetic approaches towards structurally diverse γ-butyrolactone natural-product-like compounds. Current Opinion in Chemical Biology. 9 (2005), pp. 285-292.
20. H. T. Simonsen, C. Weitzel, S. B. Christensen, in Natural Products: Phytochemistry, Botany and Metabolism of Alkaloids, Phenolics and Terpenes (Springer Berlin Heidelberg, 2013), pp. 3069-3098.
21. K. Strømgaard, K. Nakanishi, Chemistry and biology of terpene trilactones from Ginkgo biloba. Angewandte Chemie International Edition. 43, 1640-1658 (2004).
22. G. Shabir, A. Saeed, H. R. El-Seedi, Natural isocoumarins: Structural styles and biological activities, the revelations carry on. Phytochemistry. 181, 112568-112787 (2021).
23. A. D. Kinghorn, H. Falk, S. Gibbons, J. Kobayashi, Eds., Progress in the Chemistry of Organic Natural Products (Springer Nature, 2017), vol. 104.
24. F. Zaragoza Dörwald, Side Reactions in Organic Synthesis: A Guide to Successful Synthesis Design (Wiley, 2004).
25. G. Xia, J. Weng, L. Liu, P. Verma, Z. Li, J. Q. Yu, Reversing conventional site-selectivity in C(sp 3)-H bond activation. Nature Chemistry. 11, 571-577 (2019).
26. P. Novak, A. Correa, J. Gallardo-Donaire, R. Martin, Synergistic palladium-catalyzed C(sp³)-H Activation/C(sp³)-O bond formation: A direct, step-economical route to benzolactones. Angewandte Chemie International Edition. 50, 12236-12239 (2011).
27. J. M. Lee, S. Chang, Pt-Catalyzed sp³ C—H bond activation of o-alkyl substituted aromatic carboxylic acid derivatives for the formation of aryl lactones. Tetrahedron Letters. 47, 1375-1379 (2006).
28. T. Lin, G. Wang, W. Shan, D. Zeng, R. Ding, X. Jiang, D. Zhu, X. Liu, S. Yang, H. Chen, Myrotheciumones: Bicyclic cytotoxic lactones isolated from an endophytic fungus of Ajuga decumbens. Bioorganic and Medicinal Chemistry Letters. 24, 2504-2507 (2014).
S. N. Khan, M. K. Zaman, R. Li, Z. Sun, A General Method for Photocatalytic Decarboxylative Hydroxylation of Carboxylic Acids. Journal of Organic Chemistry. 85, 5019-5026 (2020).
30. S. Begum, T. K. Chakraborty, Cp2TiCl-Mediated Reductive Cyclization: Total Synthesis of Pestalotiolactone A, Myrotheciumone A, and Scabrol A. Journal of Organic Chemistry. 86, 11812-11821 (2021).
31. E. M. Mpondo, J. Garcia, A. J. Chulia, New Phthalides from Gentiana pedicellata. Planta Medica. 53, 297-298 (1987).

### EMBODIMENTS

The application provides the following embodiments:

### Embodiment 1

A method of γ- or δ-lactonization via β-C—H activation, comprising i) treating a dicarboxylic acid substrate with a quinoline-pyridone or pyridine-pyridone ligand in the presence of a Pd source; and ii) addition of p-xyloquinone (BQ3), an Ag salt, and K₂HPO₄ in a reaction vessel.

### Embodiment 2

The method of embodiment 1, wherein the quinoline-pyridone or pyridine-pyridone ligand is selected from the group consisting of:

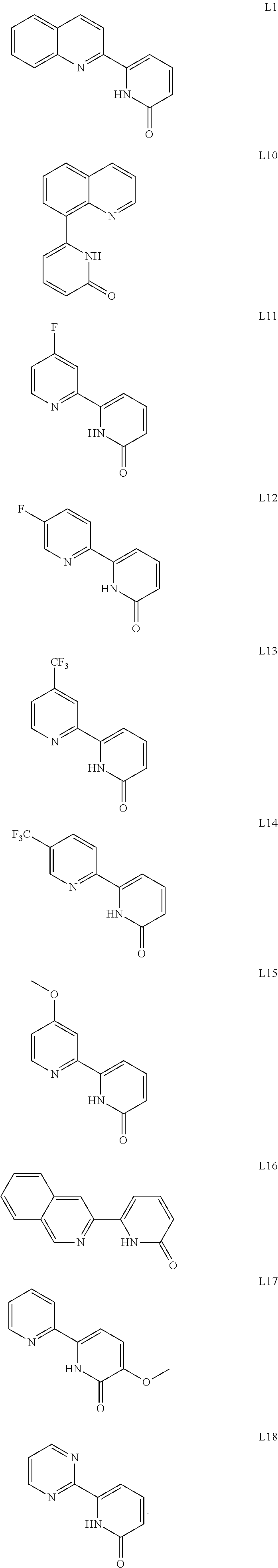

### Embodiment 3

The method of any one of embodiments 1-2, wherein the Pd source is Pd(OAc)₂.

### Embodiment 4

The method of any one of embodiments 1-3, wherein the Ag salt is Ag₂CO₃.

### Embodiment 5

The method of any one of embodiments 1-4, wherein the quinoline-pyridone ligand is L1.

### Embodiment 6

The method of any one of embodiments 1-4, wherein the pyridine-pyridone ligand is L18.

### Embodiment 7

The method of any one of embodiments 1-4, wherein the pyridine-pyridone ligand is L17.

### Embodiment 8

The method of any one of embodiments 1-4, wherein the pyridine-pyridone ligand is L13.

### Embodiment 9

The method of any one of embodiments 1-4, wherein the pyridine-pyridone ligand is L14.

### Embodiment 10

The method of any one of embodiments 1-4, wherein the pyridine-pyridone ligand is L11.

### Embodiment 11

The method of any one of embodiments 1-4, wherein the quinoline-pyridone ligand is L10.

### Embodiment 12

The method of any one of embodiments 1-4, wherein the quinoline-pyridone ligand is L16.

### Embodiment 13

The method of any one of embodiments 1-4, wherein the pyridine-pyridone ligand is L12.

### Embodiment 14

The method of any one of embodiments 1-4, wherein the pyridine-pyridone ligand is L15.

### Embodiment 15

The method of γ-C—H lactonization via β-C—H activation of embodiment 1, wherein the dicarboxylic acid substrate is 1.0 eq. adipic acid, the quinoline-pyridone ligand is 12 mol % L1, the Pd source is 10 mol % Pd(OAc)₂, the Ag salt is 2.0 eq. Ag₂CO₃, with 2.0 eq. BQ3, 1.0 eq. K₂HPO₄ in HFIP at 100° C. for 36 h and the reaction vessel is an 8-10 mL vial.

### Embodiment 16

The method of δ-lactonization via β-C—H activation of embodiment 1, wherein the dicarboxylic acid substrate is 1.0 eq. pimelic acid, the quinoline-pyridone ligand is 12 mol % L1, the Pd source is 10 mol % Pd(OAc)₂, the Ag salt is 2.0 eq. Ag₂CO₃, with 2.0 eq. BQ3, 1.0 eq. K₂HPO₄ in HFIP at 100° C. for 36 h and the reaction vessel is an 8-10 mL vial.

### Embodiment 17

The method of either one of embodiments 15 or 16, wherein the 1.0 eq. K₂HPO₄ is replaced with 0.75 eq. K₂HPO₄.

### Embodiment 18

The method of either one of embodiments 15 or 16, wherein the 1.0 eq. K₂HPO₄ is replaced with a mixture of 0.35 eq. K₂HPO₄ and 0.4 eq. CsOAc.

### Embodiment 19

The method of either one of embodiments 15 or 16, wherein the 2.0 eq. Ag₂CO₃ is replaced with 4.0 eq. MnO₂ and the 1.0 eq. K₂HPO₄ is replaced with K₂HPO₄:KH₂PO₄:CsOAc (1.0:1.5:1.0, 0.75 eq. total).

### Embodiment 20

A method of γ-lactonization via γ-C—H activation comprising i) treating a dicarboxylic acid substrate with a quinoline-pyridone or pyridine-pyridone ligand in the presence of a Pd source; and ii) addition of BQ3, Ag₂CO₃ and K₂HPO₄ in a reaction vessel.

### Embodiment 21

The method of embodiment 20, wherein the quinoline-pyridone or pyridine-pyridone ligand is selected from the group consisting of:

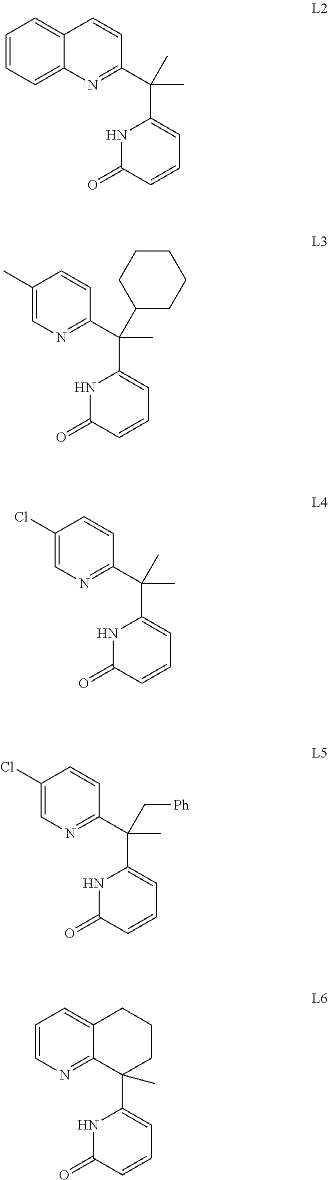

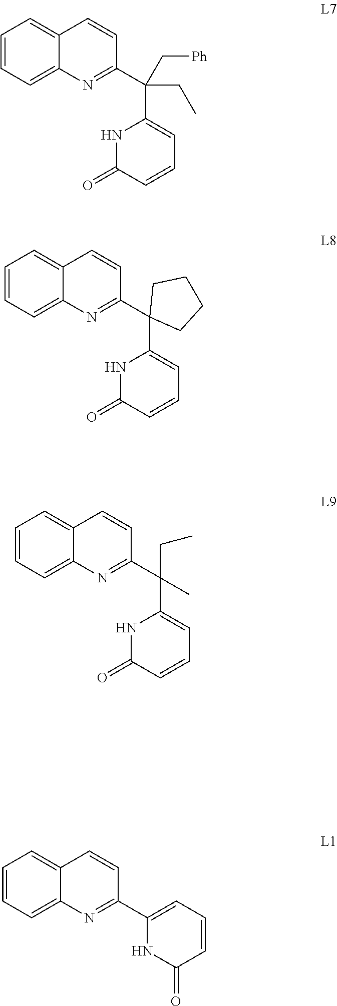 [SMILES: CCC(Cc1ccccc1)(c1ccc2ccccc2n1)c1cccc(=O)[nH]1 ; O=c1cccc(C2(c3ccc4ccccc4n3)CCCC2)[nH]1 ; CCC(C)(c1ccc2ccccc2n1)c1cccc(=O)[nH]1 ; O=c1cccc(-c2ccc3ccccc3n2)[nH]1]

### Embodiment 22

The method of embodiment 21, wherein the quinoline-pyridone ligand is L2.

### Embodiment 23

The method of embodiment 21, wherein the quinoline-pyridone ligand is L9.

### Embodiment 24

The method of embodiment 21, wherein the quinoline-pyridone ligand is L8.

### Embodiment 25

The method of embodiment 21, wherein the quinoline-pyridone ligand is L7.

### Embodiment 26

The method of embodiment 21, wherein the pyridine-pyridone ligand is L5.

### Embodiment 27

The method of embodiment 21, wherein the pyridine-pyridone ligand is L4.

### Embodiment 28

The method of embodiment 21, wherein the quinoline-pyridone ligand is L6.

### Embodiment 29

The method of embodiment 21, wherein the quinoline-pyridone ligand is L1.

### Embodiment 30

A method of γ-lactonization via γ-C—H activation comprising i) treating a dicarboxylic acid substrate with L2 in the presence of a Pd source; and ii) addition of an oxidant and K₂HPO₄.

### Embodiment 31

The method of embodiment 30, wherein the oxidant is 2.0 eq. Na₂S₂O₈.

### Embodiment 32

The method of embodiment 30, wherein the oxidant is 2.0 eq. K₂S₂O₈.

### Embodiment 33

The method of embodiment 30, wherein the oxidant is 2.0 eq. BzOO^{t}Bu.

### Embodiment 34

The method of embodiment 30, wherein the oxidant is 2.0 eq. AcOO^{t}Bu.

### Embodiment 35

The method of embodiment 30, wherein the oxidant is 2.0 eq. Ce(SO₄)₂.

### Embodiment 36

The method of embodiment 30, wherein the oxidant is 2.0 eq. CMHP.

### Embodiment 37

The method of embodiment 30, wherein the oxidant is 2.0 eq. ^{t}BuOO^{t}u.

### Embodiment 38

The method of embodiment 30, wherein the oxidant is 2.0 eq. 1-iodo-3,5-bis(trifluoromethyl)benzene.

### Embodiment 39

The method of embodiment 30, wherein the oxidant is 2.0 eq. methyl 4-iodobenzoate.

### Embodiment 40

The method of embodiment 30, wherein the oxidant is 2.0 eq. 1,2,3,4,5-pentafluoro-6-iodobenzene.

### Embodiment 41

The method of embodiment 30, wherein the oxidant is 2.0 eq. TBHP in H₂O.

### Embodiment 42

The method of embodiment 30, wherein the oxidant is 2.0 eq. H₂O₂ in H₂O.

### Embodiment 43

The method of embodiment 30, wherein the oxidant is selected from the group consisting of:

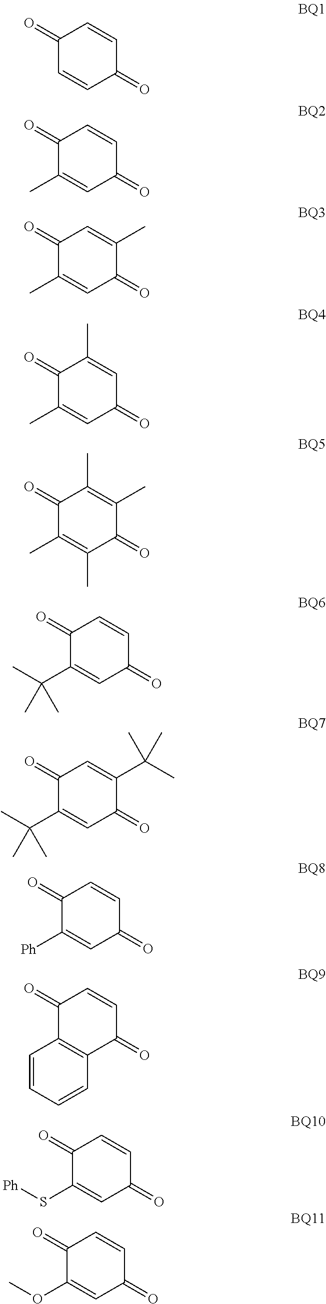

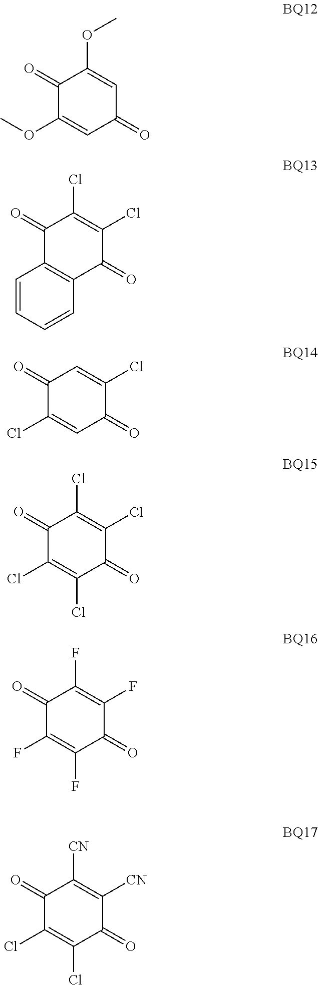

### Embodiment 44

The method of embodiment 43, wherein the oxidant is BQ8.

### Embodiment 45

The method of embodiment 43, wherein the oxidant is BQ5.

### Embodiment 46

The method of embodiment 43, wherein the oxidant is BQ4.

### Embodiment 47

The method of embodiment 43, wherein the oxidant is BQ6.

### Embodiment 48

The method of embodiment 43, wherein the oxidant is BQ1.

### Embodiment 49

The method of embodiment 43, wherein the oxidant is BQ12.

### Embodiment 50

The method of embodiment 43, wherein the oxidant is BQ2.

### Embodiment 51

The method of embodiment 43, wherein the oxidant is BQ9.

### Embodiment 52

The method of embodiment 43, wherein the oxidant is BQ7.

### Embodiment 53

The method of embodiment 43, wherein the oxidant is BQ11.

### Embodiment 54

The method of embodiment 43, wherein the oxidant is BQ13.

### Embodiment 55

The method of embodiment 30, wherein the oxidant is AgOAc.

### Embodiment 56

The method of embodiment 30, wherein the oxidant is Ag2O.

### Embodiment 57

The method of embodiment 30, wherein the oxidant is AgF.

### Embodiment 58

The method of embodiment 30, wherein the oxidant is Ag₂CO₃.

### Embodiment 59

The method of embodiment 30, wherein the oxidant is AgNO₃.

### Embodiment 60

The method of embodiment 30, wherein the oxidant is Ag₃PO₄.

### Embodiment 61

The method of embodiment 30, wherein the oxidant is CuSO₄·5H₂O.

### Embodiment 62

The method of embodiment 30, wherein the oxidant is CuF₂.

### Embodiment 63

The method of embodiment 30, wherein the oxidant is CuO.

### Embodiment 64

The method of embodiment 30, wherein the oxidant is Cu₃(PO₄)₂.

### Embodiment 65

The method of embodiment 30, wherein the oxidant is CuBr₂.

### Embodiment 66

The method of embodiment 30, wherein the oxidant is CuCO₃.

### Embodiment 67

The method of embodiment 30, wherein the oxidant is a mixture of BQ3 and an Ag salt.

### Embodiment 68

The method of embodiment 67, wherein the Ag salt is Ag₂CO₃.

### Embodiment 69

The method of embodiment 67, wherein the Ag salt is Ag₃PO₄.

### Embodiment 70

The method of embodiment 67, wherein the Ag salt is AgF.

### Embodiment 71

The method of embodiment 67, wherein the Ag salt is Ag₂O.

### Embodiment 72

The method of embodiment 67, wherein the Ag salt is AgOAc.

### Embodiment 73

The method of any one of embodiments 20-72, wherein the Pd source is Pd(OAc)₂.

### Embodiment 74

The method of any one of embodiments 20-72, wherein the Pd source is Pd(TFA)₂.

### Embodiment 75

The method of any one of embodiments 20-72, wherein the Pd source is Pd(MeCN)₄(BF₄)₂.

### Embodiment 76

The method of any one of embodiments 20-72, wherein the Pd source is Pd(MeCN)₄(OTf)₂.

### Embodiment 77

The method of any one of embodiments 20-72, wherein the Pd source is PdCl₂.

### Embodiment 78

The method of any one of embodiments 20-72, wherein the Pd source is PdCl₂(PhCN)₂.

### Embodiment 79

The method of any one of embodiments 20-72, wherein the Pd source is Pd₂(dba)₃.

### Embodiment 80

The method of any one of embodiments 20-72, wherein the Pd source is PdCl₂(MeCN)₂.

### Embodiment 81

The method of any one of embodiments 20-80, wherein the dicarboxylic acid substrate is pimelic acid

### Embodiment 82

The method of any one of embodiments 20-80, wherein the dicarboxylic acid substrate is 2,2,6,6-tetramethylpimelic acid.

### Embodiment 83

The method of embodiment 20, wherein the dicarboxylic acid substrate is 1.0 eq. pimelic acid, the quinoline-pyridone ligand is 12 mol % L2, the Pd source is 10 mol % Pd(OAc)₂, the oxidant is 2.0 eq. BQ3 and 2.0 eq. of an Ag salt, and 1.0 eq. K₂HPO₄, the reaction vessel is a vial between 8-10 mL, the reaction temperature is between 80-100° C., and the reaction time is between 12-72 h.

### Embodiment 84

The method of embodiment 83, wherein the Ag salt is Ag₂CO₃.

### Embodiment 85

The method of embodiment 83, wherein the Ag salt is Ag₃PO₄.

### Embodiment 86

The method of embodiment 83, wherein the Ag salt is AgF.

### Embodiment 87

The method of embodiment 83, wherein the Ag salt is Ag₂O.

### Embodiment 88

The method of embodiment 83, wherein the Ag salt is AgOAc.

### Embodiment 89

The method of embodiment 84, wherein the Ag₂CO₃ is replaced by K₂S₂O₈.

### Embodiment 90

The method of embodiment 84, wherein the Ag₂CO₃ is replaced with 0.5 eq. Ag₂CO₃ in addition to replacing the 2.0 eq. BQ3 with 0.5 eq. BQ3.

### Embodiment 91

The method of embodiment 84, wherein the 2.0 eq. Ag₂CO₃ is replaced with 1.0 eq. Ag₂CO₃ in addition to replacing the 2.0 eq. BQ3 with 1.0 eq. BQ3.

### Embodiment 92

The method of any one of embodiments 20-91, wherein the reaction temperature is 100° C.

### Embodiment 93

The method of any one of embodiments 20-91, wherein the reaction temperature is 80° C.

### Embodiment 94

The method of any one of embodiments 20-91, wherein the reaction temperature is 120° C.

### Embodiment 95

The method of any one of embodiments 20-94, wherein the reaction time is 12 h.

### Embodiment 96

The method of any one of embodiments 20-94, wherein the reaction time is 24 h.

### Embodiment 97

The method of any one of embodiments 20-94, wherein the reaction time is 36 h.

### Embodiment 98

The method of any one of embodiments 20-94, wherein the reaction time is 72 h.

### Embodiment 99

The method of any one of embodiments 20-98, wherein the reaction vessel is a 10 mL vial.

### Embodiment 100

The method of any one of embodiments 20-98, wherein the reaction vessel is an 8 mL vial, the reaction temperature is 100° C., and the reaction time is 36 h.

### Embodiment 101

The method of embodiment 83, wherein the 1.0 eq. of K₂HPO₄ is replaced with 0.75 eq. K₂HPO₄ and the reaction vessel is an 8 mL vial, the reaction temperature is 100° C., and the reaction time is 36 h.

### Embodiment 102

The method of embodiment 83, wherein the 1.0 eq. of K₂HPO₄ is replaced with 0.35 eq K₂1HPO₄ and 0.4 eq. CsOAc, the reaction vessel is an 8 mL vial, the reaction temperature is 100° C., and the reaction time is 36 h.

### Embodiment 103

The method of embodiment 83, wherein the 1.0 eq. of K₂HPO₄ is replaced with 0.35 eq K₂HPO₄ and 0.4 eq. CsOAc, L2 is replaced with L10, and the reaction vessel is an 8 mL vial, the reaction temperature is 100° C., and the reaction time is 36 h.

### Embodiment 104

The method of embodiment 83, wherein the 1.0 eq. of K₂HPO₄ is replaced with 0.35 eq K₂HPO₄ and 0.4 eq. CsOAc, L2 is replaced with L10, the reaction vessel is an 8 mL vial, the reaction temperature is 100° C., and the reaction time is 48 h.

### Embodiment 105

The method of embodiment 83, wherein the 2.0 eq. of an Ag salt is replaced with 2.0 eq. of MnO₂.

### Embodiment 106

The method of embodiment 83, wherein the 2.0 eq. of an Ag salt is replaced with 6.0 eq. of MnO₂.

### Embodiment 107

The method of embodiment 83, wherein the 2.0 eq. of an Ag salt is replaced with K₂HPO₄:KH₂PO₄:CsOAc (1:1.5:1, total 0.75 eq.).

### Embodiment 108

The method of embodiment 83, wherein the 2.0 eq. of an Ag salt is replaced with K₂HPO₄:KH₂PO₄:CsOAc (1:1.5:1, total 1.0 eq.).

### Embodiment 109

The method of embodiment 83, wherein the 2.0 eq. of an Ag salt is replaced with K₂HPO₄:KH₂PO₄:CsOAc (1:2:1, total 0.75 eq.).

### Embodiment 110

The method of embodiment 83, wherein the 2.0 eq. of an Ag salt is replaced with K₂HPO₄:KH₂PO₄:CsOAc (1:2:1, total 1.0 eq.).

### Embodiment 111

The method of embodiment 83, wherein the reaction time is 48 h.

### Embodiment 112

The method of embodiment 83, wherein the 2.0 eq. of an Ag salt is replaced with K₂HPO₄:KH₂PO₄:CsOAc (1:1.5:1, total 0.75 eq.) and L2 is replaced with L10.

### Embodiment 113

The method of embodiment 83, wherein the 2.0 eq. of an Ag salt is replaced with K₂HPO₄:KH₂PO₄:CsOAc (1:1.5:1, total 0.75 eq.), L2 is replaced with L10, and the reaction time is 48 h.

### Embodiment 114

The method of embodiment 83, wherein, the oxidant is 2.0 eq. BQ3 and 2.0 eq. of Ag₂CO₃, the reaction vessel is a 10 mL vial, the reaction temperature is 100° C., and the reaction time is 36 h.

### Embodiment 115

The method of embodiment 83, comprising i) treating 1.0 eq. pimelic acid with 12 mol % ligand L2 in the presence of 10 mol % Pd(OAc)₂; and ii) addition of 2.0 eq. Ag₂CO₃, 2.0 eq. BQ3, and 1.0 eq. K₂HPO₄ at 100° C. in HFIP for 36 h in an 8 mL vial.

### Embodiment 116

A method for the total synthesis of Myrotheciumone A. comprising the following steps:
(1) reaction of MeI (2.0 eq.) and K₂CO₃ (3.0 eq.), in acetone, and refluxed for 3 hr;
(2) reaction with Ph₃PCH₃Br (2.9 eq.) and tBuOK (2.4 eq.), in toluene, at r.t. overnight;
(3) reaction with mCPBA (1.2 eq.), in CH₂Cl₂, at r.t. overnight;
(4) reaction with TMSOTf (2.0 eq.) and 2,6-lutidine (2.0 eq.), in toluene, at −78° C. to r.t. overnight;
(5) reaction with cat. Pivalic acid (10 mol %) and triethyl orthoacetate, neat, at 155° C., overnight and further reaction with cat. p-TsOH (10 mol %), in toluene, and refluxed overnight;
(6) reaction with cat. PtO₂, under H₂ (4-layered balloon), in AcOH, at r.t. overnight; (7) reaction with 15% aq. NaOH, and refluxed overnight;
(8) reaction with Pd(OAc)₂ (10 mol %), Ligand L2 (12 mol %), BQ3 (2.0 eq.), Ag₂CO₃ (2.0 eq.), K₂HPO₄ (0.35 eq.), and CsOAc (0.40 eq.), in HFIP, at 100° C., for 36 h; and
(9) reaction with (Ir[dF(CF₃)ppy]₂(dtbpy))PF₆ (1 mol %), Cs₂CO₃ (1.5 eq.), and NaBH₄ (1.2 eq.), under O₂ atmosphere, in CH₂Cl₂, at 40° C., 4×100 W Blue LED lamps, for 40 h.

### Embodiment 117

A method for the total synthesis of Pedicellosine comprising the following steps:
(1) reaction of Pd(OAc)₂ (10 mol %), Ligand L2 (12 mol %), BQ3 (2.0 eq.), Ag₂CO₃ (2.0 eq.), K₂HPO₄ (0.35 eq.), and CsOAc (0.40 eq.), in HFIP, at 100° C., for 36 h;
(2) reaction with BH₃·Me₂S (1.6 eq.), in THF, at 0° C. to r.t. overnight; and
(3) reaction with EDCI (1.5 eq.), DMAP (20 mol %), and 2,3-dihydroxybenzoic acid (1.5 eq.), in CH₂Cl₂, at r.t. overnight.

### Embodiment 118

The application provides any method of γ-lactonization via β-C—H activation, S-lactonization via β—C—H activation, γ-lactonization via γ-C—H activation, or total syntheses of natural products as disclosed herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “ [symbol] ” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds of Formulae I-X may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH— [symbol] —C(—OH)═CH—), amide/imidic acid (—C(═O)—NH— [symbol] —C(—OH)═N—) and amidine (—C(═NR)—NH— [symbol] —C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. [00207]“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R)₂, —CO₂R^{aa}, —SO₂R^{aa}, —SO₂N(R^{cc})2, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆_t₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ¹³H and ¹⁴C) are useful in compound and/or substrate tissue distribution assays. Tritiated (i.e., 3H) and carbon-14 (i.e., ¹³C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

### EXPERIMENTALS

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### GENERAL CONDITIONS

### 1. General Information

Pd(OAc)₂ was purchased from Strem. Solvents were obtained from Sigma-Aldrich, Alfa-Aeser, and Acros, and used directly without further purification. Other reagents were purchased at the highest commercial quality and used without further purification, unless otherwise stated. Analytical thin layer chromatography was performed on 0.25 mm silica gel 60F254 or Merck pre-coated aluminiumbacked silica gel F254 plates. 1H NMR spectra were recorded on Bruker AMX-400, Bruker AV-500, or Bruker DRX-600 instruments. The following abbreviations (or combinations thereof) were used to explain multiplicities: s=singlet, d=doublet, t=triplet, q=quartet, m=multiplet, br=broad. Coupling constants, J, were reported in Hertz unit (Hz). 13C NMR spectra were recorded on Bruker AMX-400, Bruker AV-500, or Bruker DRX-600 and were fully decoupled by broad band proton decoupling. Chemical shifts were referenced to the appropriate residual solvent peaks. Column chromatography was performed using E. Merck silica (60, particle size 0.043-0.063 mm), and pTLC was performed on Merck silica plates (60F254). High-resolution mass spectra (HRMS) were recorded on an Agilent Mass spectrometer using ESI-TOF (electrospray ionization-time of flight).

### 2. Experimental Section for the Lactonization Reaction

### 2.1 Optimization Data for the Lactonization Reactions

### 1. Ligand Investigation for β-Directed, γ-Lactonization:

TABLE S1

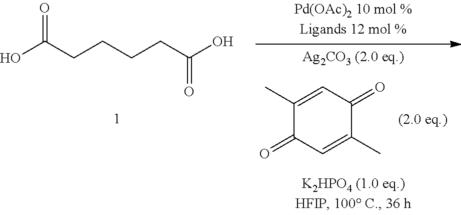

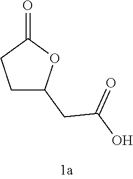

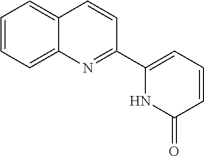 [SMILES: O=c1cccc(-c2ccc3ccccc3n2)[nH]1]

L1 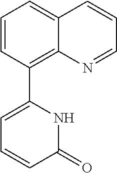 [SMILES: O=c1cccc(-c2cccc3cccnc23)[nH]1]
L10 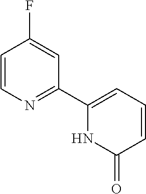 [SMILES: O=c1cccc(-c2cc(F)ccn2)[nH]1]
L11 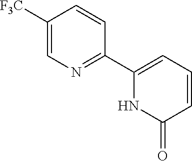 [SMILES: O=c1cccc(-c2ccc(C(F)(F)F)cn2)[nH]1]
L12 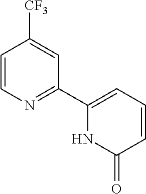 [SMILES: O=c1cccc(-c2cc(C(F)(F)F)ccn2)[nH]1]
L13 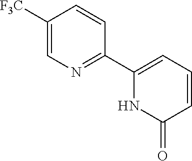 [SMILES: O=c1cccc(-c2ccc(C(F)(F)F)cn2)[nH]1]
L14 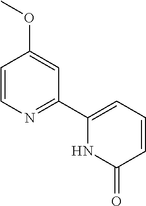
L15 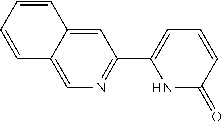 [SMILES: O=c1cccc(-c2cc3ccccc3cn2)[nH]1]
L16 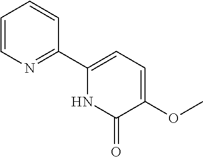
L17 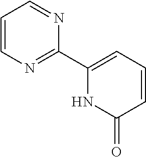 [SMILES: O=c1cccc(-c2ncccn2)[nH]1]
L18
Ligand investigation for β-directed, γ-lactonization.

| | Lactone NMR yield | | Lactone NMR yield |
|---|---|---|---|
| Ligand | (%) | Ligand | (%) |
| L1 | 45 | L16 | 9 |
| L11 | 16 | L17 | 28 |
| L12 | 6 | L18 | 34 |
| L13 | 19 | L1 | 10 |
| L14 | 19 | L10 | 12 |
| L15 | 6 | No L | 3 |

Note: See below for the optimization data leading to the selection of Ag₂CO₃, BQ3, and K₂HPO₄ as the reagents. The optimization process for all the lactonization reactions were carried out simultaneously, with greater focus on the γ-directed, γ-lactonization reaction due to its novelty.

All ligands employed for the exploratory studies are prepared according to literature procedure.(13, 32)

### 2. Fine-Tuning of Reaction Conditions for β-Directed, γ-Lactonization:

TABLE S2

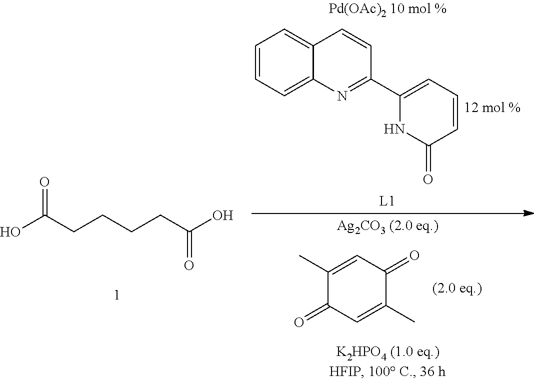

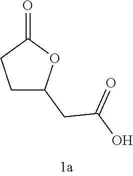

Fine-tuning of reaction conditions for β-directed, γ-lactonization.

| | Lactone NMR yield |
|---|---|
| Deviations from reaction condition as shown | (%) |
| No deviation (Reaction vessel: 10 mL vial) | 45 |
| Reaction vessel: 8 mL vial | 65 |
| 0.75 eq. K₂HPO₄ (Reaction vessel: 8 mL vial) | 65 |
| 0.35. eq K₂HPO₄ + 0.4 eq. CsOAc, (Reaction vessel: 8 mL vial) | 72 |
| Silver free condition: MnO₂ (4.0 eq.) instead of Ag₂CO₃ (2.0 | 46 |
| eq.), K₂HPO₄:KH₂PO₄:CsOAc (1.0:1.5:1.0, 0.75 eq. total) | |

### 3. Ligand Investigation for γ-Directed, γ-Lactonization:

TABLE S3

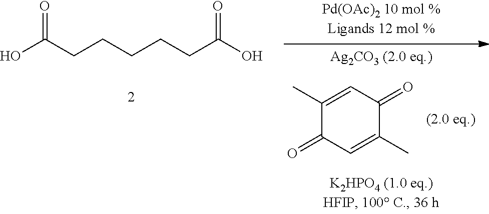

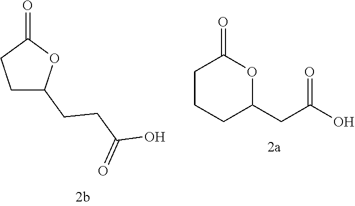 [SMILES: O=C(O)CCC1CCC(=O)O1 ; O=C(O)CC1CCCC(=O)O1]

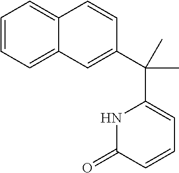 [SMILES: CC(C)(c1ccc2ccccc2c1)c1cccc(=O)[nH]1]

L2 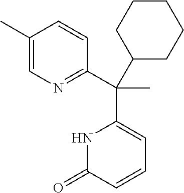 [SMILES: Cc1ccc(C(C)(c2cccc(=O)[nH]2)C2CCCCC2)nc1]
L3 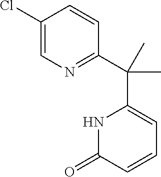 [SMILES: CC(C)(c1ccc(Cl)cn1)c1cccc(=O)[nH]1]
L4 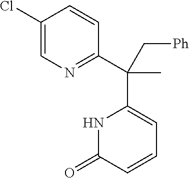 [SMILES: CC(Cc1ccccc1)(c1ccc(Cl)cn1)c1cccc(=O)[nH]1]
L5 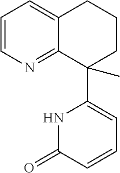 [SMILES: CC1(c2cccc(=O)[nH]2)CCCc2cccnc21]
L6 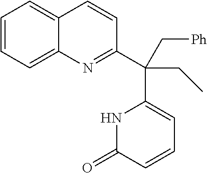 [SMILES: CCC(Cc1ccccc1)(c1ccc2ccccc2n1)c1cccc(=O)[nH]1]
L7 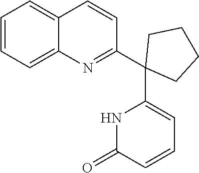 [SMILES: O=c1cccc(C2(c3ccc4ccccc4n3)CCCC2)[nH]1]
L8 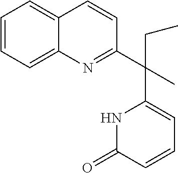 [SMILES: CCC(C)(c1ccc2ccccc2n1)c1cccc(=O)[nH]1]
L9 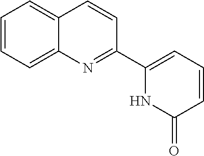 [SMILES: O=c1cccc(-c2ccc3ccccc3n2)[nH]1]
L1
Ligand investigation for γ-directed, γ-lactonization.

| | Lactone NMR yield | | Lactone NMR yield |
|---|---|---|---|
| Ligand | (%) | Ligand | (%) |
| L2 | 52 | L7 | 26 |
| L3 | 0 | L8 | 35 |
| L4 | 20 | L9 | 50 |
| L5 | 21 | L1 | 13 (2a) |
| L6 | 13 | No L | 0 |

### 4. Investigation of Oxidants for γ-Directed, γ-Lactonization:

TABLE S4

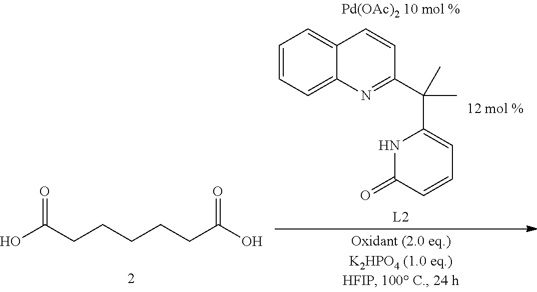 [SMILES: O=C(O)CCCCCC(=O)O ; CC(C)(c1ccc2ccccc2n1)c1cccc(=O)[nH]1]

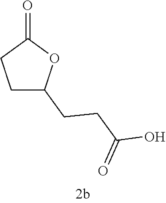 [SMILES: O=C(O)CCC1CCC(=O)O1]

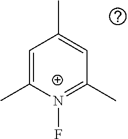

A 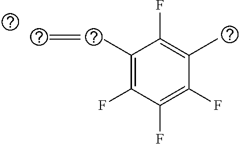
B 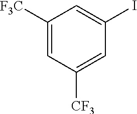 [SMILES: FC(F)(F)c1cc(I)cc(C(F)(F)F)c1]
C 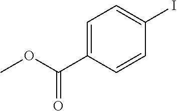
D 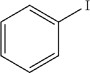
E 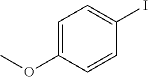
F 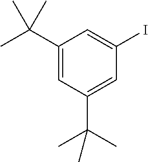
G
Investigation of oxidants for γ-directed, γ-lactonization.

| | NMR yield of 2b | | NMR yield of 2b |
|---|---|---|---|
| Oxidant | (%) | Oxidant | (%) |
| TBHP in H₂O | 2 | BzOO^{t}Bu | 8 |
| CMHP | 5 | BzOOBz | 0 |
| Na₂CO₃•1.5 H₂O₂ | 0 | Lauroyl peroxide | 0 |
| H₂O₂ in H₂O | 2 | ^{t}BuOO^{t}Bu | 5 |
| H₂O₂•urea | 0 | mCPBA | 0 |
| AcOO^{t}Bu | 7 | Na₂S₂O₈ | 12 |
| K₂S₂O₈ | 11 | DMP | 0 |
| (TBA)₂S₂O₈ | 0 | B | 4 |
| Oxone | 0 | C | 5 |
| NFSI | 0 | D | 5 |
| A | 0 | E | 0 |
| Selectfluor | 0 | F | 0 |
| Selectfluor 2 | 0 | G | 0 |
| PIDA | 0 | I2 | 0 |
| PIFA | 0 | Ce(SO₄)₂ | 6 |

### 5. Investigation of Palladium Source for γ-Directed, γ-Lactonization:

TABLE S5

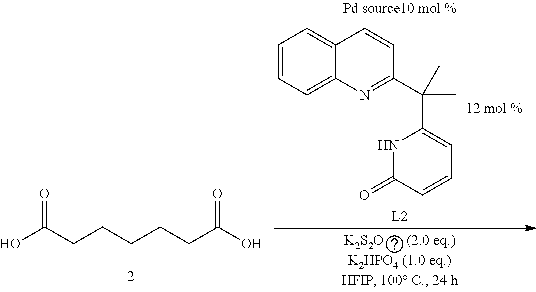 [SMILES: O=C(O)CCCCCC(=O)O ; CC(C)(c1ccc2ccccc2n1)c1cccc(=O)[nH]1]

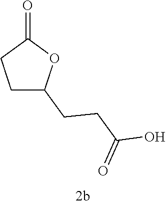 [SMILES: O=C(O)CCC1CCC(=O)O1]

Investigation of palladium source for γ-directed, γ-lactonization.

| Palladium source | NMR yield of 2b (%) |
|---|---|
| Pd(OAc)₂ | 11 |
| PdCl₂(MeCN)₂ | 2 |
| Pd(MeCN)₄(BF₄)₂ | 8 |
| PdCl₂(PhCN)₂ | 3 |
| Pd(TFA)₂ | 13 |
| Pd₂(dba)₃ | 3 |
| PdCl₂ | 4 |
| Pd(MeCN)₄(OTf)₂ | 7 |

### 6. General Control Experiments for γ-Directed, γ-Lactonization:

TABLE S6

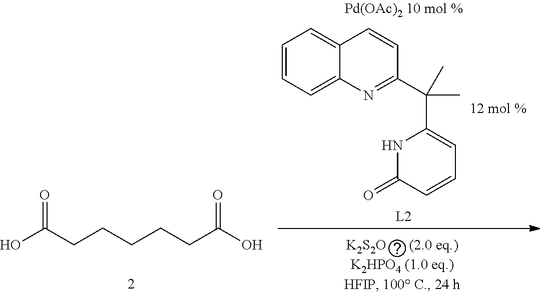 [SMILES: O=C(O)CCCCCC(=O)O ; CC(C)(c1ccc2ccccc2n1)c1cccc(=O)[nH]1]

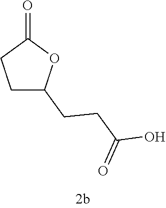 [SMILES: O=C(O)CCC1CCC(=O)O1]

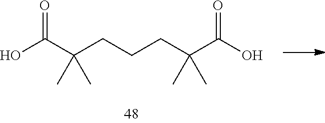

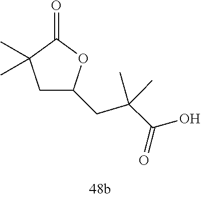

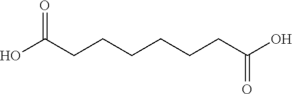 [SMILES: O=C(O)CCCCCCC(=O)O]

H 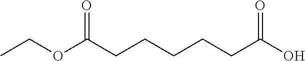
I
General control experiments for γ-directed, γ-lactonization.

| Deviations from reaction condition as | |
|---|---|
| shown | NMR yield of lactone (%) |
| No Palladium | 0 |
| No Ligand | 0 |
| No K₂HPO₄ | 0 |
| 2.0 eq. K₂HPO₄ | 11 |
| No K₂S₂O₈ | 4 |
| 30% Pd and Ligand | 5 |
| With 48 as substrate | 21 (of 48b) |
| With H as substrate | 0 |
| With I as substrate | 0 |

### 7. Investigation of Other Oxidants for γ-Directed, γ-Lactonization:

TABLE S7

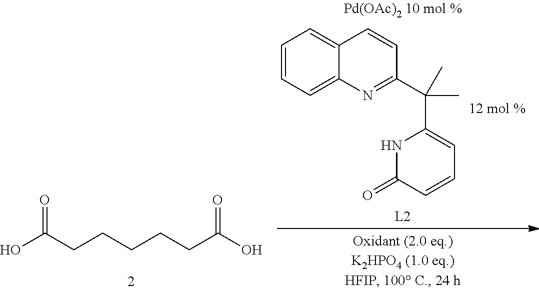 [SMILES: CC(C)(c1ccc2ccccc2n1)c1cccc(=O)[nH]1 ; O=C(O)CCCCCC(=O)O]

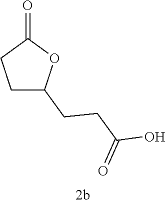 [SMILES: O=C(O)CCC1CCC(=O)O1]

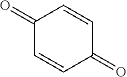

BQ1 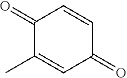
BQ2 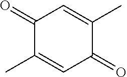
BQ3 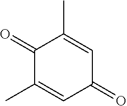
BQ4 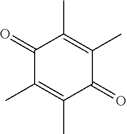
BQ5 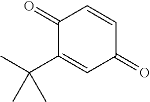
BQ6 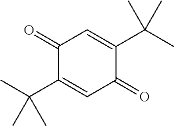 [SMILES: CC(C)(C)C1=CC(=O)C(C(C)(C)C)=CC1=O]
BQ7 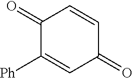 [SMILES: O=C1C=CC(=O)C(c2ccccc2)=C1]
BQ8 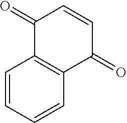 [SMILES: O=C1C=CC(=O)c2ccccc21]
BQ9 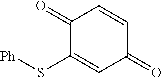 [SMILES: O=C1C=CC(=O)C(Sc2ccccc2)=C1]
BQ10 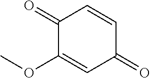
BQ11 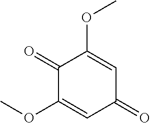
BQ12 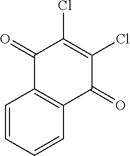 [SMILES: O=C1C(Cl)=C(Cl)C(=O)c2ccccc21]
BQ13 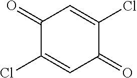
BQ14 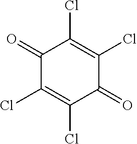 [SMILES: O=C1C(Cl)=C(Cl)C(=O)C(Cl)=C1Cl]
BQ15 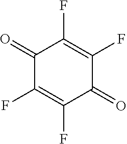 [SMILES: O=C1C(F)=C(F)C(=O)C(F)=C1F]
BQ16 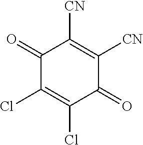 [SMILES: N#CC1=C(C#N)C(=O)C(Cl)=C(Cl)C1=O]
BQ17
Investigation of other oxidants for γ-directed, γ-lactonization.

| | NMR yield of 2b | | NMR yield of 2b |
|---|---|---|---|
| Oxidant | (%) | Oxidant | (%) |
| AgTFA | 0 | BQ1 | 9 |
| AgNO₃ | 7 | BQ2 | 7 |
| Ag₂O | 15 | BQ3 | 19 |
| AgF | 14 | BQ4 | 13 |
| AgOAc | 17 | BQ5 | 16 |
| Ag₃PO₄ | 7 | BQ6 | 10 |
| Ag₂CO₃ | 13 | BQ7 | 4 |
| Cu(OAc)₂ | 0 | BQ8 | 20 |
| CuSO₄•5H₂O | 3 | BQ9 | 5 |
| CuF₂ | 2 | BQ10 | 0 |
| CuO | 2 | BQ11 | 4 |
| CuBr₂ | 1 | BQ12 | 9 |
| CuCl₂ | 0 | BQ13 | 3 |
| Cu(ClO₄)₂ | 0 | BQ14 | 0 |
| Cu₃(PO₄)₂ | 2 | BQ15 | 0 |
| CuCO₃ | 1 | BQ16 | 0 |
| | | BQ17 | 0 |

8. Investigation of Reaction Conditions with Silver Salts and BQ3 for γ-Directed, γ-Lactonization:

TABLE S8

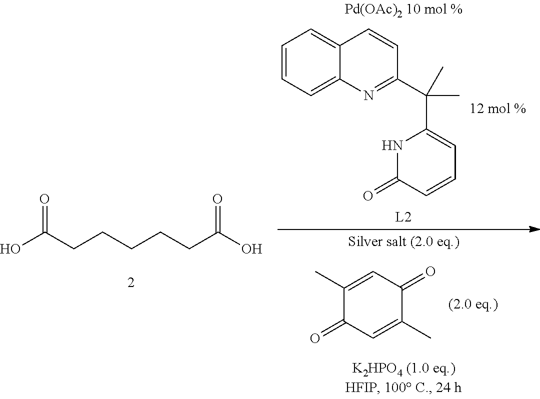

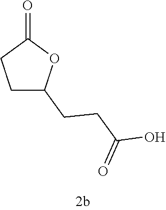 [SMILES: O=C(O)CCC1CCC(=O)O1]

Investigation of reaction conditions with silver salts for γ-directed, γ-lactonization.

| Silver salts | NMR yield of 2b (%) |
|---|---|
| AgOAc | 23 |
| Ag₂O | 27 |
| AgF | 29 |
| Ag₃PO₄ | 41 |
| Ag₂CO₃ | 52 |

TABLE S9

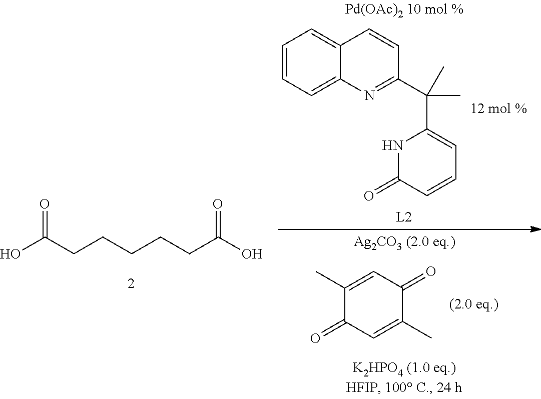

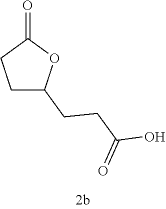 [SMILES: O=C(O)CCC1CCC(=O)O1]

Investigation of reaction conditions with BQ3 for γ-directed, γ-
lactonization.

| Deviations from reaction condition as | |
|---|---|
| shown | NMR yield of 2b (%) |
| Replace Ag₂CO₃ with K₂CO₃ | 0 |
| Replace Ag₂CO₃ with K₂S₂O₈ | 34 |
| 0.5 eq. BQ3 + 0.5 eq. Ag₂CO₃ | 26 |
| 1.0 eq. BQ3 + 1.0 eq. Ag₂CO₃ | 28 |
| Reaction temperature at 80° C. | 40 |
| Reaction temperature at 120° C. | 35 |
| Reaction time: 12 h | 35 |
| Reaction time: 36 h | 52 |
| Reaction time: 72 h | 45 |

### 9. Further Fine-Tuning of Reaction Conditions for γ-Directed, γ-Lactonization:

TABLE S10

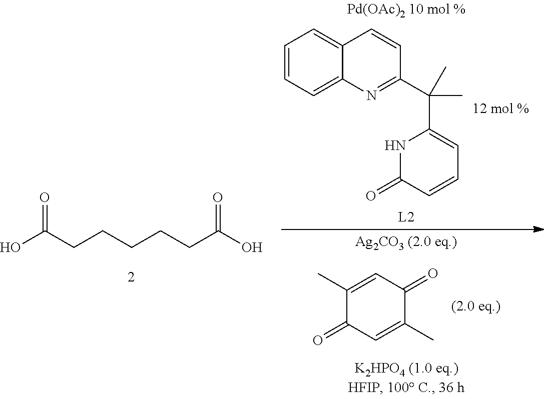

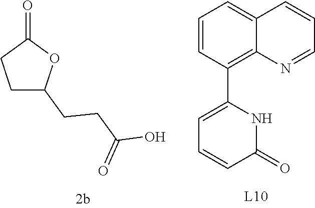 [SMILES: O=C(O)CCC1CCC(=O)O1 ; O=c1cccc(-c2cccc3cccnc23)[nH]1]

Further fine-tuning of reaction conditions for γ-directed, γ-lactonization.

| | Lactone NMR yield |
|---|---|
| Deviations from reaction condition as shown | (%) |
| No deviation (Reaction vessel: 10 mL vial) | 52 |
| Reaction vessel: 8 mL vial | 58 |
| 0.75 eq. K₂HPO₄ (Reaction vessel: 8 mL vial) | 60 |
| 0.35. eq K₂HPO₄ + 0.4 eq. CsOAc, (Reaction vessel: 8 mL vial) | 65 |
| 0.35. eq K₂HPO₄ + 0.4 eq. CsOAc, L10 instead of L2, (Reaction | 69 |
| vessel: 8 mL vial) | |
| 0.35. eq K₂HPO₄ + 0.4 eq. CsOAc, L10 instead of L2, | 75 |
| 48 h reaction time, (Reaction vessel: 8 mL vial) | |

### 10 Optimization for Silver-Free Reaction Conditions Using MnO₂ as Oxidant for γ-Directed, γ-Lactonization:

TABLE S11

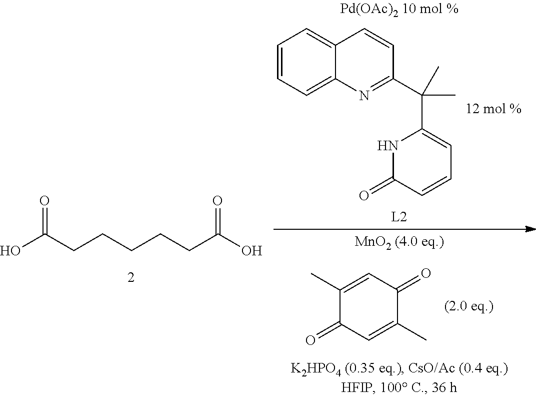

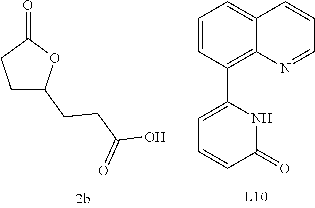 [SMILES: O=C(O)CCC1CCC(=O)O1 ; O=c1cccc(-c2cccc3cccnc23)[nH]1]

Optimization for silver-free reaction conditions using MnO₂ as oxidant
for γ-directed, γ-lactonization.

| Deviations from reaction condition as shown | Lactone NMR yield (%) |
|---|---|
| No deviation (Reaction vessel: 8 mL vial) | 46 |
| 2.0 eq. MnO₂ | 17 |
| 6.0 eq. MnO₂ | 40 |
| K₂HPO₄:KH₂PO₄:CsOAc (1:1.5:1, total 0.75 eq.) | 60 |
| K₂HPO₄:KH₂PO₄:CsOAc (1:1.5:1, total 1.0 eq.) | 34 |
| K₂HPO₄:KH₂PO₄:CsOAc (1:2:1, total 0.75 eq.) | 24 |
| K₂HPO₄:KH₂PO₄:CsOAc (1:2:1, total 1.0 eq.) | 7 |
| Reaction time 48 h | 38 |
| L10 instead of L2, | 62 |
| K₂HPO₄:KH₂PO₄:CsOAc (1:1.5:1, total 0.75 eq.) | |
| L10 instead of L2, | 65 |
| K₂HPO₄:KH₂PO₄:CsOAc (1:1.5:1, total 0.75 eq.), | |
| 48 h reaction time | |

### 2.2 Reaction Procedures of the β-Directed, γ-C—H Lactonization Reaction

General Procedure for the β-Directed, γ-C—H Lactonization Reaction Using Ag₂CO₃ as Oxidant

To a 2-dram vial was added the substrate (0.1 mmol), Pd(OAc)₂ (10 mol %, 0.01 mmol), ligand (12 mol %, 0.012 mmol), p-xyloquinone (0.2 mmol), Ag₂CO₃ (0.2 mmol), K₂HPO₄ (0.035 mmol) and CsOAc (0.04 mmol, preferably added from a stock solution in HFIP as CsOAc is hygroscopic). HFIP (1.0 mL, or the volume needed to make up to 1.0 mL if a stock solution of CsOAc was used) and a stir-bar was then added, followed by sealing the reaction vessel with a PTFE septum inserted between the vial and its cap. (Note: Pd(OAc)₂, ligand, p-xyloquinone, CsOAc, and the substrate could all be prepared as a stock solution in HFIP. The use of stock solution is recommended for setting up a series of reactions to maximize work efficiency). The reaction mixture was sonicated for 30 seconds before stirring at 200 rpm and 100° C. (heating block temperature) for 36 hours. The reaction mixture was then cooled to room temperature and diluted with dichloromethane (1.0 mL), followed by addition of deionized water (2.0 mL), aq. 6M HCl (0.3 mL), brine (1.0 mL) and then shaken vigorously. The lower organic layer was carefully pipetted and filtered through a short plug of Celite®. The remaining aqueous layer was extracted with CH₂Cl₂ (1.0 mL) twice and the organic layer was pipetted and filtered as mentioned. The combined organic layer was then evaporated to dryness. The crude was then taken into CDCl₃ (0.6 mL) with CH₂Br₂ (10.0 μL) as the internal standard to determine the assay yield of the reaction by ¹H NMR spectroscopy. The isolation of the product was carried out with aqueous extractions of the organic layer (in 0.6 mL CDCl₃ diluted with 2.0 mL CH₂Cl₂) with sat. aq. NaHCO₃ solution (1.0 mL each, 3 times). The collected aqueous layer was then acidified by aq. 6M HCl to pH˜2 and extracted with EtOAc (1.0 mL, 3 times). The combined EtOAc layers was dried with anhydrous MgSO₄, filtered, and evaporated to dryness. The product was either further purified by column chromatography (General procedure for gradient elution unless stated otherwise: begin with 20% EtOAc/hexane+1% AcOH, and increase the composition of EtOAc by 10% for every 8 mL of eluent used to 100% EtOAc+1% AcOH, collecting 1.0-2.0 mL fractions. It was observed that the R_{f} values of carboxylic acids obtained by TLC analysis could not be directly used as a guide for column chromatography. Hence, the gradient elution method was employed for their purification.) or pTLC (exact eluent composition mentioned below for each example) or subject to further derivatization into benzyl esters for isolation if purification of the lactone acid was found to be not straightforward.

### General Procedure for the β-Directed, γ-C—H Lactonization Reaction Using MnO₂ as Oxidant

To a 2-dram vial was added the substrate (0.1 mmol), Pd(OAc)₂ (10 mol %, 0.01 mmol), ligand (12 mol %, 0.012 mmol), p-xyloquinone (0.2 mmol), MnO₂ (0.4 mmol), K₂HPO₄ (0.021 mmol), KH₂PO₄ (0.032 mmol), CsOAc (0.021 mmol, preferably added from a stock solution in HFIP as CsOAc is hygroscopic). HFIP (1.0 mL, or the volume needed to make up to 1.0 mL if a stock solution of CsOAc was used) and a stir-bar was then added, followed by sealing the reaction vessel with a PTFE septum inserted between the vial and its cap. (Note: Pd(OAc)₂, ligand, p-xyloquinone, CsOAc, and the substrate could all be prepared as a stock solution in HFIP. The use of stock solution is recommended for setting up a series of reactions to maximize work efficiency). The reaction mixture was sonicated for 30 seconds before stirring at 200 rpm and 100° C. (heating block temperature) for 36-48 hours. The reaction mixture was then cooled to room temperature and diluted with dichloromethane (1.0 mL), followed by addition of deionized water (2.0 mL), aq. 6M HCl (0.3 mL), brine (1.0 mL) and then shaken vigorously. The lower organic layer was carefully pipetted and filtered through a short plug of Celite®. The remaining aqueous layer was extracted with CH₂Cl₂ (1.0 mL) twice and the organic layer was pipetted and filtered as mentioned. The combined organic layer was then evaporated to dryness. The crude was then taken into CDCl₃ (0.6 mL) with CH₂Br₂ (10.0 μL) as the internal standard to determine the assay yield of the reaction by ¹H NMR spectroscopy. The isolation of the product was carried out with aqueous extractions of the organic layer (in 0.6 mL CDCl₃ diluted with 2.0 mL CH₂Cl₂) with sat. aq. NaHCO₃ solution (1.0 mL each, 3 times). The collected aqueous layer was then acidified by aq. 6M HCl to pH˜2 and extracted with EtOAc (1.0 mL, 3 times). The combined EtOAc layers was dried with anhydrous MgSO₄, filtered, and evaporated to dryness. The product was either further purified by column chromatography (General procedure for gradient elution unless stated otherwise: begin with 20% EtOAc/hexane+1% AcOH, and increase the composition of EtOAc by 10% for every 8 mL of eluent used to 100% EtOAc+1% AcOH, collecting 1.0-2.0 mL fractions. It was observed that the R_{f} values of carboxylic acids obtained by TLC analysis could not be directly used as a guide for column chromatography. Hence, the gradient elution method was employed for their purification.) or pTLC (exact eluent composition mentioned below for each example) or subject to further derivatization into benzyl esters for isolation if purification of the lactone acid was found to be not straightforward.

### General Procedure for Benzyl Ester Formation

To the product obtained after aqueous extraction with sat. NaHCO₃ solution as mentioned above was added dry CH₂Cl₂ (2.0 mL), BnOH (1.2 eq.), DMAP (1.2 eq.) and EDCI (1.2 eq.) sequentially at room temperature. The reaction mixture was stirred at room temperature overnight, and completion of the reaction was confirmed by TLC analysis of the reaction mixture. The reaction mixture was then quenched by the addition of water and extracted with CH₂Cl₂ (3 times), and the desired product was purified by pTLC (exact eluent composition mentioned below for each example).

### Guide for Reaction Set-Up, Work-Up, and Expected Observations (Applicable to Both β-Directed and γ-Directed Lactonization Reactions).

Scenario: Setting up 4 reactions for the conversion of pimelic acid 2 to lactone 2b.
1. Weigh Pd(OAc)₂ (5.0 eq., to be dissolved in 1.0 mL HFIP), pimelic acid 2 (5.0 eq., to be dissolved in 1.0 mL HFIP), Ligand L2 (5.0 eq., to be dissolved in 1.0 mL HFIP. Vial is labelled Li in the picture, but it is ligand L2 that was used), BQ3 (4.0 eq., to be dissolved in 0.8 mL HFIP), and CsOAc (5.0 eq., to be dissolved into 1.0 mL HFIP) into 5 separate vials.
2. Weigh Ag₂CO₃ (2.0 eq. each), and K₂HPO₄ (0.35 eq. each) into 4 separate 2-dram vials, whereby each serves as the reaction vessel.
3. Add the required volume of HFIP into the vials containing Pd(OAc)₂ (1.0 mL), pimelic acid 2 (1.0 mL), ligand L2 (1.0 mL), BQ3 (0.8 mL), and CsOAc (1.0 mL). It is recommended to sonicate the mixture to facilitate dissolution of the solids to obtain homogenous solutions.
4. Use an autopipette or a syringe to add 0.2 mL each of the Pd(OAc)₂, pimelic acid 2, ligand L2, p-xyloquinone, and CsOAc solutions into the 4 reaction vessels.
5. Add one stir bar to each of the reaction vessel.
6. Prepare 4 vial caps with PTFE septum (glossy side of the septum should be in contact with the reaction vessel).
7. Put on the caps. Make sure the caps are tightly screwed to the vial and the reaction vessels should therefore be sealed. Tightening too much is not recommended as it would deform the PTFE septum and lead to solvent leakage.
8. Sonicate the reaction mixture, whereby the reaction mixtures will then acquire a milky appearance. Make sure the caps are still tightly screwed to the vial afterwards.
9. Load the reaction vessels onto the heating block, block temperature 100° C., stirring rate 200 rpm, for 36 hours (Do not let the reactions run for longer than 36 hours, as yields were found to decrease with extended reaction time, please see optimization data above).
10. Colour of the reaction mixture changes from bright yellow before heating, to brown/crimson with ligand L2 after ˜15 minutes of heating at 100° C. (block temperature). With ligand L1, the colour changes from bright yellow to olive green (˜5 minutes), and then to brown/crimson (˜15-30 minutes) (not shown here).
11. After 36 hours, cool the reaction mixture to room temperature, dilute with dichloromethane (1.0 mL), followed by addition of deionized water (2.0 mL), aq. 6M aq. HCl (0.3 mL), brine (1.0 mL) and then shaken vigorously. The lower organic layer was carefully pipetted and filtered through a short plug of Celite®.
12. The combined organic layer was then evaporated to dryness. The crude was then taken into CDCl₃ (0.6 mL) with CH₂Br₂ (10.0 L) as the internal standard to determine the assay yield of the reaction by ¹H NMR spectroscopy.

### Determination of Assay Yield by 1H NMR Spectroscopy of the Crude Reaction Mixture:

For 10.0 μL of CH₂Br₂, the no. of mmol of CH₂Br₂ is [(10.0*2.477)/173.83]=0.142 mmol. Since each mole of CH₂Br₂ contains 2 moles of H, the no. of mmol of H is 0.284 mmol.
Setting the integral of the resonance of the CH₂Br₂ standard to 0.284, the integral of each C—H resonance of any species on the crude ¹H NMR would reflect the no. of mmol of that species.
For the case of the lactone product 2b, the resonance at ˜4.5 ppm corresponds to H2a as shown on the structure of the product.
Setting the integral of the CH₂Br₂ standard to 284 would then reflect the percentage NMR yield of the product, because: [(Observed integral of the resonance of H2a when the integral of the standard is set to 0.284)/0.1]*100=Percentage NMR yield of the product.
   2.3 Characterization Data of Products Obtained from β-Directed, γ-C—H Lactonization Reaction

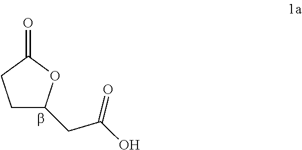

### 2-(5-Oxotetrahydrofuran-2-yl)acetic acid 1a

Following the general procedure for its synthesis, the compound was purified as the acid by column chromatography (General procedure for gradient elution: begin with 50% EtOAc/hexane+1% AcOH, and increase the composition of EtOAc by 10% for every 8 mL of eluent used to 100% EtOAc+1% AcOH). Isolated yield 65% (9.4 mg, 0.065 mmol, colorless oil).

¹H NMR (400 MHz, CDCl₃) δ 4.90 (dddd, J=9.1, 7.8, 6.9, 6.1 Hz, 1H), 2.85 (dd, J=16.6, 6.9 Hz, 1H), 2.72 (dd, J=16.6, 6.1 Hz, 1H), 2.67-2.55 (m, 2H), 2.49 (dt, J=12.8, 7.8 Hz, 1H), 1.99 (dtd, J=12.8, 9.1, 7.7 Hz, 1H).

¹³C NMR (100 MHz, CDCl₃) δ 176.9, 175.0, 76.1, 39.8, 28.6, 27.6.

HRMS (ESI-TOF) Calculated for C₆H₆O₄ [M−H]⁻: 143.0350, found 143.0344.

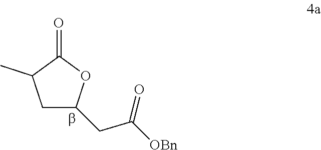 [SMILES: CC1CC(CC(=O)OCc2ccccc2)OC1=O]

### Benzyl 2-(4-methyl-5-oxotetrahydrofuran-2-yl)acetate 4a

Following the general procedure for its synthesis and benzyl ester formation, the compound was purified as the benzyl ester by pTLC (20% EA/hexanes, R_{f}=0.30). Isolated yield 60% over 2 steps (14.9 mg, 0.60 mmol, colourless oil, d.r.=1.5:1). Diastereomers were not separable by column chromatography, ¹H and ¹³C NMR data reported as a mixture of diastereomers.

NMR for minor isomer: ¹H NMR (600 MHz, CDCl₃) δ 7.43-7.32 (m, 5H), 5.15 (s, 2H), 4.97-4.88 (m, 1H), 2.83 (dd, J=16.1, 6.7 Hz, 1H), 2.74-2.59 (m, 2H), 2.26-2.07 (m, 2H), 1.29 (d, J=7.3 Hz, 3H).

¹³C NMR (151 MHz, CDCl₃) δ 179.4, 169.5, 135.5, 128.8, 128.6, 128.5, 74.0, 67.0, 40.0, 35.1, 33.8, 15.9.

NMR for major isomer: ¹H NMR (600 MHz, CDCl₃) δ 7.41-7.30 (m, 5H), 5.15 (s, 2H), 4.82-4.71 (m, 1H), 2.88 (dd, J=16.3, 6.9 Hz, 1H), 2.74-2.59 (m, 2H), 1.65-1.55 (m, 2H), 1.27 (d, J=7.0 Hz, 3H).

¹³C NMR (151 MHz, CDCl₃) δ 178.8, 169.5, 135.5, 128.8, 128.6, 128.5, 74.1, 67.0, 40.2, 37.0, 35.8, 15.1.

HRMS (ESI-TOF) Calculated for C₁₄H17O₄ [M+H]⁺: 249.1127, found 249.1131.

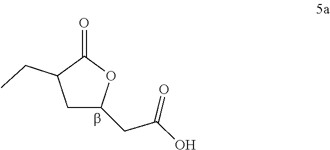

### 2-(4-Ethyl-5-oxotetrahydrofuran-2-yl)acetic acid 5a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 60% (10.3 mg, 0.060 mmol, colorless oil, d.r.=1.4:1). Diastereomers were not separable by column chromatography, ¹H and ¹³C NMR data reported as a mixture of diastereomers.

NMR for minor isomer: ¹H NMR (600 MHz, CDCl₃) δ 4.98-4.86 (m, 1H), 2.90-2.77 (m, 1H), 2.74-2.64 (m, 1H), 2.64-2.54 (m, 1H), 2.24-2.13 (m, 2H), 1.89-1.82 (m, 1H), 1.59-1.53 (m, 1H), 1.01 (t, J=7.5 Hz, 3H).

¹³C NMR (151 MHz, CDCl₃) δ 178.9, 175.2, 74.2, 40.6, 39.9, 32.7, 24.0, 11.7.

NMR for major isomer: ¹H NMR (600 MHz, CDCl₃) δ 4.78-4.72 (m, 1H), 2.89-2.78 (m, 1H), 2.74-2.64 (m, 1H), 2.64-2.55 (m, 2H), 1.97-1.89 (m, 1H), 1.68-1.60 (m, 1H), 1.53-1.46 (m, 1H), 0.99 (t, J=7.5 Hz, 3H).

¹³C NMR (151 MHz, CDCl₃) δ 178.3, 175.2, 74.0, 42.2, 40.0, 34.3, 23.3, 11.7. HRMS (ESI-TOF) Calculated for C₈H₁₁O₄, for [M−H]⁻: 171.0657, found 171.0664.

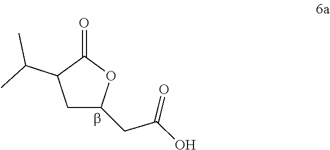

### 2-(4-Isopropyl-5-oxotetrahydrofuran-2-yl)acetic acid 6a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 55% (10.3 mg, 0.055 mmol, colorless oil, d.r. 1.3:1). Diastereomers were not separable by column chromatography, ¹H and ¹C NMR data reported as a mixture of diastereomers.

NMR for minor isomer: ¹H NMR (600 MHz, CDCl₃) δ 4.85 (dt, J=14.0, 6.6 Hz, 1H), 2.82 (dd, J=16.5, 6.7 Hz, 1H), 2.75-2.65 (m, 1H), 2.65-2.57 (m, 1H), 2.34-2.25 (m, 1H), 2.23-2.14 (m, 1H), 2.05 (ddd, J=14.0, 9.9, 5.4 Hz, 1H), 1.09-0.99 (m, 3H), 0.96 (d, J=6.8 Hz, 3H).

¹³C NMR (151 MHz, CDCl₃) δ 177.4, 175.0, 74.2, 45.3, 40.2, 29.3, 29.0, 20.8, 18.7.

NMR for major isomer: ¹H NMR (600 MHz, CDCl₃) δ 4.73 (dq, J=10.5, 6.3 Hz, 1H), 2.87 (dd, J=16.5, 7.0 Hz, 1H), 2.75-2.65 (m, 1H), 2.65-2.57 (m, 1H), 2.43 (ddd, J=13.1, 8.8, 5.8 Hz, 1H), 2.23-2.14 (m, 1H), 1.78-1.67 (m, 1H), 1.09-0.99 (m, 3H), 0.92 (d, J=6.8 Hz, 3H).

¹³C NMR (151 MHz, CDCl₃) δ 178.2, 175.0, 73.7, 46.9, 39.9, 30.3, 27.6, 20.6, 18.4. HRMS (ESI-TOF) Calculated for C₉H₁₅O₄ [M+H]⁺: 187.0970, Found: 187.0972.

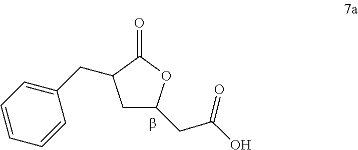 [SMILES: O=C(O)CC1CC(Cc2ccccc2)C(=O)O1]

### 2-(4-Benzyl-5-oxotetrahydrofuran-2-yl)acetic acid 7a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 60% (14.0 mg, 0.060 mmol, colorless oil, d.r=1.4:1). Diastereomers were not separable by column chromatography, ¹H and ¹³C NMR data reported as a mixture of diastereomers.

NMR for minor isomer: ¹H NMR (600 MHz, CDCl₃) δ 7.35-7.15 (m, 5H), 4.79-4.65 (m, 1H), 3.18 (dd, J=13.9, 4.5 Hz, 1H), 3.03-2.92 (m, 1H), 2.83-2.71 (m, 2H), 2.65-2.54 (m, 1H), 2.22 (dt, J=13.1, 7.6 Hz, 1H), 2.03 (ddd, J=13.8, 9.3, 5.1 Hz, 1H).

¹³C NMR (151 MHz, CDCl₃) δ 178.3, 175.3, 138.0, 129.0, 128.9, 127.1, 74.2, 40.9, 39.9, 36.5, 32.2.

NMR for major isomer: ¹H NMR (600 MHz, CDCl₃) δ 7.35-7.15 (m, 5H), 4.79-4.65 (m, 1H), 3.27 (dd, J=14.1, 4.3 Hz, 1H), 3.03-2.92 (m, 1H), 2.83-2.71 (m, 2H), 2.64-2.54 (m, 1H), 2.49-2.40 (m, 1H), 1.71-1.61 (m, 1H).

¹³C NMR (151 MHz, CDCl₃) δ 178.3, 175.3, 138.3, 129.0, 128.9, 126.9, 74.2, 42.6, 39.8, 36.1, 34.3. HRMS (ESI-TOF) Calculated for C₁₃H₁₅O₄ [M+H]⁺: 235.0970, found 235.0967.

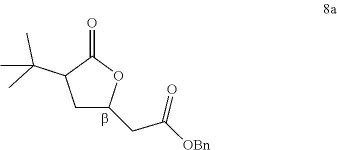 [SMILES: CC(C)(C)C1CC(CC(=O)OCc2ccccc2)OC1=O]

### Benzyl 2-(4-(tert-butyl)-5-oxotetrahydrofuran-2-yl)acetate 8a

Following the general procedure for its synthesis and benzyl ester formation, the compound was purified as the benzyl ester by pTLC (20% EA/hexanes, R_{f}=0.30). Isolated yield 50% over 2 steps (14.5 mg, 0.050 mmol, colourless oil, d.r.=1.5:1). Diastereomers were not separable by column chromatography, ¹H and ¹³C NMR data reported as a mixture of diastereomers.

NMR for minor isomer: ¹H NMR (600 MHz, CDCl₃) δ 7.43-7.28 (m, 5H), 5.30-5.07 (m, 2H), 4.83 (dtd, J=8.1, 6.5, 5.0 Hz, 1H), 2.81 (dd, J=16.1, 6.4 Hz, 1H), 2.64-2.58 (m, 1H), 2.45 (dd, J=9.9, 7.6 Hz, 1H), 2.34 (dt, J=13.5, 7.9 Hz, 1H), 2.03 (ddd, J=13.5, 9.9, 5.0 Hz, 1H), 1.05 (s, 9H).

¹³C NMR (151 MHz, CDCl₃) δ 177.0, 169.6, 135.5, 128.8, 128.6, 128.5, 73.6, 67.0, 48.9, 40.5, 31.7, 30.2, 27.5.

NMR for major isomer: ¹H NMR (600 MHz, CDCl₃) δ 7.43-7.28 (m, 5H), 5.30-5.07 (m, 2H), 4.67 (dtd, J=10.4, 6.5, 5.6 Hz, 1H), 2.88 (dd, J=16.3, 6.7 Hz, 1H), 2.70-2.64 (m, 1H), 2.50 (dd, J=12.6, 8.6 Hz, 1H), 2.43-2.38 (m, 1H), 1.75 (td, J=12.6, 10.5 Hz, 1H), 1.05 (s, 9H).

¹³C NMR (151 MHz, CDCl₃) δ 176.1, 169.6, 135.5, 128.8, 128.7, 128.5, 73.0, 67.0, 50.7, 40.2, 33.0, 31.7, 27.3.

HRMS (ESI-TOF) Calculated for C₁₇H₂₃O₄ [M+H]⁺: 291.1596, Found: 291.1600.

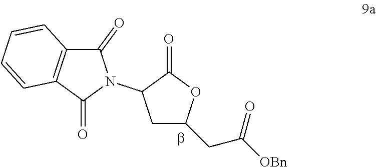 [SMILES: O=C(CC1CC(N2C(=O)c3ccccc3C2=O)C(=O)O1)OCc1ccccc1]

### Benzyl 2-(4-(1,3-dioxoisoindolin-2-yl)-5-oxotetrahydrofuran-2-yl)acetate 9a

Following the general procedure for its synthesis and benzyl ester formation, the compound was purified as the benzyl ester by pTLC (30% EA/hexanes, R_{f}=0.30). Isolated yield 25% over 2 steps (9.5 mg, 0.025 mmol, colourless oil, d.r.=2:1). Diastereomers were not separable by column chromatography, ¹H and ¹³C NMR data reported as a mixture of diastereomers.

NMR for minor diastereomer: ¹H NMR (400 MHz, CDCl₃) δ 8.01-7.82 (m, 2H), 7.82-7.72 (m, 2H), 7.46-7.31 (m, 5H), 5.36-5.11 (m, 4H), 2.96-2.85 (m, 2H), 2.85-2.73 (m, 1H), 2.61-2.44 (m, 1H).

¹³C NMR (100 MHz, CDCl₃) δ 172.2, 169.2, 167.0, 135.4, 134.7, 131.8, 128.9, 128.7, 128.6, 124.0, 74.1, 67.2, 46.7, 39.6, 31.3.

NMR for major diastereomer: ¹H NMR (400 MHz, CDCl₃) δ 8.01-7.82 (m, 2H), 7.82-7.72 (m, 2H), 7.46-7.31 (m, 5H), 5.36-5.11 (m, 3H), 4.99 (tdd, J=8.3, 6.9, 3.6 Hz, 1H), 3.12 (ddd, J=16.5, 6.8, 1.7 Hz, 1H), 2.96-2.85 (m, 1H), 2.85-2.73 (m, 1H), 2.61-2.44 (m, 1H).

¹³C NMR (100 MHz, CDCl₃) δ 171.2, 169.2, 167.0, 135.4, 134.7, 131.7, 128.8, 128.7, 128.6, 124.0, 73.7, 67.1, 48.2, 40.3, 32.4.

HRMS (ESI-TOF) Calculated for C₂₁H₁₈NO₆ [M+H]⁺: 380.1134, Found: 380.1133.

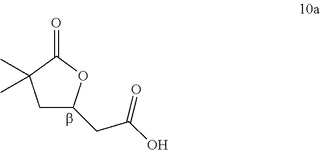

### 2-(4,4-Dimethyl-5-oxotetrahydrofuran-2-yl)acetic acid 10a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 84% (14.4 mg, 0.084 mmol, off white solid).

¹H NMR (500 MHz, CDCl₃) δ 4.83 (ddt, J=10.0, 7.1, 6.0 Hz, 1H), 2.86 (dd, J=16.6, 7.1 Hz, 1H), 2.68 (dd, J=16.6, 6.0 Hz, 1H), 2.32 (dd, J=12.8, 6.0 Hz, 1H), 1.85 (dd, J=12.8, 9.9 Hz, 1H), 1.29 (s, 3H), 1.29 (s, 3H).

¹³C NMR (126 MHz, CDCl₃) δ 181.4, 175.0, 72.5, 43.1, 40.5, 40.0, 25.1, 24.5. HRMS (ESI-TOF) Calculated for C₈H₁₃O₄ [M+H]⁺: 173.0814, Found: 173.0809.

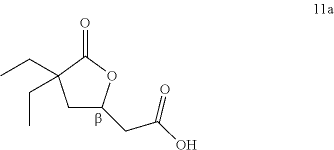

### 2-(4,4-Diethyl-5-oxotetrahydrofuran-2-yl)acetic acid 11a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 66% (13.2 mg, 0.066 mmol, off white solid).

¹H NMR (500 MHz, CDCl₃) δ 4.79 (dddd, J=9.4, 7.0, 6.9, 6.1 Hz, 1H), 2.84 (dd, J=16.5, 7.0 Hz, 1H), 2.66 (dd, J=16.5, 6.1 Hz, 1H), 2.27 (dd, J=13.2, 6.9 Hz, 1H), 1.88 (dd, J=13.2, 9.4 Hz, 1H), 1.75-1.51 (m, 4H), 0.96 (t, J=7.5 Hz, 3H), 0.90 (t, J=7.5 Hz, 3H).

¹³C NMR (126 MHz, CDCl₃) δ 180.4, 175.3, 72.7, 48.8, 40.6, 37.5, 29.3, 28.4, 8.9, 8.8. HRMS (ESI-TOF) Calculated for C₁₀H₁₇O₄ [M+H]⁺: 201.1127, Found: 201.1135.

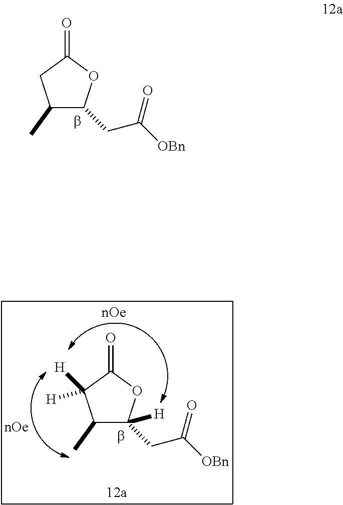

### Benzyl 2-((2R*,3S*)-3-methyl-5-oxotetrahydrofuran-2-yl)acetate 12a

Following the general procedure for its synthesis and benzyl ester formation, the compound was purified as the benzyl ester by pTLC (10% EA/hexanes, R_{f}=0.30). Isolated yield 70% over 2 steps (17.4 mg, 0.070 mmol, colourless oil, d.r.>20:1).

¹H NMR (500 MHz, CDCl₃) δ 7.49-7.29 (m, 5H), 5.16 (s, 2H), 4.49 (td, J=7.0, 5.7 Hz, 1H), 2.77-2.65 (m, 3H), 2.33 (ddd, J=15.3, 8.5, 7.0 Hz, 1H), 2.22 (dd, J=17.3, 9.1 Hz, 1H), 1.15 (d, J=6.6 Hz, 3H).

¹³C NMR (126 MHz, CDCl₃) δ 175.7, 169.7, 135.5, 128.8, 128.6, 128.6, 82.6, 67.1, 39.0, 36.7, 35.9, 17.5.

HRMS (ESI-TOF) Calculated for C₁₄H₁₇O₄ [M+H]⁺: 249.1127, Found: 249.1131.

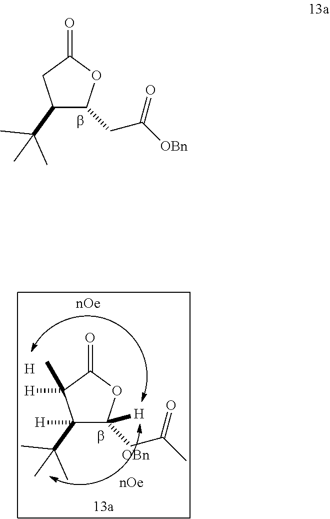

### Benzyl 2-((2R*,3R*)-3-(tert-butyl)-5-oxotetrahydrofuran-2-yl)acetate 13a

Following the general procedure for its synthesis and benzyl ester formation, the compound was purified as the benzyl ester by pTLC (10% EA/hexanes, R_{f}=0.50). Isolated yield 76% over 2 steps (22.0 mg, 0.076 mmol, colourless oil, d.r.>20:1).

¹H NMR (400 MHz, CDCl₃) δ 7.68-7.30 (m, 5H), 5.43-5.01 (m, 2H), 4.79 (ddd, J=6.6, 5.5, 4.4 Hz, 1H), 2.78-2.68 (m, 2H), 2.63 (dd, J=18.4, 10.0 Hz, 1H), 2.40 (dd, J=18.4, 5.4 Hz, 1H), 2.09 (ddd, J=10.0, 5.5, 5.4 Hz, 1H), 0.90 (s, 9H).

¹³C NMR (100 MHz, CDCl₃) δ 176.6, 169.5, 135.4, 128.8, 128.6, 128.6, 77.8, 67.0, 49.5, 41.3, 32.5, 30.4, 26.8.

HRMS (ESI-TOF) Calculated for C₁₇H₂₃O₄ [M+H]⁺: 291.1596, Found: 291.1602.

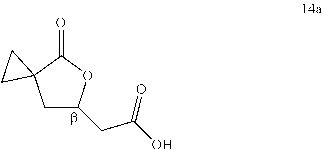 [SMILES: O=C(O)CC1CC2(CC2)C(=O)O1]

### 2-(4-Oxo-5-oxaspiro[2.4]heptan-6-yl)acetic acid 14a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 62% (10.5 mg, 0.062 mmol, off white solid).

¹H NMR (500 MHz, CDCl₃) δ 5.00 (dddd, J=7.6, 7.0, 6.8, 6.5 Hz, 1H), 2.93 (dd, J=16.5, 6.8 Hz, 1H), 2.76 (dd, J=16.5, 6.5 Hz, 1H), 2.48 (dd, J=12.9, 7.6 Hz, 1H), 2.14 (dd, J=12.9, 7.0 Hz, 1H), 1.35 (ddd, J=10.5, 6.5, 3.6 Hz, 1H), 1.26 (ddd, J=10.5, 6.6, 3.7 Hz, 1H), 1.08-0.89 (m, 2H).

¹³C NMR (126 MHz, CDCl₃) δ 179.4, 175.0, 73.4, 40.3, 35.4, 20.3, 16.0, 14.9. HRMS (ESI-TOF) Calculated for C₈H₁₁O₄ [M+H]⁺: 171.0657, Found: 171.0661.

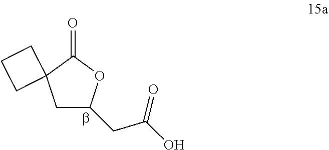 [SMILES: O=C(O)CC1CC2(CCC2)C(=O)O1]

### 2-(5-Oxo-6-oxaspiro[3.4]octan-7-yl)acetic acid 15a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 65% (12.0 mg, 0.065 mmol, off white solid).

¹H NMR (500 MHz, CDCl₃) δ 4.74 (dq, J=8.3, 6.4 Hz, 1H), 2.82 (dd, J=16.5, 7.1 Hz, 1H), 2.68-2.64 (m, 2H), 2.64-2.60 (m, 1H), 2.60-2.53 (m, 1H), 2.46 (dtd, J=11.5, 7.3, 2.6 Hz, 1H), 2.15 (dq, J=11.4, 4.5 Hz, 1H), 2.13-2.08 (m, 1H), 2.08-2.04 (m, 1H), 2.04-2.02 (m, 1H), 2.02-1.98 (m, 2H).

¹³C NMR (126 MHz, CDCl₃) δ 180.5, 175.1, 72.9, 44.3, 41.6, 39.9, 31.8, 29.6, 16.6. HRMS (ESI-TOF) Calculated for C₉H₃O₄ [M+H]⁺: 185.0814, Found: 185.0816.

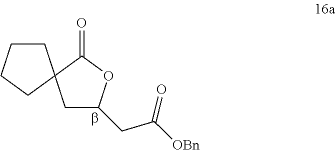 [SMILES: O=C(CC1CC2(CCCC2)C(=O)O1)OCc1ccccc1]

### Benzyl 2-(1-Oxo-2-oxaspiro[4.4]nonan-3-yl)acetate 16a

Following the general procedure for its synthesis and benzyl ester formation, the compound was purified as the benzyl ester by pTLC (20% EA/hexanes, R_{f}=0.30). Isolated yield 55% over 2 steps (15.9 mg, 0.055 mmol, colourless oil).

¹H NMR (600 MHz, CDCl₃) δ 7.50-7.28 (m, 5H), 5.15 (s, 2H), 4.80 (dddd, J=9.4, 6.9, 6.3, 6.0 Hz, 1H), 2.89 (dd, J=16.2, 6.9 Hz, 1H), 2.65 (dd, J=16.2, 6.3 Hz, 1H), 2.33 (dd, J=12.7, 6.0 Hz, 1H), 2.17 (dtd, J=12.9, 6.9, 1.8 Hz, 1H), 1.96-1.87 (m, 1H), 1.87-1.84 (m, 2H), 1.84-1.80 (m, 1H), 1.79-1.71 (m, 1H), 1.71-1.62 (m, 2H), 1.62-1.54 (m, 1H).

¹³C NMR (151 MHz, CDCl₃) δ 181.9, 169.7, 135.5, 128.8, 128.6, 128.5, 73.4, 67.0, 50.1, 42.9, 40.3, 37.6, 36.9, 25.6, 25.5.

HRMS (ESI-TOF) Calculated for C₁H₂₁O₄ [M+H]⁺: 289.1440, Found: 289.1446.

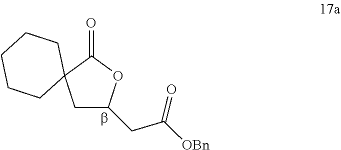 [SMILES: O=C(CC1CC2(CCCCC2)C(=O)O1)OCc1ccccc1]

### Benzyl 2-(1-oxo-2-oxaspiro[4.5]decan-3-yl)acetate 17a

Following the general procedure for its synthesis and benzyl ester formation, the compound was purified as the benzyl ester by pTLC (20% EA/hexanes, R_{f}=0.35). Isolated yield 65% over 2 steps (20.0 mg, 0.065 mmol, colourless oil).

¹H NMR (600 MHz, CDCl₃) δ 7.49-7.29 (m, 5H), 5.16 (s, 2H), 4.82 (dddd, J=10.3, 6.8, 6.4, 6.3 Hz, 1H), 2.88 (dd, J=16.2, 6.8 Hz, 1H), 2.66 (dd, J=16.2, 6.4 Hz, 1H), 2.48 (dd, J=13.0, 6.3 Hz, 1H), 1.88-1.80 (m, 1H), 1.80-1.75 (m, 1H), 1.73 (dd, J=9.7, 4.1 Hz, 1H), 1.71-1.66 (m, 1H), 1.66-1.62 (m, 1H), 1.62-1.58 (m, 2H), 1.48 (dt, J=13.9, 4.5 Hz, 1H), 1.42-1.36 (m, 1H), 1.36-1.30 (m, 1H), 1.28-1.18 (m, 1H).

¹³C NMR (151 MHz, CDCl₃) δ 181.0, 169.7, 135.5, 128.8, 128.6, 128.6, 73.1, 67.0, 45.0, 40.6, 39.3, 34.4, 31.7, 25.4, 22.3, 22.2.

HRMS (ESI-TOF) Calculated for C₁₈H₂₃O₄ [M+H]⁺: 303.1596, Found: 303.1599.

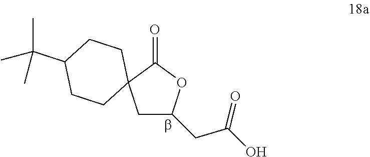 [SMILES: CC(C)(C)C1CCC2(CC1)CC(CC(=O)O)OC2=O]

### 2-(8-(tert-Butyl)-1-oxo-2-oxaspiro[4.5]decan-3-yl)acetic acid 18a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 67% (18.0 mg, 0.067 mmol, off white solid, d.r.=2.5:1). Diastereomers were not separable by column chromatography, ¹H and ¹³C NMR data reported as a mixture of diastereomers.

¹H NMR (500 MHz, CDCl₃) δ 4.91-4.68 (m, 1H), 2.95-2.77 (m, 1H), 2.75-2.61 (m, 1H), 2.52 (dd, J=13.0, 6.5 Hz, 0.4H), 2.20-2.13 (m, 1H), 1.98-1.92 (m, 1H), 1.92-1.87 (m, 1H), 1.87-1.81 (m, 1H), 1.81-1.73 (m, 1H), 1.72-1.65 (m, 0.5H), 1.65-1.57 (m, 2H), 1.57-1.50 (m, 0.4H), 1.46-1.33 (m, 2H), 1.15-1.04 (m, 1H), 0.99-0.92 (m, 1H), 0.89-0.83 (m, 9H).

¹³C NMR (126 MHz, CDCl₃) δ 181.5, 179.1, 175.4, 175.3, 72.9, 72.1, 47.7, 47.2, 45.2, 44.6, 42.4, 40.4, 40.3, 38.4, 35.0, 34.9, 34.9, 32.6, 32.6, 32.0, 27.7, 27.5, 23.4, 23.3, 22.7, 22.7. HRMS (ESI-TOF) Calculated for C₁₅H₂₅O₄ [M+H]⁺: 269.1753, Found: 269.1758.

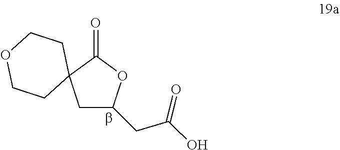 [SMILES: O=C(O)CC1CC2(CCOCC2)C(=O)O1]

### 2-(1-Oxo-2,8-dioxaspiro[4.5]decan-3-yl)acetic acid 19a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 68% (14.5 mg, 0.068 mmol, off white solid).

¹H NMR (500 MHz, CDCl₃) δ 4.85 (dddd, J=9.7, 6.8, 6.2, 6.0 Hz, 1H), 4.07 (dt, J=11.7, 4.7 Hz, 1H), 3.93 (ddd, J=12.0, 5.4, 4.0 Hz, 1H), 3.63 (ddd, J=12.0, 9.2, 3.0 Hz, 1H), 3.52 (td, J=11.7, 3.1 Hz, 1H), 2.88 (dd, J=16.7, 6.8 Hz, 1H), 2.71 (dd, J=16.6, 6.0 Hz, 1H), 2.58 (dd, J=13.0, 6.2 Hz, 1H), 2.26-2.02 (m, 1H), 1.94 (ddd, J=13.4, 9.2, 4.0 Hz, 1H), 1.85 (dd, J=13.0, 9.7 Hz, 1H), 1.69-1.56 (m, 1H), 1.56-1.47 (m, 1H).

¹³C NMR (126 MHz, CDCl₃) δ 179.3, 174.5, 72.7, 64.1, 63.8, 42.2, 40.0, 39.9, 33.8, 32.1. HRMS (ESI-TOF) Calculated for C₁₀H₁₃O₅ [M−H]⁻: 213.0763, Found: 213.0767.

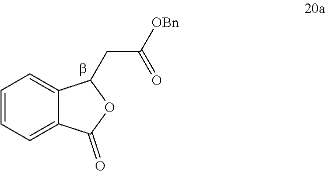 [SMILES: O=C(CC1OC(=O)c2ccccc21)OCc1ccccc1]

### Benzyl 2-β-oxo-1,3-dihydroisobenzofuran-1-yl)acetate 20a

Following the general procedure for its synthesis and benzyl ester formation, the compound was purified as the benzyl ester by pTLC (20% EA/hexanes, R_{f}=0.40). Isolated yield 65% over 2 steps (18.3 mg, 0.065 mmol, colourless oil).

¹H NMR (400 MHz, CDCl₃) δ 7.90 (dt, J=7.6, 1.0 Hz, 1H), 7.64 (td, J=7.6, 1.0 Hz, 1H), 7.54 (tt, J=7.6, 0.8 Hz, 1H), 7.42 (dt, J=7.6, 0.8 Hz, 1H), 7.40-7.31 (m, 5H), 5.90 (dd, J=7.1, 6.2 Hz, 1H), 5.31-5.12 (m, 2H), 3.00 (dd, J=16.5, 7.1 Hz, 1H), 2.91 (dd, J=16.5, 6.2 Hz, 1H).

¹³C NMR (100 MHz, CDCl₃) δ 170.0, 169.2, 148.8, 135.3, 134.4, 129.7, 128.8, 128.7, 128.7, 126.0, 126.0, 122.2, 77.0, 67.2, 39.7.

HRMS (ESI-TOF) Calculated for C₁H₁₅O₄ [M+H]⁺: 283.0970, Found: 283.0973.

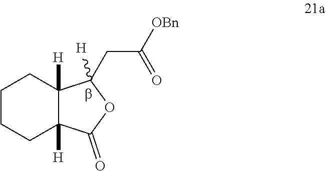 [SMILES: O=C(CC1OC(=O)[C@@H]2CCCC[C@H]12)OCc1ccccc1]

### Benzyl 2-((3aR*,7aS*)-3-oxooctahydroisobenzofuran-1-yl)acetate 21a

Following the general procedure for its synthesis and benzyl ester formation, the compound was purified as the benzyl ester by pTLC (10% EA/hexanes, R_{f}=0.30). Isolated yield 63% over 2 steps (18.2 mg, 0.063 mmol, colourless oil, d.r.=2:1). Diastereomers were not separable by column chromatography, ¹H and ¹³C NMR data reported as a mixture of diastereomers.

¹H NMR (400 MHz, CDCl₃) δ 7.43-7.30 (m, 8H), 4.75 (td, J=7.1, 4.2 Hz, 0.5H), 4.59 (td, J=7.0, 3.0 Hz, 1H), 2.86 (dd, J=16.6, 7.6 Hz, 0.5H), 2.82-2.69 (m, 1.5H), 2.74-2.58 (m, 2.5H), 2.48 (dq, J=11.6, 5.7 Hz, 0.5H), 2.31-2.20 (m, 1H), 1.99 (dt, J=14.2, 4.6 Hz, 1H), 1.89-1.77 (m, 1H), 1.77-1.46 (m, 5.5H), 1.38-1.19 (m, 3H), 1.16-1.05 (m, 1H).

¹³C NMR (100 MHz, CDCl₃) δ 177.8, 177.8, 170.0, 169.8, 135.5, 135.5, 128.8, 128.7, 128.7, 128.7, 128.6, 128.5, 79.0, 76.8, 67.0, 67.0, 41.7, 39.3, 38.6, 38.1, 38.1, 34.5, 27.2, 23.5, 23.1, 23.0, 23.0, 22.9, 22.8, 22.6.

HRMS (ESI-TOF) Calculated for C₁₇H₂₁O₄ [M+H]⁺: 289.1440, Found: 289.1446.

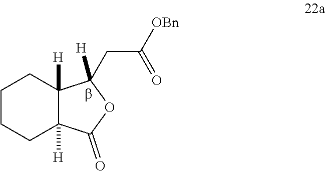 [SMILES: O=C(C[C@@H]1OC(=O)[C@H]2CCCC[C@H]12)OCc1ccccc1]

### Benzyl 2-((1S*,3aS*,7aS*)-3-oxooctahydroisobenzofuran-1-yl)acetate 22a

Following the general procedure for its synthesis and benzyl ester formation, the compound was purified as the benzyl ester by pTLC (10% EA/hexanes, R_{f}=0.30). Isolated yield 60% over 2 steps (17.3 mg, 0.060 mmol, colourless oil, d.r.=5:1). Diastereomers were not separable by column chromatography, ¹H and ¹³C NMR data reported as a mixture of diastereomers.

¹H NMR (500 MHz, CDCl₃) δ 7.48-7.29 (m, 5H), 5.34-5.05 (m, 2H), 4.51 (ddd, J=10.2, 7.5, 4.9 Hz, 1H), 2.76 (dd, J=16.1, 7.5 Hz, 1H), 2.71 (dd, J=16.1, 4.9 Hz, 1H), 2.26-2.09 (m, 1H), 2.02 (ddd, J=13.3, 11.2, 3.3 Hz, 1H), 1.93-1.78 (m, 3H), 1.63 (dtd, J=13.3, 10.2, 2.7 Hz, 1H), 1.31-1.16 (m, 4H).

¹³C NMR (126 MHz, CDCl₃) δ 176.3, 169.8, 135.5, 128.8, 128.6, 128.6, 80.0, 67.1, 48.9, 46.2, 38.4, 27.6, 25.3, 25.0, 25.0.

HRMS (ESI-TOF) Calculated for C₁₇H₂₁O₄ [M+H]⁺: 289.1440, Found: 289.1452.

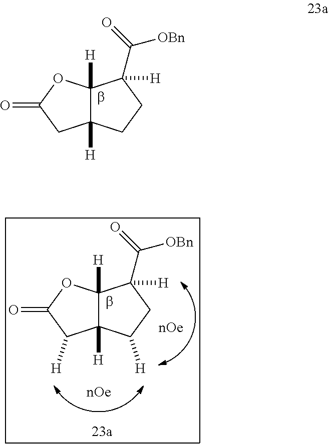

### Benzyl (3aR*,6S*,6aS*)-2-oxohexahydro-2H-cyclopenta[b]furan-6-carboxylate 23a

Following the general procedure for its synthesis and benzyl ester formation, the compound was purified as the benzyl ester by pTLC (30% EA/hexanes, R_{f}=0.50). Isolated yield 20% over 2 steps (5.5 mg, 0.020 mmol, colourless oil).

¹H NMR (500 MHz, CDCl₃) δ 7.41-7.31 (m, 5H), 5.20 (dd, J=6.6, 2.2 Hz, 1H), 5.15 (s, 2H), 3.14 (td, J=7.1, 2.2 Hz, 1H), 2.97 (ddddd, J=9.9, 9.4, 6.6, 6.5, 2.9 Hz, 1H), 2.81 (dd, J=18.4, 9.9 Hz, 1H), 2.33 (dd, J=18.4, 2.9 Hz, 1H), 2.18-2.11 (m, 1H), 2.11-2.03 (m, 1H), 1.96 (dt, J=12.4, 6.3 Hz, 1H), 1.55-1.47 (m, 1H).

¹³C NMR (126 MHz, CDCl₃) δ 176.8, 172.7, 135.7, 128.8, 128.6, 128.3, 87.0, 67.0, 51.1, 38.5, 35.4, 32.6, 28.7.

HRMS (ESI-TOF) Calculated for C₁₅H₁₇O₄ [M+H]⁺: 261.1127, Found: 261.1130.

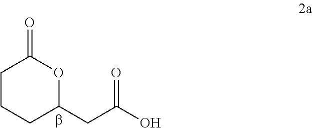 [SMILES: O=C(O)CC1CCCC(=O)O1]

### 2-(6-Oxotetrahydro-2H-pyran-2-yl)acetic acid 2a

Following the general procedure for its synthesis, the compound was purified as the acid by column chromatography. Five parallel reactions were carried out to obtain enough material for effective purification, as purification at 0.1 mmol scale was found difficult. (General procedure for gradient elution: begin with 20% EtOAc/hexane+1% AcOH, and increase the composition of EtOAc by 10% for every 8 mL of eluent used to 100% EtOAc+1% AcOH). Isolated yield from the parallel reactions 25% (20.0 mg, 0.125 mmol, off white solid).

¹H NMR (500 MHz, CDCl₃) δ 4.76 (dddd, J=10.7, 7.3, 5.7, 4.4 Hz, 1H), 2.81 (dd, J=16.4, 7.3 Hz, 1H), 2.75-2.55 (m, 2H), 2.48 (ddd, J=17.8, 9.0, 7.2 Hz, 1H), 2.05 (dq, J=13.7, 4.4 Hz, 1H), 2.00-1.80 (m, 2H), 1.62 (dddd, J=13.7, 10.9, 10.7, 5.8 Hz, 1H).

¹³C NMR (126 MHz, CDCl₃) δ 175.0, 171.6, 76.4, 40.5, 29.4, 27.5, 18.4. HRMS (ESI-TOF) Calculated for C₇H₁₁O₄ [M−H]⁻: 157.0507, Found: 157.0501.

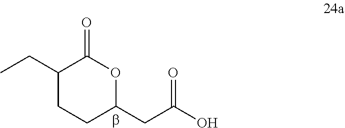

### 2-(5-Ethyl-6-oxotetrahydro-2H-pyran-2-yl)acetic acid 24a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 31% (5.8 mg, 0.031 mmol, colourless oil, d.r.=1:1). Diastereomers were not separable by column chromatography, ¹H and ¹³C NMR data reported as a mixture of diastereomers.

¹H NMR (500 MHz, CDCl₃) δ 4.79-4.66 (m, 1H), 2.93-2.72 (m, 1H), 2.69-2.58 (m, OH), 2.45-2.32 (m, 1H), 2.20-2.13 (m, OH), 2.09-2.01 (m, 1H), 2.00-1.80 (m, 1H), 1.75-1.61 (m, 1H), 1.61-1.53 (m, OH), 1.53-1.44 (m, OH), 1.05-0.94 (m, 2H).

¹³C NMR (126 MHz, CDCl₃) δ 174.9, 174.9, 173.2, 173.2, 77.0, 73.9, 42.1, 40.7, 39.8, 39.6, 28.6, 26.5, 24.9, 24.8, 23.9, 22.8, 11.7, 11.2.

HRMS (ESI-TOF) Calculated for C₉H₁₄O₄Na [M+Na]⁺: 209.0790, Found: 209.0797.

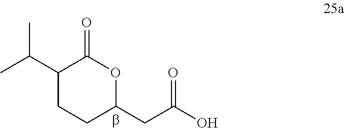

### 2-(5-Isopropyl-6-oxotetrahydro-2H-pyran-2-yl)acetic acid 25a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 30% (6.0 mg, 0.030 mmol, colourless oil, d.r.: 2.2:1). Diastereomers were not separable by column chromatography, ¹H and ¹³C NMR data reported as a mixture of diastereomers.

NMR for minor diastereomer: ¹H NMR (500 MHz, CDCl₃) δ 4.79-4.71 (m, 1H), 2.84-2.74 (m, 1H), 2.68-2.56 (m, 1H), 2.41-2.33 (m, 1H), 2.26 (tt, J=13.2, 6.8 Hz, 1H), 2.06-1.99 (m, 1H), 1.71-1.57 (m, 2H), 1.04-0.90 (m, 6H).

¹³C NMR (126 MHz, CDCl₃) δ 174.4, 174.3, 73.7, 44.0, 39.9, 28.1, 26.9, 20.8, 18.6, 18.4.

NMR for major diastereomer: ¹H NMR (500 MHz, CDCl₃) δ 4.71-4.63 (m, 1H), 2.84-2.74 (m, 1H), 2.68-2.56 (m, 1H), 2.51 (dp, J=11.0, 5.6 Hz, 1H), 2.43 (ddd, J=11.0, 6.9, 3.6 Hz, 1H), 2.12-2.06 (m, 1H), 1.92 (ddd, J=13.6, 7.0, 3.0 Hz, 1H), 1.71-1.57 (m, 4H), 1.04-0.90 (m, 6H).

¹³C NMR (126 MHz, CDCl₃) δ 174.3, 172.9, 73.7, 46.7, 40.8, 29.2, 28.5, 20.0, 20.0, 18.1. HRMS (ESI-TOF) Calculated for C₁₀H₁₇O₄ [M+H]⁺: 201.1127,

Found: 201.1134.

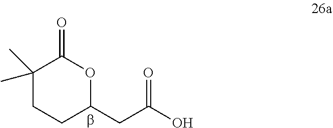

### 2-(5,5-Dimethyl-6-oxotetrahydro-2H-pyran-2-yl)acetic acid 26a

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 31% (5.7 mg, 0.031 mmol, off white solid).

¹H NMR (500 MHz, CDCl₃) δ 4.82-4.68 (m, 1H), 2.79 (dd, J=16.3, 7.1 Hz, 1H), 2.65 (dd, J=16.3, 5.7 Hz, 1H), 2.02-1.90 (m, 1H), 1.87-1.78 (m, 2H), 1.78-1.70 (m, 1H), 1.31 (s, 3H), 1.29 (s, 3H).

¹³C NMR (126 MHz, CDCl₃) δ 177.2, 174.8, 77.4, 40.8, 38.1, 34.4, 27.9, 27.8, 25.8. HRMS (ESI-TOF) Calculated for C₉H₃O₄ [M−H]⁻: 185.0814, Found: 185.0815.

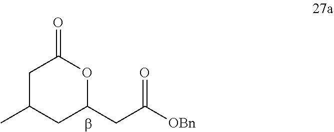 [SMILES: CC1CC(=O)OC(CC(=O)OCc2ccccc2)C1]

### Benzyl 2-(4-methyl-6-oxotetrahydro-2H-pyran-2-yl)acetate 27a

Following the general procedure for its synthesis and benzyl ester formation, the compound was purified as the benzyl ester by pTLC (20% EA/hexanes, R_{f}=0.30). Isolated yield 38% over 2 steps (10.4 mg, 0.040 mmol, colourless oil, d.r.: 1.9:1). Diastereomers were not separable by column chromatography, ¹H and ¹³C NMR data reported as a mixture of diastereomers.

NMR for minor diastereomer: ¹H NMR (600 MHz, CDCl₃) δ 7.44-7.31 (m, 5H), 5.33-5.05 (m, 2H), 4.87 (ddd, J=10.5, 6.7, 4.1 Hz, 1H), 2.93-2.78 (m, 1H), 2.66-2.56 (m, 2H), 2.24-2.15 (m, 2H), 1.83 (ddd, J=14.9, 9.4, 6.2 Hz, 1H), 1.67 (dt, J=14.2, 5.0 Hz, 1H), 1.10 (d, J=6.3 Hz, 3H).

¹³C NMR (151 MHz, CDCl₃) δ 171.7, 169.7, 135.6, 128.8, 128.6, 128.5, 73.4, 67.0, 40.4, 37.4, 34.7, 24.0, 21.4.

NMR for major diastereomer: ¹H NMR (600 MHz, CDCl₃) δ 7.44-7.31 (m, 5H), 5.33-5.05 (m, 2H), 4.74 (dtd, J=12.2, 6.4, 3.0 Hz, 1H), 2.93-2.78 (m, 1H), 2.75-2.66 (m, 1H), 2.66-2.56 (m, 1H), 2.13-2.06 (m, 2H), 2.05-1.97 (m, 1H), 1.27 (dt, J=13.7, 11.5 Hz, 1H), 1.03 (d, J=6.3 Hz, 3H).

¹³C NMR (151 MHz, CDCl₃) δ 170.7, 169.7, 135.6, 128.8, 128.6, 128.5, 76.5, 66.9, 40.9, 38.0, 36.6, 26.7, 21.7.

HRMS (ESI-TOF) Calculated for C₁₅H₁₉O₄ [M+H]⁺: 263.1283, Found: 263.1288.

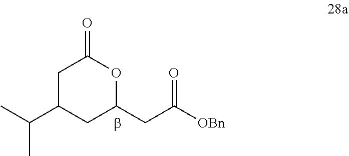 [SMILES: CC(C)C1CC(=O)OC(CC(=O)OCc2ccccc2)C1]

### Benzyl 2-(4-isopropyl-6-oxotetrahydro-2H-pyran-2-yl)acetate 28a

Following the general procedure for its synthesis and benzyl ester formation, the compound was purified as the benzyl ester by pTLC (20% EA/hexanes, R_{f}=0.35). Isolated yield 48% over 2 steps (14.0 mg, 0.048 mmol, colourless oil, d.r.: 3.2:1). Diastereomers were not separable by column chromatography, ¹H and ¹³C NMR data reported as a mixture of diastereomers.

NMR for minor diastereomer: ¹H NMR (500 MHz, CDCl₃) δ 7.45-7.29 (m, 5H), 5.16 (s, 2H), 4.79-4.74 (m, 1H), 2.90-2.79 (m, 1H), 2.70-2.57 (m, 1H), 2.53 (dd, J=15.8, 5.4 Hz, 1H), 2.29 (dd, J=15.8, 10.9 Hz, 1H), 1.83-1.71 (m, 3H), 1.59 (m, 1H), 0.93-0.86 (m, 6H).

¹³C NMR (126 MHz, CDCl₃) δ 172.7, 169.7, 135.6, 128.8, 128.6, 128.5, 73.7, 67.0, 40.2, 35.3, 33.1, 32.3, 30.9, 19.5, 19.3.

NMR for major diastereomer: ¹H NMR (500 MHz, CDCl₃) δ 7.45-7.29 (m, 5H), 5.16 (s, 2H), 4.70 (dtd, J=12.4, 6.4, 2.7 Hz, 1H), 2.90-2.79 (m, 1H), 2.70-2.57 (m, 2H), 2.16 (dd, J=17.8, 10.5 Hz, 1H), 2.00 (ddt, J=13.6, 4.2, 2.2 Hz, 1H), 1.83-1.71 (m, 1H), 1.51 (h, J=6.7 Hz, 1H), 1.28 (dt, J=14.1, 12.2 Hz, 1H), 0.93-0.86 (m, 6H).

¹³C NMR (126 MHz, CDCl₃) δ 171.3, 169.7, 135.6, 128.8, 128.6, 128.5, 76.4, 66.9, 40.9, 37.8, 33.8, 32.4, 32.0, 19.3, 19.2.

HRMS (ESI-TOF) Calculated for C₁₇H₂₂O₄Na [M+Na]⁺: 313.1416, Found: 313.1429.

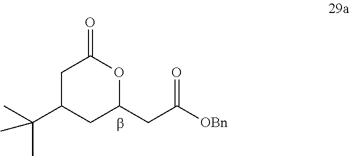 [SMILES: CC(C)(C)C1CC(=O)OC(CC(=O)OCc2ccccc2)C1]

### Benzyl 2-(4-(tert-butyl)-6-oxotetrahydro-2H-pyran-2-yl)acetate 29a

Following the general procedure for its synthesis and benzyl ester formation, the compound was purified as the benzyl ester by pTLC (20% EA/hexanes, R_{f}=0.30). Isolated yield 50% over 2 steps (15.2 mg, 0.050 mmol, colourless oil, d.r.: 5:1). Diastereomers were not separable by column chromatography, ¹H and ¹³C NMR data reported as a mixture of diastereomers.

¹H NMR (500 MHz, CDCl₃) δ 7.53-7.33 (m, 5H), 5.18 (s, 2H), 4.71 (ddt, J=11.6, 6.4, 3.2 Hz, 1H), 2.86 (dd, J=16.0, 6.6 Hz, 1H), 2.67 (dd, J=15.9, 6.1 Hz, 1H), 2.68-2.61 (m, 1H), 2.26 (dd, J=17.8, 10.4 Hz, 1H), 2.00 (ddt, J=13.6, 4.3, 2.3 Hz, 1H), 1.87-1.74 (m, 1H), 1.31 (dt, J=13.3, 11.8 Hz, 1H), 0.89 (s, 9H).

¹³C NMR (126 MHz, CDCl₃) δ 171.6, 169.6, 135.5, 128.7, 128.5, 128.4, 76.3, 66.8, 41.5, 40.8, 32.3, 31.7, 30.1, 26.5.

HRMS (ESI-TOF) Calculated for C₁₈H₂₅O₄ [M+H]⁺: 305.1753, Found: 305.1754.

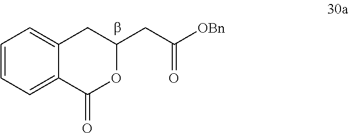 [SMILES: O=C(CC1Cc2ccccc2C(=O)O1)OCc1ccccc1]

### Benzyl 2-(1-oxoisochroman-3-yl)acetate 30a

Following the general procedure for its synthesis and benzyl ester formation, the compound was purified as the benzyl ester by pTLC (20% EA/hexanes, R_{f}=0.30). Isolated yield 71% over 2 steps (21.0 mg, 0.071 mmol, off white solid).

¹H NMR (400 MHz, CDCl₃) δ 8.09 (dd, J=7.8, 1.4 Hz, 1H), 7.55 (td, J=7.5, 1.4 Hz, 1H), 7.45-7.31 (m, 6H), 7.23 (d, J=7.6 Hz, 1H), 5.18 (s, 2H), 5.00 (p, J=6.9 Hz, 1H), 3.08-2.98 (m, 3H), 2.79 (dd, J=16.2, 6.9 Hz, 1H).

¹³C NMR (100 MHz, CDCl₃) δ 169.6, 165.0, 138.5, 135.5, 134.1, 130.5, 128.8, 128.6, 128.5, 128.0, 127.6, 125.0, 74.7, 67.0, 39.9, 32.9.

HRMS (ESI-TOF) Calculated for C₁₈H₁₇O₄ [M+H]⁺: 297.1127, Found: 297.1131.

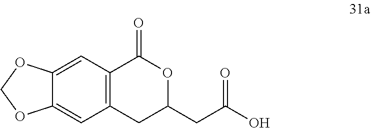 [SMILES: O=C(O)CC1Cc2cc3c(cc2C(=O)O1)OCO3]

### 2-(5-Oxo-7,8-dihydro-5H-[1,3]dioxolo[4,5-g]isochromen-7-yl)acetic acid 31a

Following the general procedure for its synthesis, the compound was purified by pTLC (50% EA/hexanes, 2% AcOH). Isolated yield 75% (18.7 mg, 0.075 mmol, off white solid).

¹H NMR (600 MHz, CD₃OD) δ 7.36 (s, 1H), 6.81 (d, J=0.9 Hz, 1H), 6.08-6.04 (m, 2H), 4.91 (dddd, J=11.3, 7.4, 5.5, 4.0 Hz, 1H), 3.06-2.94 (m, 2H), 2.85-2.74 (m, 2H).

¹³C NMR (151 MHz, CD₃OD) δ 173.4, 166.9, 154.3, 149.0, 137.5, 119.2, 109.6, 108.4, 103.6, 76.7, 40.4, 33.4.

HRMS (ESI-TOF) Calculated for C₁₂H₁₀O₆Na [M+Na]⁺: 273.0375, found 273.0384.

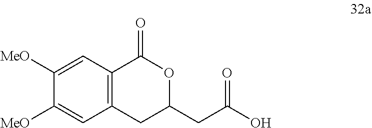 [SMILES: COc1cc2c(cc1OC)C(=O)OC(CC(=O)O)C2]

### 2-(6,7-Dimethoxy-1-oxoisochroman-3-yl)acetic acid 32a

Following the general procedure for its synthesis, the compound was purified by pTLC (50% EA/hexanes, 2% AcOH). Isolated yield 75% (19.9 mg, 0.075 mmol, off white solid).

¹H NMR (600 MHz, CD₃OD) δ 7.49 (s, 1H), 6.91 (s, 1H), 4.94 (dddd, J=11.3, 7.4, 5.6, 4.0 Hz, 1H), 3.92 (s, 3H), 3.86 (s, 3H), 3.09-2.97 (m, 2H), 2.87-2.76 (m, 2H).

¹³C NMR (151 MHz, CD₃OD) δ 173.4, 167.4, 155.9, 150.0, 135.6, 117.6, 112.7, 111.0, 77.0, 56.7, 56.5, 40.5, 33.0.

HRMS (ESI-TOF) Calculated for C₁H₁₅O₆ [M+H]⁺: 267.0869, found 267.0875.

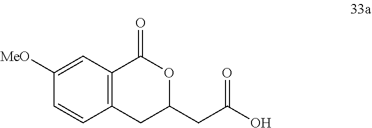 [SMILES: COc1ccc2c(c1)C(=O)OC(CC(=O)O)C2]

### 2-(7-Methoxy-1-oxoisochroman-3-yl)acetic acid 33a

Following the general procedure for its synthesis, the compound was purified by pTLC (50% EA/hexanes, 2% AcOH). Isolated yield 80% (18.9 mg, 0.08 mmol, off white solid).

¹H NMR (600 MHz, CD₃OD) δ 7.49 (d, J=2.7 Hz, 1H), 7.26 (d, J=8.3 Hz, 1H), 7.17 (dd, J=8.4, 2.8 Hz, 1H), 4.93 (dddd, J=11.1, 7.5, 5.6, 3.5 Hz, 1H), 3.83 (s, 3H), 3.07-2.93 (m, 2H), 2.86-2.74 (m, 2H).

¹³C NMR (151 MHz, CD₃OD) δ 172.7, 165.8, 159.2, 131.5, 128.7, 125.2, 121.0, 112.8, 76.0, 54.6, 39.4, 31.2.

HRMS (ESI-TOF) Calculated for C₁₂H₁₃O₅ [M+H]⁺: 237.0763, found 237.0762.

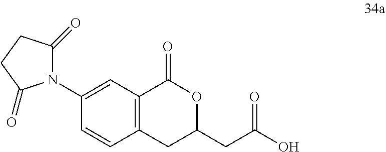 [SMILES: O=C(O)CC1Cc2ccc(N3C(=O)CCC3=O)cc2C(=O)O1]

### 2-(7-(2,5-Dioxopyrrolidin-1-yl)-1-oxoisochroman-3-yl)acetic acid 34a

Following the general procedure for its synthesis, the compound was purified by pTLC (50% EA/hexanes, 2% AcOH). Isolated yield 80% (24.3 mg, 0.08 mmol, off white solid).

¹H NMR (600 MHz, DMSO) δ 7.88-7.84 (m, 1H), 7.55-7.51 (m, 2H), 4.93 (dddd, J=9.4, 7.6, 4.7, 3.2 Hz, 1H), 3.17-3.07 (m, 2H), 2.86-2.72 (m, 6H).

¹³C NMR (151 MHz, DMSO) δ 176.9, 171.1, 163.7, 139.2, 132.4, 132.0, 128.6, 127.8, 124.9, 75.2, 39.3, 31.4, 28.6.

HRMS (ESI-TOF) Calculated for C₁₅H₁₄NO₆ [M+H]⁺: 304.0821, found 304.0820.

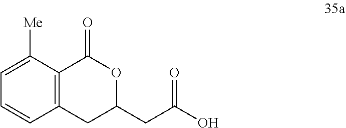 [SMILES: Cc1cccc2c1C(=O)OC(CC(=O)O)C2]

### 2-(8-Methyl-1-oxoisochroman-3-yl)acetic acid 35a

Following the general procedure for its synthesis, the compound was purified by pTLC (50% EA/hexanes, 2% AcOH). Isolated yield 85% (18.7 mg, 0.085 mmol, off white solid).

¹H NMR (600 MHz, CD₃OD) δ 7.44 (t, J=7.6 Hz, 1H), 7.25 (d, J=7.7 Hz, 1H), 7.18 (d, J=7.5 Hz, 1H), 4.84 (dddd, J=11.0, 7.4, 5.6, 3.3 Hz, 1H), 3.12-2.97 (m, 2H), 2.85-2.71 (m, 2H), 2.61 (s, 3H).

¹³C NMR (151 MHz, CD₃OD) δ 173.6, 166.6, 143.8, 141.8, 134.3, 132.1, 126.7, 124.3, 76.2, 40.4, 34.6, 22.2.

HRMS (ESI-TOF) Calculated for C₁₂H₃O₄ [M+H]⁺: 221.0814, found 221.0813.

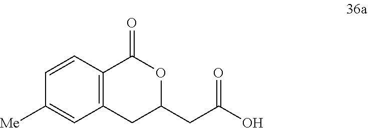 [SMILES: Cc1ccc2c(c1)CC(CC(=O)O)OC2=O]

### 2-(6-Methyl-1-oxoisochroman-3-yl)acetic acid 36a

Following the general procedure for its synthesis, the compound was purified by pTLC (50% EA/hexanes, 2% AcOH). Isolated yield 80% (17.6 mg, 0.08 mmol, off white solid).

¹H NMR (600 MHz, CDCl₃) δ 7.95 (s, 1H), 7.19 (dd, J=8.0, 1.7 Hz, 1H), 7.06 (s, 1H), (p, J=6.9 Hz, 1H), 3.02 (d, J=7.3 Hz, 2H), 2.97 (dd, J=16.5, 6.7 Hz, 1H), 2.79 (dd, J=16.5, 6.2 Hz, 1H), 2.40 (s, 3H).

¹³C NMR (151 MHz, CDCl₃) δ 174.8, 165.4, 145.3, 138.5, 130.6, 129.0, 128.2, 122.1, 74.5, 39.7, 32.8, 21.9.

HRMS (ESI-TOF) Calculated for C₁₂H₃O₄ [M+H]⁺: 221.0814, found 221.0816.

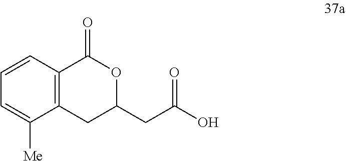 [SMILES: Cc1cccc2c1CC(CC(=O)O)OC2=O]

### 2-(5-Methyl-1-oxoisochroman-3-yl)acetic acid 37a

Following the general procedure for its synthesis, the compound was purified by pTLC (50% EA/hexanes, 2% AcOH). Isolated yield 80% (17.6 mg, 0.080 mmol, off white solid).

¹H NMR (600 MHz, CD₃OD) δ 7.87 (dd, J=7.8, 1.3 Hz, 1H), 7.48 (d, J=7.5 Hz, 1H), 7.31 (t, J=7.7 Hz, 1H), 4.93 (dddd, J=11.5, 6.9, 5.9, 3.3 Hz, 1H), 3.17 (dd, J=16.7, 3.3 Hz, 1H), 2.94-2.87 (m, 1H), 2.85 (dd, J=16.7, 11.5 Hz, 2H), 2.34 (s, 3H).

¹³C NMR (151 MHz, CD₃OD) δ 173.4, 167.6, 139.3, 137.0, 136.6, 128.7, 128.2, 125.7, 76.2, 40.5, 30.5, 18.8.

HRMS (ESI-TOF) Calculated for C₁₂H₁₃O₄ [M+H]⁺: 221.0814, found 221.0824.

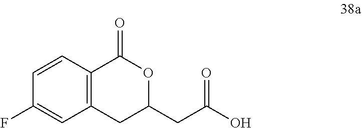 [SMILES: O=C(O)CC1Cc2cc(F)ccc2C(=O)O1]

### 2-(6-Fluoro-1-oxoisochroman-3-yl)acetic acid 38a

Following the general procedure for its synthesis, the compound was purified by pTLC (50% EA/hexanes, 2% AcOH). Isolated yield 68% (15.2 mg, 0.068 mmol, off white solid).

¹H NMR (600 MHz, CD₃OD) δ 8.07 (dd, J=8.6, 5.7 Hz, 1H), 7.20-7.09 (m, 2H), 4.98 (dddd, J=10.0, 7.3, 5.6, 4.2 Hz, 1H), 3.17-3.03 (m, 2H), 2.90-2.75 (m, 2H).

¹³C NMR (151 MHz, CD₃OD) δ 173.4, 167.5 (d, J=254.8 Hz), 166.2, 144.3, 134.1 (d, J=10.1 Hz), 122.4, 116.2 (d, J=22.6 Hz), 115.8 (d, J=22.6 Hz), 76.8, 40.5, 33.1. ¹⁹F NMR (471 MHz, CD₃OD) 5-106.01.

HRMS (ESI-TOF) Calculated for C₁₁H₁₀FO₄ [M+H]⁺: 225.0563, found 225.0560.

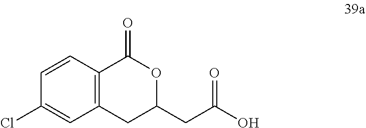 [SMILES: O=C(O)CC1Cc2cc(Cl)ccc2C(=O)O1]

### 2-(6-Chloro-1-oxoisochroman-3-yl)acetic acid 39a

Following the general procedure for its synthesis, the compound was purified by pTLC (50% EA/hexanes, 2% AcOH). Isolated yield 78% (18.7 mg, 0.078 mmol, off white solid).

¹H NMR (600 MHz, CD₃OD) δ 7.98 (d, J=8.2 Hz, 1H), 7.47-7.42 (m, 2H), 4.97 (dddd, J=10.2, 7.3, 5.6, 4.5 Hz, 1H), 3.17-3.05 (m, 2H), 2.88-2.77 (m, 2H).

¹³C NMR (151 MHz, CD₃OD) δ 173.2, 166.2, 142.8, 141.4, 132.6, 129.2, 128.9, 124.6, 76.8, 40.4, 33.1.

HRMS (ESI-TOF) Calculated for C₁₁H₁₀ClO₄ [M+H]⁺: 241.0268, found 241.0264.

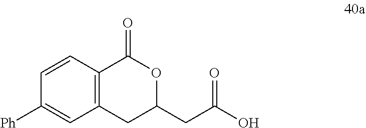 [SMILES: O=C(O)CC1Cc2cc(-c3ccccc3)ccc2C(=O)O1]

### 2-(1-Oxo-6-phenylisochroman-3-yl)acetic acid 40a

Following the general procedure for its synthesis, the compound was purified by pTLC (50% EA/hexanes, 2% AcOH). Isolated yield 75% (21.2 mg, 0.075 mmol, off white solid).

¹H NMR (600 MHz, CD₃OD) δ 8.07 (d, J=8.1 Hz, 1H), 7.71-7.66 (m, 3H), 7.63-7.60 (m, 1H), 7.51-7.45 (m, 2H), 7.44-7.38 (m, 1H), 5.00 (dddd, J=11.0, 7.3, 5.5, 3.6 Hz, 1H), 3.23-3.09 (m, 2H), 2.90-2.80 (m, 2H).

¹³C NMR (151 MHz, CD₃OD) δ 173.4, 167.2, 148.3, 141.4, 140.8, 131.6, 130.1, 129.6, 128.3, 127.4, 127.2, 124.5, 76.9, 40.5, 33.5.

HRMS (ESI-TOF) Calculated for C₁₇H₁₅O₄ [M+H]⁺: 283.0970, found 283.0966.

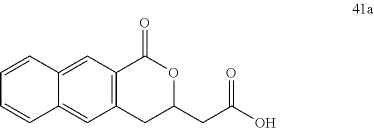 [SMILES: O=C(O)CC1Cc2cc3ccccc3cc2C(=O)O1]

### 2-(1-Oxo-3,4-dihydro-1H-benzo[g]isochromen-3-yl)acetic acid 41a

Following the general procedure for its synthesis, the compound was purified by pTLC (50% EA/hexanes, 2% AcOH). Isolated yield 85% (21.8 mg, 0.085 mmol, off white solid).

¹H NMR (600 MHz, CD₃OD) δ 8.63 (s, 1H), 8.01 (d, J=8.2 Hz, 111), 7.90 (dd, J=8.3, 1.1 Hz, 1H), 7.80 (s, 1H), 7.64 (ddd, J=8.3, 6.8, 1.2 Hz, 1H), 7.55 (ddd, J=8.1, 6.8, 1.2 Hz, 1H), 5.03 (dddd, J=11.0, 7.2, 5.4, 3.3 Hz, 1H), 3.33-3.27 (m, 2H), 3.21 (ddd, J=16.0, 11.0, 1.5 Hz, 1H), 2.93-2.76 (m, 2H).

¹³C NMR (151 MHz, CD₃OD) δ 173.4, 167.5, 137.5, 135.2, 133.5, 133.0, 130.5, 130.3, 128.5, 127.7, 127.2, 123.7, 77.2, 40.6, 33.8.

HRMS (ESI-TOF) Calculated for C₁₅H₁₃O₄ [M+H]⁺: 257.0814, found 257.0819.

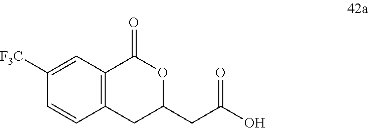 [SMILES: O=C(O)CC1Cc2ccc(C(F)(F)F)cc2C(=O)O1]

### 2-(1-Oxo-7-(trifluoromethyl)isochroman-3-yl)acetic acid 42a

Following the general procedure for its synthesis, the compound was purified by pTLC (50% EA/hexanes, 2% AcOH). Isolated yield 85% (23.3 mg, 0.085 mmol, off white solid).

¹H NMR (600 MHz, CDCl₃) δ 8.37 (s, 1H), 7.81 (dd, J=8.2, 2.0 Hz, 1H), 7.44 (d, J=9.5 Hz, 1H), 5.02 (dddd, J=9.9, 7.0, 6.1, 4.7 Hz, 1H), 3.22-3.12 (m, 2H), 3.02 (dd, J=16.6, 7.0 Hz, 1H), 2.85 (dd, J=16.6, 6.1 Hz, 1H).

¹³C NMR (151 MHz, CDCl₃) δ 174.9, 163.7, 142.1, 131.0 (q, J=33.0 Hz), 130.6 (q, J=3.6 Hz), 128.5, 127.7 (q, J=3.9 Hz), 125.5, 123.5 (q, J=272.3 Hz), 74.4, 39.5, 32.7.

¹⁹F NMR (376 MHz, CDCl₃) δ −65.56.

HRMS (ESI-TOF) Calculated for C₁₂H₁₀O₄F₃ [M+H]⁺: 275.0531, found 275.0529.

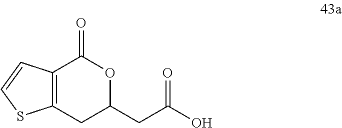 [SMILES: O=C(O)CC1Cc2sccc2C(=O)O1]

### 2-(4-Oxo-6,7-dihydro-4H-thieno[3,2-c]pyran-6-yl)acetic acid 43a

Following the general procedure for its synthesis, the compound was purified by pTLC (50% EA/hexanes, 2% AcOH). Isolated yield 30% (6.4 mg, 0.03 mmol, off white solid).

¹H NMR (600 MHz, CD₃OD) δ 7.40-7.34 (m, 2H), 5.04 (dddd, J=11.3, 7.4, 5.6, 3.7 Hz, 1H), 3.34 (dd, J=16.5, 3.7 Hz, 1H), 3.13 (dd, J=17.0, 11.3 Hz, 1H), 2.85 (qd, J=16.2, 6.5 Hz, 2H).

¹³C NMR (151 MHz, CD₃OD) δ 173.7, 163.4, 151.4, 128.5, 127.1, 125.9, 78.0, 40.6, 29.9. HRMS (ESI-TOF) Calculated for C₉H₉O₄S [M+H]⁺: 213.0222, found 213.0221.

### 2.4 Synthesis and Characterization of Substrates for β-Directed, γ-C—H Lactonization Reaction General Procedure A for the Preparation of α-Substituted Hexanedioic Acids:

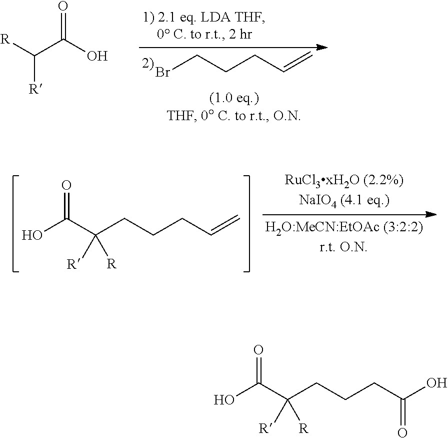

A solution of nBuLi (2.5M in hexanes, 2.1 eq.) was added to a solution of DIPA (2.0 eq.) in THF (0.1M) at 0° C. The resultant solution was stirred at 0° C. for 30 minutes. A carboxylic acid (10.0 mmol, 1.0 eq.) was dissolved in THF (5.0 mL) and added dropwise to the LDA solution at 0° C., in which the solution was warmed to r.t and stirred for 2 hours. The reaction mixture was then cooled to 0° C. and a solution of 5-bromo-1-pentene (10.0 mmol, 1.0 eq.) in THF (5.0 mL) was added. The reaction was warmed to room temperature and stirred overnight. The completion of the reaction was confirmed by TLC analysis of the reaction mixture. The reaction was then quenched with aq. HCl (1.0 M) and extracted three times with EtOAc. The combined organic layer was dried with anhydrous MgSO₄, filtered, and concentrated under vacuum. The crude obtained was directly used for the next step without further purification.

To the crude carboxylic acid obtained in the previous step was added a 3:2:2 ratio of H₂O, MeCN, and EtOAc (0.3 M) at room temperature, followed by RuCl₃·xH₂O (2.2 mol %). The reaction was stirred vigorously and NaIO₄ (4.1 eq.) was added in portions over 1 hour. The reaction mixture was then stirred at room temperature overnight. The reaction mixture was then diluted with water and extracted with EtOAc three times. The combined organic layer was dried with anhydrous MgSO₄, filtered, and concentrated under vacuum. The desired α-substituted hexanedioic acids were obtained after purification by flash column chromatography on silica gel (specific elution conditions are described for each compound below).

### General Procedure B for the Preparation of α-Substituted Hexanedioic Acids:

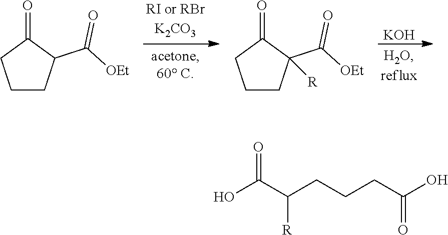

To a stirred suspension of dry potassium carbonate (16.0 mmol) in anhydrous acetone (45 mL) under N₂ was added ethyl 2-oxocyclopentane-1-carboxylate (8.0 mmol) followed by alkyl-iodide or alkyl-bromide (24 mmol). The reaction was heated to 60° C. and stirred overnight. The cooled reaction mixture was diluted with diethyl ether (200 mL) and then filtered. The filtrate was concentrated under reduced pressure and the residue was purified by flash column chromatography on silica gel to give the alkyl-keto ester.

20% aq. KOH (6 mL) was added to a solution 1-alkyl-keto ester (6 mmol) in methanol (6 mL), then the mixture was refluxed and stirred overnight. The cold mixture was extracted with ether. And the aqueous phase solution was acidified with 6M aq. HCl to pH˜2 followed by another extraction with EtOAc (three times). The combined organic layer obtained from extraction of the acidic aqueous layer was dried with anhydrous MgSO₄, filtered, and concentrated under vacuum. The desired product was obtained after recrystallization by Et₂O and hexane.

A solution of nBuLi (2.5M in hexanes, 2.1 eq.) was added to a solution of DIPA (2.0 eq.) in THF (0.1M) at 0° C. The resultant solution was stirred at 0° C. for 30 minutes. A carboxylic acid (10.0 mmol, 1.0 eq.) was dissolved in THE (5.0 mL) and added dropwise to the LDA solution at 0° C., in which the solution was warmed to r.t and stirred for 2 hours. The reaction mixture was then cooled to 0° C. and a solution of 6-bromo-1-hexene (10.0 mmol, 1.0 eq.) in THE (5.0 mL) was added. The reaction was warmed to room temperature and stirred overnight. The completion of the reaction was confirmed by TLC analysis of the reaction mixture. The reaction was then quenched with aq. HCl (1.0 M) and extracted three times with EtOAc. The combined organic layer was dried with anhydrous MgSO₄, filtered, and concentrated under vacuum. The crude obtained was directly used for the next step without further purification.

To the crude carboxylic acid obtained in the previous step was added a 3:2:2 ratio of H₂O, MeCN, and EtOAc (0.3 M) at room temperature, followed by RuCl₃·xH₂O (2.2 mol %). The reaction was stirred vigorously and NaIO₄ (4.1 eq.) was added in portions over 1 hour. The reaction mixture was then stirred at room temperature overnight. The reaction mixture was then diluted with water and extracted with EtOAc three times. The combined organic layer was dried with anhydrous MgSO₄, filtered, and concentrated under vacuum. The desired α-substituted hexanedioic acids were obtained after purification by flash column chromatography on silica gel (specific elution conditions are described for each compound below).

### General Procedure B for the Preparation of α-Substituted Heptanedioic Acids:

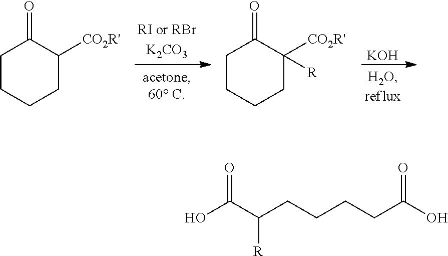

To a stirred suspension of dry potassium carbonate (16.0 mmol) in anhydrous acetone (45 mL) under N₂ was added methyl or ethyl 2-oxocyclohexanecarboxylate (8.0 mmol) followed by alkyl-iodide or alkyl-bromide (24 mmol). The reaction was heated to 60° C. and stirred overnight. The cooled reaction mixture was diluted with diethyl ether and then filtered. The filtrate was concentrated under reduced pressure and the residue was purified by flash column chromatography on silica gel to give the alkyl-keto ester.

20% aq. KOH (6 mL) was added to a solution alkyl-keto ester (6 mmol) in methanol (6 mL), then the mixture was refluxed and stirred overnight. The cold mixture was extracted with ether. And the aqueous phase solution was acidified with 6M aq. HCl to pH˜2 followed by another extraction with EtOAc (three times). The combined organic layer obtained from extraction of the acidic aqueous layer was dried with anhydrous MgSO₄, filtered, and concentrated under vacuum. The desired product was obtained after recrystallization by Et₂O and hexane.

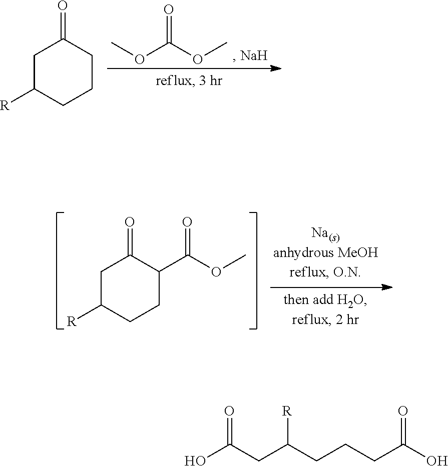

3-Alkylcyclohexanone (8.0 mmol) was dissolved in dimethylcarbonate (15.0 mL) and NaH (0.5 g, 12.2 mmol, 60% dispersion in mineral oil) was added. The reaction mixture was heated to reflux and stirred at this temperature (90° C.) for 3 hours. The reaction was then quenched with sat. NH₄Cl and extracted with EtOAc. The combined organic layer was dried with anhydrous MgSO₄, filtered, and evaporated to dryness to provide the crude β-keto ester.

Sodium metal (1.0 g, 32.0 mmol) was divided into small pieces with a spatula and introduced portionwise to ice-cold anhydrous MeOH (30 mL) with vigorous stirring. After the complete addition of sodium metal, the crude β-keto ester obtained above was dissolved in anhydrous MeOH (5.0 mL) and added dropwise to the ice-cold reaction mixture. The reaction mixture was warmed to room temperature, then heated to reflux, and stirred at this temperature overnight. Water (5.0 mL) was then added to the refluxing reaction mixture and stirring was continued at reflux for 2 hours. The reaction mixture was cooled to room temperature and the MeOH was removed under vacuum. The aqueous residue was extracted with EtOAc three times, and then acidified to pH˜2 with 6M aq. HCl followed by another extraction with EtOAc (three times). The combined organic layer obtained from extraction of the acidic aqueous layer was dried with anhydrous MgSO₄, filtered, and concentrated under vacuum. The desired product was obtained after purification by flash column chromatography on silica gel (specific elution conditions are described for each compound below).

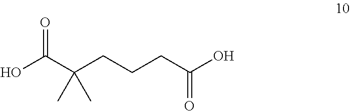

### 2,2-Dimethylhexanedioic acid 10

This compound was prepared according to the general procedure A for the preparation of α-substituted hexanedioic acids.

Elution condition: (20% EA/hexanes+1% AcOH to 50% EA/hexanes+1% AcOH, gradient elution)

Isolated yield: 52% over 2 steps (0.91 g, 5.2 mmol, white solid)

¹H NMR (500 MHz, DMSO) δ 12.03 (s, 2H), 2.32-2.04 (m, 2H), 1.75-1.37 (m, 411), 1.07 (s, 6H).

¹³C NMR (126 MHz, DMSO) δ 178.7, 174.3, 41.1, 39.5, 34.1, 25.0, 24.9, 20.2. HRMS (ESI-TOF) Calculated for C₈H₁₃O₄ [M−H]⁻: 173.0814,

Found: 173.0820.

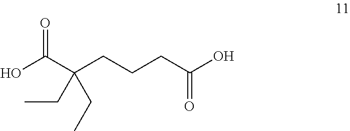

### 2,2-Diethylhexanedioic acid 11

This compound was prepared according to the general procedure A for the preparation of α-substituted hexanedioic acids.

Elution condition: (20% EA/hexanes+1% AcOH to 50% EA/hexanes+1% AcOH, gradient elution)

Isolated yield: 60% over 2 steps (1.21 g, 6.0 mmol, white solid)

¹H NMR (500 MHz, DMSO) δ 12.04 (s, 2H), 2.19 (t, J=7.0 Hz, 2H), 1.55-1.40 (m, 6H), 1.40-1.28 (m, 2H), 0.73 (t, J=7.4 Hz, 6H).

¹³C NMR (126 MHz, DMSO) δ 177.8, 174.3, 48.4, 34.1, 32.3, 26.2, 19.1, 8.2. HRMS (ESI-TOF) Calculated for C₁₀H₁₇O₄ [M−H]⁻: 201.1127,

Found: 201.1128.

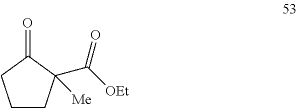 [SMILES: CCOC(=O)C1(C)CCCC1=O]

### Ethyl 1-methyl-2-oxocyclopentane-1-carboxylate 53

This compound was prepared according to the general procedure B for the preparation of α-substituted hexanedioic acids by the reaction with MeI. Quantative yield, (1.36 g, 8.0 mmol, colorless oil).

¹H NMR (600 MHz, CDCl₃) δ 4.20-4.07 (m, 2H), 2.54-2.50 (m, 1H), 2.50-2.39 (m, 1H), 2.35-2.26 (m, 1H), 2.10-2.00 (m, 1H), 1.97-1.81 (m, 2H), 1.30 (s, 3H), 1.24 (td, J=7.1, 0.5 Hz, 3H).

¹³C NMR (151 MHz, CDCl₃) δ 216.1, 172.5, 61.5, 56.0, 37.8, 36.3, 19.7, 19.5, 14.2.

The data is consistent with those reported in the literature.(33)

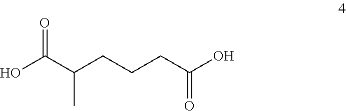

### 2-Methylhexanedioic acid 4

This compound was prepared according to the general procedure B for the preparation of α-substituted hexanedioic acids. 87% yield, (0.84 g, 5.22 mmol, white solid).

¹H NMR (600 MHz, CDCl₃) δ 2.52-2.45 (m, 1H), 2.44-2.33 (m, 2H), 1.79-1.65 (m, 3H), 1.55-1.46 (m, 1H), 1.20 (dd, J=7.0, 1.0 Hz, 3H).

¹³C NMR (151 MHz, CDCl₃) δ 182.5, 179.8, 47.0, 34.1, 31.1, 25.3, 22.7, 11.8. HRMS (ESI-TOF) Calculated for C₇H₁₁O₄ [M−H]⁻: 159.0657, found 159.0653.

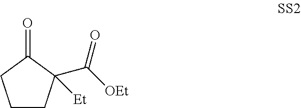 [SMILES: CCOC(=O)C1(CC)CCCC1=O]

### Ethyl 1-ethyl-2-oxocyclopentane-1-carboxylate SS2

This compound was prepared according to the general procedure B for the preparation of α-substituted hexanedioic acids by the reaction with EtI. 95% yield, (1.4 g, 7.6 mmol, colorless oil).

¹H NMR (600 MHz, CDCl₃) δ 4.22-4.10 (m, 2H), 2.55-2.46 (m, 1H), 2.46-2.36 (m, 1H), 2.33-2.19 (m, 1H), 2.05-1.84 (m, 4H), 1.68-1.58 (m, 1H), 1.29-1.21 (m, 3H), 0.89 (t, J=7.5 Hz, 3H).

¹³C NMR (151 MHz, CDCl₃) δ 215.3, 171.3, 77.4, 77.2, 77.0, 61.4, 61.0, 38.2, 38.2, 32.4, 26.9, 19.7, 14.2, 9.3.

The data is consistent with those reported in the literature.(33)

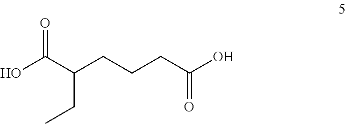

### 2-Ethylhexanedioic acid 5

This compound was prepared according to the general procedure B for the preparation of α-substituted hexanedioic acids. 85% yield, (0.89 g, 5.1 mmol, white solid).

¹H NMR (600 MHz, CDCl₃) δ 2.44-2.29 (m, 3H), 1.86-1.38 (m, 6H), 0.95 (t, J=7.4 Hz, 3H).

¹³C NMR (151 MHz, CDCl₃) δ 182.5, 179.8, 47.0, 34.1, 31.1, 25.3, 22.7, 11.8. HRMS (ESI-TOF) Calculated for C₈H₁₃O₄ [M−H]⁻: 173.0814, found 173.0810.

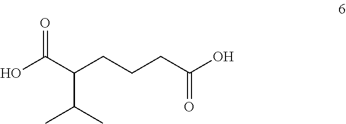

### 2-Isopropylhexanedioic acid 6

This compound was prepared according to the general procedure A for the preparation of α-substituted hexanedioic acids.

Elution condition: (20% EA/hexanes+1% AcOH to 50% EA/hexanes+1% AcOH, gradient elution)

Isolated yield: 42% over 2 steps (791 mg, 4.2 mmol, colourless oil)

¹H NMR (600 MHz, DMSO) δ 12.03 (s, 1H), 2.28-2.12 (m, 1H), 1.97 (td, J=8.2, 4.8 Hz, OH), 1.78-1.69 (m, J=6.8 Hz, 1H), 1.52-1.33 (m, 2H), 0.87 (dd, J=6.8, 4.2 Hz, 3H).

¹³C NMR (151 MHz, DMSO) δ 176.3, 174.3, 51.9, 33.6, 29.9, 28.5, 22.9, 20.4, 19.9. HRMS (ESI-TOF) Calculated for C₉H₁₅O₄ [M−H]⁻: 187.0970, Found: 187.0978.

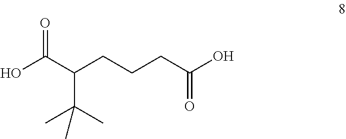

### 2-(tert-Butyl)hexanedioic acid 8

This compound was prepared according to the general procedure A for the preparation of α-substituted hexanedioic acids.

Elution condition: (20% EA/hexanes+1% AcOH to 50% EA/hexanes+1% AcOH, gradient elution)

Isolated yield: 35% over 2 steps (710 mg, 3.5 mmol, colourless oil)

¹H NMR (500 MHz, DMSO) δ 12.00 (s, 2H), 2.36-2.09 (m, 2H), 1.99 (dd, J=11.3, 2.5 Hz, 1H), 1.53-1.39 (m, 3H), 1.36 (ddd, J=13.1, 10.6, 5.0 Hz, 1H), 0.90 (s, 9H).

¹³C NMR (126 MHz, DMSO) δ 176.1, 174.3, 55.5, 33.6, 32.1, 27.5, 26.6, 23.6. HRMS (ESI-TOF) Calculated for C₁₀H₁₇O₄ [M−H]⁻: 201.1127, Found: 201.1124.

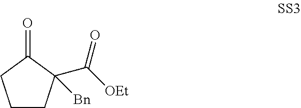 [SMILES: CCOC(=O)C1(Cc2ccccc2)CCCC1=O]

### Ethyl 1-benzyl-2-oxocyclopentane-1-carboxylate SS3

This compound was prepared according to the general procedure B for the preparation of α-substituted hexanedioic acids by the reaction With BnBr. 95% yield, (2.34 g, 7.6 mmol, colorless oil).

¹H NMR (600 MHz, CDCl₃) δ 7.29-7.19 (m, 3H), 7.17-7.12 (m, 2H), 4.22-4.15 (m, 2H), 3.20 (dd, J=13.8, 1.4 Hz, 1H), 3.13 (dd, J=13.9, 1.5 Hz, 1H), 2.45-2.40 (m, 1H), 2.40-2.33 (m, 1H), 2.09-1.84 (m, 3H), 1.65-1.57 (m, 1H), 1.29-1.23 (m, 3H).

¹³C NMR (151 MHz, CDCl₃) δ 215.1, 171.1, 136.8, 130.3, 128.5, 127.0, 77.4, 77.2, 76.9, 61.7, 61.6, 39.1, 38.5, 31.9, 19.6, 14.2.

The data is consistent with those reported in the literature.(34)

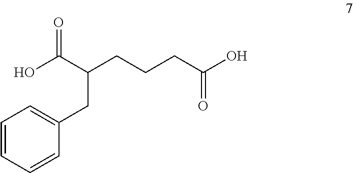 [SMILES: O=C(O)CCCC(Cc1ccccc1)C(=O)O]

### 2-Benzylhexanedioic acid 7

This compound was prepared according to the general procedure B for the preparation of α-substituted hexanedioic acids. 90% yield, (1.28 g, 5.4 mmol, white solid).

¹H NMR (600 MHz, CDCl₃) δ 7.30-7.16 (m, 5H), 3.01 (dd, J=13.6, 7.4 Hz, 1H), 2.78-2.65 (m, 2H), 2.40-2.26 (m, 2H), 1.78-1.61 (m, 3H), 1.59-1.51 (m, 1H).

¹³C NMR (151 MHz, CDCl₃) δ 181.5, 179.5, 138.9, 129.0, 128.6, 126.7, 47.2, 38.1, 33.9, 31.0, 22.5. HRMS (ESI-TOF) Calculated for C₁₃H₁₆O₄Na [M+Na]⁺: 259.0946, Found: 259.0947.

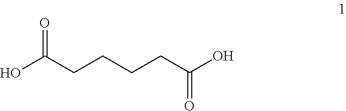

### Adipic Acid/Hexane-1,6-Dioic Acid 1

This compound is commercially available.

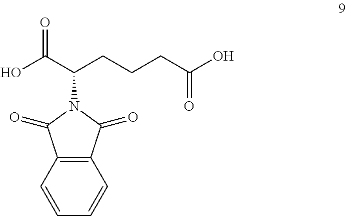 [SMILES: O=C(O)CCC[C@@H](C(=O)O)N1C(=O)c2ccccc2C1=O]

### (S)-2-(1,3-dioxoisoindolin-2-yl)hexanedioic acid 9

This compound was synthesized from commercially available L-homoglutamic acid using the following general procedure(35). Amino acid (5.0 mmol, 1.0 equiv), phthalic anhydride (5.0 mmol, 1.0 equiv), and triethylamine (0.5 mmol, 0.1 equiv) were dissolved in toluene (30 mL) in a dried round bottom flask equipped with a stir bar and a reflux condenser. The reaction mixture was heated to reflux for 4 h. The reaction mixture was cooled to room temperature and concentrated under vacuum. The residue was dissolved in EtOAc, transferred to a separatory funnel, and washed with 1 M HCl. The organic layer was dried with MgSO₄, filtered through Celite, and concentrated to give the desired product quantitatively as a white powder (1.46 g, 5.0 mmol).

¹H NMR (500 MHz, DMSO) δ 8.14-7.74 (m, 4H), 4.74 (dd, J=10.0, 5.4 Hz, 1H), 2.21 (hept, J=8.0 Hz, 2H), 2.09 (ddt, J=14.6, 12.6, 7.4 Hz, 2H), 1.46 (p, J=7.5 Hz, 2H).

¹³C NMR (126 MHz, DMSO) δ 174.1, 170.4, 167.4, 134.9, 131.1, 123.4, 51.4, 32.9, 27.5, 21.4. HRMS (ESI-TOF) Calculated for C₁₄H₁₄NO₆ [M+H]⁺: 292.0821, Found: 292.0821.

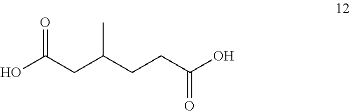

### 3-Methylhexanedioic acid 12

This compound is commercially available.

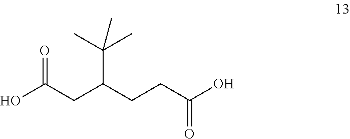

### 3-(tert-Butyl)hexanedioic acid 13

This compound is commercially available.

### 1-β-Carboxypropyl)cyclopropane-1-carboxylic acid 14

This compound was synthesized using the following procedure:

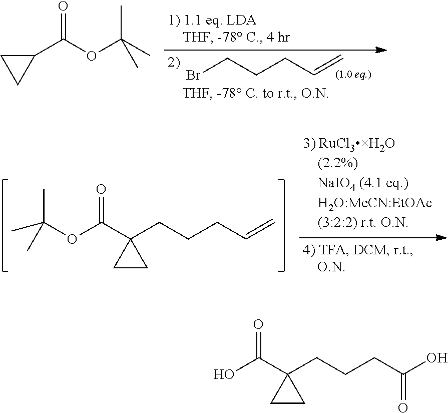

A solution of nBuLi (2.5M in hexanes, 1.4 mL, 3.6 mmol, 1.2 eq.) was added to a solution of DIPA (0.46 mL, 3.3 mmol, 1.1 eq.) in THF (30 mL) at 0° C. The resultant solution was stirred at 0° C. for 30 minutes then cooled to −78° C. tert-Butyl cyclopropanecarboxylate (427 mg, 3.0 mmol, 1.0 eq.) was dissolved in THF (2.0 mL) and added dropwise to the LDA solution at −78° C., in which the solution was stirred at −78° C. for 4 hours. A solution of 5-bromo-1-pentene (0.36 mL, 3.0 mmol, 1.0 eq.) in THF (2.0 mL) was then added. The reaction was warmed to room temperature and stirred overnight. The completion of the reaction was confirmed by TLC analysis of the reaction mixture. The reaction was then quenched with aq. HCl (1.0 M) and extracted three times with EtOAc. The combined organic layer was dried with anhydrous MgSO₄, filtered, and concentrated under vacuum. The crude product obtained was directly used for the next step without further purification.

To the crude product obtained in the previous step was added a 3:2:2 ratio of H₂O, MeCN, and EtOAc (0.3 M) at room temperature, followed by RuCl₃·xH₂O (14 mg, 2.2 mol %). The reaction was stirred vigorously and NaIO₄ (2.7 g, 12.3 mmol, 4.1 eq.) was added in portions over 1 hour. The reaction mixture was then stirred at room temperature overnight. The reaction mixture was then diluted with water and extracted with EtOAc three times. The combined organic layer was dried with anhydrous MgSO₄, filtered, and concentrated under vacuum. To the crude product was added aq. NaOH (10.0 mL, 1.0 M) and quickly extracted with EtOAc three times. The aqueous layer was acidified with aq. HCl (6.0 M) to pH˜2 and extracted with EtOAc three times. The combined organic layer was dried with anhydrous MgSO₄, filtered, and concentrated. The residue was dissolved in DCM (10 mL) and trifluoroacetic acid (0.23 mL, 3.0 mmol, 1.0 eq.) was added and stirred at room temperature overnight. Evaporation of the volatiles provided the desired product as a white solid in 20% yield over 4 steps (103.0 mg, 0.6 mmol).

¹H NMR (500 MHz, DMSO) δ 2.17 (t, J=7.5 Hz, 2H), 1.71-1.57 (m, 2H), 1.54-1.39 (m, 2H), 1.15-0.94 (m, 2H), 0.71-0.56 (m, 2H).

¹³C NMR (126 MHz, DMSO) δ 176.2, 174.3, 33.7, 32.8, 22.9, 22.6, 14.7. HRMS (ESI-TOF) Calculated for C₈H₁₁O₄ [M−H]⁻: 171.0657,

Found: 171.0661.

### 1-β-Carboxypropyl)cyclobutane-1-carboxylic acid 15

This compound was prepared according to the general procedure A for the preparation of α-substituted hexanedioic acids.

Elution condition: (20% EA/hexanes+1% AcOH to 50% EA/hexanes+1% AcOH, gradient elution) Isolated yield: 41% over 2 steps (0.76 g, 4.1 mmol, white solid)

¹H NMR (500 MHz, DMSO) δ 12.05 (s, 2H), 2.32-2.23 (m, 2H), 2.19 (t, J=7.4 Hz, 2H), 1.87-1.71 (m, 4H), 1.71-1.62 (m, 2H), 1.41-1.30 (m, 2H).

¹³C NMR (126 MHz, DMSO) δ 177.8, 174.3, 46.8, 36.9, 33.7, 29.5, 20.2, 15.0. HRMS (ESI-TOF) Calculated for C₉H₁₃O₄ [M−H]⁻: 185.0814,

Found: 185.0818.

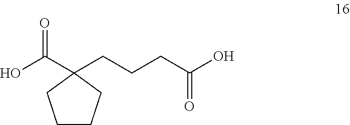 [SMILES: O=C(O)CCCC1(C(=O)O)CCCC1]

### 1-β-Carboxypropyl)cyclopentane-1-carboxylic acid 16

This compound was prepared according to the general procedure A for the preparation of α-substituted hexanedioic acids.

Elution condition: (20% EA/hexanes+1% AcOH to 50% EA/hexanes+1% AcOH, gradient elution) Isolated yield: 55% over 2 steps (1.10 g, 5.5 mmol, white solid)

¹H NMR (500 MHz, DMSO) δ 12.02 (s, 2H), 2.17 (t, J=7.3 Hz, 2H), 2.00 (dt, J=12.0, 6.2 Hz, 2H), 1.69-1.45 (m, 6H), 1.45-1.20 (m, 4H).

¹³C NMR (126 MHz, DMSO) δ 178.5, 174.2, 53.1, 38.1, 35.3, 34.0, 24.6, 21.3. HRMS (ESI-TOF) Calculated for C₁₀H₁₅O₄ [M−H]⁻: 199.0970, Found: 199.0978.

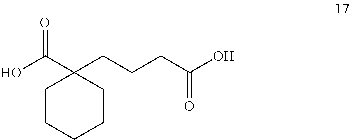 [SMILES: O=C(O)CCCC1(C(=O)O)CCCCC1]

### 1-β-Carboxypropyl)cyclohexane-1-carboxylic acid 17

This compound was prepared according to the general procedure A for the preparation of α-substituted hexanedioic acids.

Elution condition: (20% EA/hexanes+1% AcOH to 50% EA/hexanes+1% AcOH, gradient elution) Isolated yield: 56% over 2 steps (1.20 g, 5.6 mmol, white solid)

¹H NMR (500 MHz, DMSO) δ 12.04 (s, 2H), 2.15 (dq, J=4.4, 2.3 Hz, 2H), 1.92 (d, J=12.2 Hz, 2H), 1.61-1.47 (m, 3H), 1.40 (d, J=3.7 Hz, 4H), 1.27 (dt, J=14.5, 10.3 Hz, 2H), 1.20 (td, J=9.5, 3.5 Hz, 1H), 1.13 (td, J=12.1, 3.1 Hz, 2H).

¹³C NMR (126 MHz, DMSO) δ 177.4, 174.2, 45.8, 39.3, 34.0, 33.6, 25.5, 22.9, 19.4. HRMS (ESI-TOF) Calculated for C₁₁H₁₇O₄ [M−H]⁻:

213.1127, Found: 213.1125.

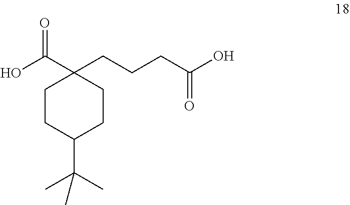 [SMILES: CC(C)(C)C1CCC(CCCC(=O)O)(C(=O)O)CC1]

### 4-(tert-Butyl)-1-β-carboxypropyl)cyclohexane-1-carboxylic acid 18

This compound was prepared according to the general procedure A for the preparation of α-substituted hexanedioic acids.

Elution condition: (10% EA/hexanes+1% AcOH to 50% EA+1% AcOH, gradient elution) Isolated yield: 56% over 2 steps (1.51 g, 5.6 mmol, white solid, d.r.=2.7:1)

¹H NMR (500 MHz, DMSO) δ 12.03 (s, 2H), 2.21-2.07 (m, 3.5H), 1.79-1.71 (m, 0.5H), 1.64-1.48 (m, 2.5H), 1.45-1.31 (m, 4H), 1.16-1.06 (m, 0.6H), 1.06-0.91 (m, 4H), 0.85-0.79 (m, 9H).

¹³C NMR (126 MHz, DMSO) δ 178.8, 177.1, 174.3, 174.2, 47.2, 47.0, 45.9, 43.8, 40.9, 34.3, 34.0, 34.0, 32.1, 32.1, 30.9, 27.3, 24.2, 21.5, 19.8, 19.5.

HRMS (ESI-TOF) Calculated for C₁₅H₂₅O₄ [M−H]⁻: 269.1753, Found: 269.1748.

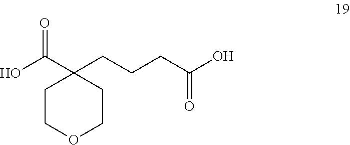 [SMILES: O=C(O)CCCC1(C(=O)O)CCOCC1]

### 4-β-Carboxypropyl)tetrahydro-2H-pyran-4-carboxylic acid 19

This compound was prepared according to the general procedure A for the preparation of α-substituted hexanedioic acids.

Elution condition: (40% EA/hexanes+1% AcOH to 100% EA+1% AcOH, gradient elution) Isolated yield: 70% over 2 steps (1.51 g, 7.0 mmol, white solid)

¹H NMR (500 MHz, DMSO) δ 12.23 (s, 2H), 3.70 (dt, J=11.8, 3.8 Hz, 2H), 3.29 (dd, J=11.4, 2.3 Hz, 2H), 2.17 (t, J=6.9 Hz, 2H), 1.90 (d, J=13.2 Hz, 2H), 1.49-1.43 (m, 2H), 1.43-1.37 (m, 2H), 1.37-1.31 (m, 2H).

¹³C NMR (126 MHz, DMSO) δ 176.7, 174.2, 64.6, 43.8, 39.2, 33.7, 19.1. HRMS (ESI-TOF) Calculated for C₁₀H₁₅₀₅ [M−H]⁻: 215.0919,

Found: 215.0914.

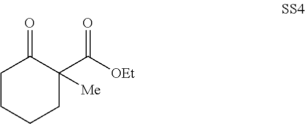 [SMILES: CCOC(=O)C1(C)CCCCC1=O]

### Ethyl 1-methyl-2-oxocyclohexane-1-carboxylate SS4

This compound was prepared according to the general procedure B for the preparation of α-substituted heptanedioic acids with the reaction of MeI and ethyl 2-oxocyclohexane-1-carboxylate. 73% yield, (1.1 g, 6.0 mmol, colorless oil).

¹H NMR (600 MHz, CDCl₃) δ 4.22-4.14 (m, 2H), 2.54-2.41 (m, 3H), 2.06-1.97 (m, 1H), 1.78-1.57 (m, 4H), 1.50-1.41 (m, 1H), 1.28 (s, 3H), 1.25 (t, J=7.1 Hz, 3H).

¹³C NMR (151 MHz, CDCl₃) δ 208.5, 173.2, 61.4, 57.3, 40.8, 38.4, 27.7, 22.8, 21.4, 14.2.

The data is consistent with those reported in the literature.(33)

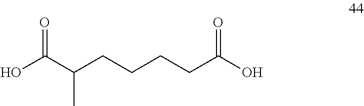

### 2-Methylheptanedioic acid 44

This compound was prepared according to the general procedure B for the preparation of α-substituted heptanedioic acids. 75% yield, (0.78 g, 4.5 mmol, white solid).

¹H NMR (500 MHz, DMSO) δ 2.29 (h, J=6.9 Hz, 1H), 2.19 (t, J=7.3 Hz, 2H), 1.57-1.50 (m, 1H), 1.50-1.41 (m, 2H), 1.33 (dq, J=15.7, 6.8 Hz, 1H), 1.25 (qd, J=7.6, 3.7 Hz, 2H), 1.03 (d, J=6.9 Hz, 3H).

¹³C NMR (126 MHz, DMSO) δ 177.4, 174.4, 38.6, 33.6, 33.0, 26.3, 24.4, 17.0. HRMS (ESI-TOF) Calculated for C₈H₁₃O₄, [M−H]⁻: 173.0814, found: 173.0811.

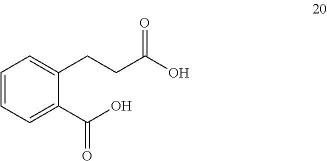 [SMILES: O=C(O)CCc1ccccc1C(=O)O]

### 2-(2-Carboxyethyl)benzoic acid 20

This compound is commercially available.

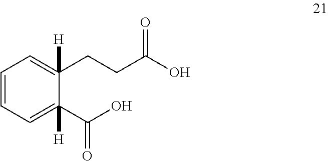

### (1R*,2R*)-2-(2-Carboxyethyl)cyclohexane-1-carboxylic acid 21

This compound was prepared according to the following procedure.(36)

The compound 20 (0.2 g, 1.03 mmol) was dissolved in glacial acetic acid (10.0 mL) and PtO₂ (40.0 mg) was added. The reaction vessel was purged with H₂ and the hydrogenation reaction was carried out at room temperature with a balloon of H₂ and stirred for 3 days. Analysis of an aliquot of the reaction mixture at this point suggested completion of reaction. The reaction mixture was filtered with a plug of Celite® and the filtrate was evaporated to dryness to give the desired compound as a white solid in 97% yield (0.2 g, 1.00 mmol).

¹H NMR (500 MHz, CDCl₃) δ 2.62 (dt, J=8.1, 4.1 Hz, 1H), 2.41 (ddd, J=15.8, 10.0, 5.8 Hz, 1H), 2.32 (ddd, J=16.0, 9.7, 6.4 Hz, 1H), 1.98-1.85 (m, 1H), 1.83-1.73 (m, 2H), 1.73-1.61 (m, 4H), 1.60-1.43 (m, 2H), 1.41-1.30 (m, 2H).

¹³C NMR (126 MHz, CDCl₃) δ 181.6, 180.4, 45.2, 36.7, 32.6, 28.1, 25.3, 23.9, 22.4. HRMS (ESI-TOF) Calculated for C₁₀H₁₅O₄ [M−H]⁻: 199.0970, Found: 199.0968.

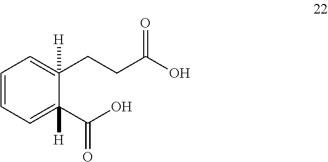

### (1R*,2S*)-2-(2-Carboxyethyl)cyclohexane-1-carboxylic acid 22

This compound was prepared according to the following procedure.(36)

The compound 21 (0.1 g, 0.50 mmol) was suspended in conc. HCl (1.0 mL) in a sealed tube and stirred at 180° C. overnight. The reaction mixture was diluted with H₂O and extracted with EtOAc three times. The combined organic layer was dried with anhydrous MgSO₄, filtered, and evaporated to dryness to give the desired compound as an off-white solid in 76% yield (75.0 mg, 0.38 mmol).

¹H NMR (500 MHz, CDCl₃) δ 2.48 (ddd, J=16.2, 10.8, 5.5 Hz, 1H), 2.28 (ddd, J=16.1, 10.5, 5.7 Hz, 1H), 2.08 (td, J=11.5, 3.5 Hz, 1H), 1.96 (d, J=13.7 Hz, 1H), 1.89-1.79 (m, 2H), 1.79-1.72 (m, 2H), 1.66-1.56 (m, 1H), 1.53-1.41 (m, 2H), 1.30-1.19 (m, 2H), 0.92 (td, J=12.3, 8.9 Hz, 1H).

¹³C NMR (126 MHz, CDCl₃) δ 182.9, 180.6, 49.9, 38.3, 31.5, 30.4, 30.1, 29.8, 25.6, 25.4. HRMS (ESI-TOF) Calculated for C₁₀H₁₅O₄ [M−H]⁻: 199.0970, Found: 199.0974.

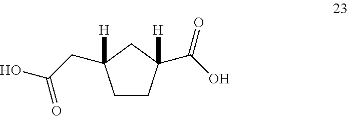 [SMILES: O=C(O)C[C@H]1CC[C@@H](C(=O)O)C1]

### (1R*,3S*)-3-(Carboxymethyl)cyclopentane-1-carboxylic acid 23

This compound was prepared according to the following procedure.

Bicyclo[3.2.1]oct-2-ene (1.0 g, 9.24 mmol) was dissolved in a 3:2:2 ratio of H₂O/MeCN/EtOAc (31 mL) at room temperature. RuCl₃·xH₂O (42.0 mg, 0.20 mmol) was added, followed by portionwise addition of NaIO₄ (8.1 g, 38.0 mmol) over 1 hour. The reaction mixture was stirred vigorously at room temperature overnight. The reaction mixture was diluted with H₂O and extracted with EtOAc three times. The combined organic layer was dried with anhydrous MgSO₄, filtered, and evaporated to dryness to give the desired compound as a pale grey solid in 83% yield (1.35 g, 7.6 mmol).

¹H NMR (600 MHz, DMSO) δ 12.01 (s, 2H), 2.69 (dt, J=16.4, 8.1 Hz, 1H), 2.24 (dd, J=7.3, 2.0 Hz, 2H), 2.19-2.10 (m, 1H), 2.03 (dt, J=12.5, 7.5 Hz, 1H), 1.83-1.71 (m, 3H), 1.31 (dt, J=12.5, 9.5 Hz, 1H), 1.24-1.18 (m, 1H).

¹³C NMR (151 MHz, DMSO) δ 177.1, 173.9, 42.9, 39.4, 36.2, 36.0, 31.4, 28.3. HRMS (ESI-TOF) Calculated for C₈H₁₁O₄ [M−H]⁻: 171.0657,

Found: 171.0657.

### General Method for the Preparation of Substituted Benzoic Acid Substrates

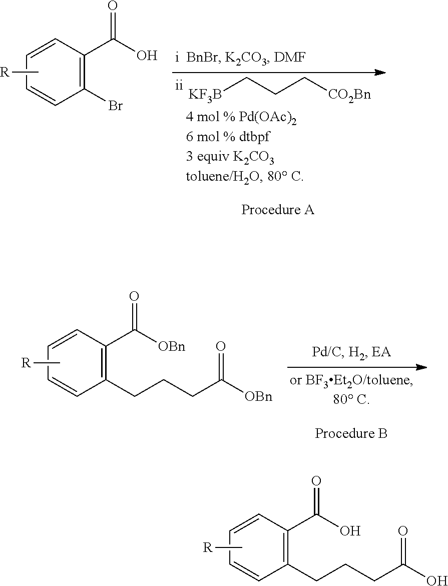

### Procedure A

To a solution of 2-bromobenzoic acid (14.0 mmol) in DMF (30 mL) were added K₂CO₃ (21.0 mmol) and benzylbromide (14.3 mmol), and the mixture was stirred at room temperature for 12 h. The mixture was partitioned between EtOAc and H₂O, and extracted with EtOAc, and the organic layer was washed with H₂O and brine, dried over MgSO₄, filtered, and concentrated in vacuo to give benzyl 2-bromobenzoate quantitatively.

A suspension of benzyl 2-bromobenzoate (2 mmol), benzyl 4-trifluoroboratebutanoate potassium salt (3 mmol), K₂CO₃ (829 mg, 6 mmol), Pd(OAc)₂ (18 mg, 0.08 mmol), 1,1′-bis(di-tert-butylphosphino)ferrocene (dtbpf, 56 mg, 0.12 mmol) in toluene (10 mL), was degassed with N₂ during 5 min and distilled water (2 mL) was added. The mixture was placed into a pre-heated oil bath at 80° C. during 18 h and controlled by TLC until the starting material was consumed. Then, the system was cooled to room temperature, diluted with H₂O (10 mL) and extracted with Et₂O (3×30 mL). The organic phase was washed with 1 M HCl (10 mL) and brine (20 mL), dried over MgSO₄ and filtered through a cotton pad. The volatiles were removed under vacuum and the crude material was purified by column chromatography affording benzyl 2-(4-(benzyloxy)-4-oxobutyl)benzoate.

### Preparation of benzyl 4-trifluoroboratebutanoate potassium salt

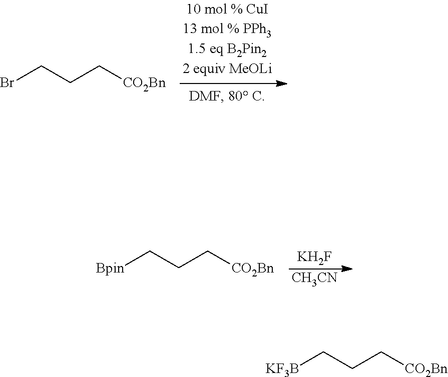

In air, CuI (228 mg, 1.2 mmol), PPh₃ (409 mg, 1.56 mmol), LiOMe (912 mg, 24 mmol), and bis(pinacolato)diboron (4.57 g, 18 mmol) were added to a 50 ml round-bottom flask equipped with a stir bar. The vessel was evacuated and filled with N₂ (three cycles). DMF (15 mL), the benzyl 4-bromobutanoate (12 mmol, 3.07 g) were added in turn by syringe. The resulting reaction mixture was stirred vigorously at room temperature for 18 h. The reaction mixture was then diluted with EtOAc, filtered through silica gel with copious washings (EtOAc), concentrated, and purified by column chromatography, affording benzyl 4-(4,4,5,5-tetramethyl-1,3,2-dioxaborolan-2-yl)butanoate (2.9 g, 79% yield) as a colorless oil. ^{L}H NMR (600 MHz, CDCl₃) δ 7.39-7.28 (m, 5H), 5.11 (s, 2H), 2.41-2.35 (m, 2H), 1.81-1.73 (m, 2H), 1.23 (s, 12H), 0.82 (t, J=7.9 Hz, 2H). ¹³C NMR (151 MHz, CDCl₃) δ 173.6, 136.3, 128.6, 128.3, 128.2, 83.2, 66.1, 36.7, 24.9, 19.7. HRMS (ESI-TOF) Calculated for C₂₅H₂₅O₄ [M+H]⁺: 304.1960, found: 304.1963.

To a stirred solution of benzyl 4-(4,4,5,5-tetramethyl-1,3,2-dioxaborolan-2-yl)butanoate (1.2 g, 4 mmol) in CH₃CN (10 mL) was added KHF₂ (936 mg, 12 mmol) Then the mixture was added with water (3 mL) dropwise at ambient temperature and the reaction mixture was stirred for 4 h. Then the solvent was removed under vacuum and dried. The resulting mixture was purified by dissolving in hot CH₃CN, and precipitation by adding Et₂O. Collect the white solid washed with Et₂O to afford benzyl 4-trifluoroboratebutanoate potassium salt (795 mg, 70% yield) as a white solid. ¹H NMR (600 MHz, DMSO) δ 7.60-7.16 (m, 5H), 5.04 (s, 2H), 2.33-2.03 (m, 2H), 1.83-1.32 (m, 2H), 0.40--0.18 (m, 2H). ¹³C NMR (151 MHz, DMSO) δ 173.8, 136.6, 128.4, 127.9, 127.8, 73.6, 64.8, 37.3, 25.0, 21.7 (q, J=2.7 Hz). HRMS (ESI-TOF) Calculated for C₁₁H₁₃BF₃O₂ [M−H]⁻: 244.0997, found: 244.0992.

### Procedure B

A solution of benzyl 2-(4-(benzyloxy)-4-oxobutyl)benzoate (1 mmol) in EtOAc (10 mL) was treated with 10% Pd/C (100 mg) and the mixture was stirred for overnight at room temperature under H₂. The mixture was passed through celite and the filtrate was concentrated under reduced pressure to give 2-β-carboxypropyl)benzoic acid.

### Procedure C

A solution of benzyl 2-(4-(benzyloxy)-4-oxobutyl)benzoate (1 mmol) in toluene (10 mL) was added with Boron trifluoride diethyl etherate (2.4 mmol) and the mixture was placed into a pre-heated oil bath at 80° C. for 2 h. The mixture was concentrated under reduced pressure and purified by column chromatography (EtOAc/Hexanes/3% AcOH) to give 2-β-carboxypropyl)benzoic acid.

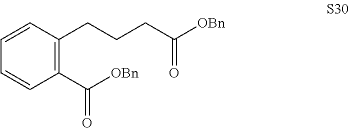 [SMILES: O=C(CCCc1ccccc1C(=O)OCc1ccccc1)OCc1ccccc1]

### Benzyl 2-(4-(benzyloxy)-4-oxobutyl)benzoate S30

S30 was prepared by Procedure A, 97% yield for two steps, (0.75 g, 1.94 mmol, yellow oil).

¹H NMR (600 MHz, CDCl₃) δ 7.94-7.90 (m, 1H), 7.44-7.31 (m, 11H), 7.26-7.19 (m, 2H), 5.32 (s, 2H), 5.11 (s, 2H), 3.00-2.95 (m, 2H), 2.34 (t, J=7.5 Hz, 2H), 1.97-1.89 (m, 2H).

¹³C NMR (151 MHz, CDCl₃) δ 173.4, 167.4, 143.6, 136.2, 136.1, 132.2, 131.3, 131.0, 129.5, 128.8, 128.7, 128.5, 128.4, 128.4, 128.3, 128.3, 126.3, 77.4, 77.2, 76.9, 66.8, 66.27, 34.02, 33.7, 26.8.

HRMS (ESI-TOF) Calculated for C₂₅H₂₅O₄ [M+H]⁺: 389.1753, Found: 389.1749.

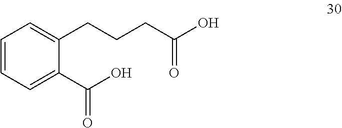 [SMILES: O=C(O)CCCc1ccccc1C(=O)O]

### 2-β-Carboxypropyl)benzoic acid 30

30 was prepared by Procedure B, 99% yield, (0.21 g, 0.99 mmol, white solid).

¹H NMR (600 MHz, CDCl₃) δ 8.00 (dd, J=8.1, 1.5 Hz, 1H), 7.49 (td, J=7.5, 1.5 Hz, 1H), 7.33-7.27 (m, 2H), 3.18-3.12 (m, 2H), 2.43-2.38 (m, 2H), 2.05-1.98 (m, 2H).

¹³C NMR (151 MHz, CDCl₃) δ 181.1, 174.3, 144.1, 133.0, 131.7, 131.3, 129.2, 126.4, 77.4, 77.2, 77.0, 33.4, 32.6, 27.7.

HRMS (ESI-TOF) Calculated for C₁₁H₁₂O₄Na [M+Na]⁺: 231.0633, Found: 231.0640.

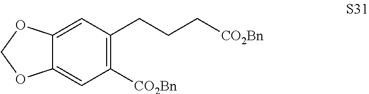 [SMILES: O=C(CCCc1cc2c(cc1C(=O)OCc1ccccc1)OCO2)OCc1ccccc1]

### Benzyl 6-(4-(benzyloxy)-4-oxobutyl)benzo[d][1,3]dioxole-5-carboxylate S31

S31 was prepared by Procedure A, 95% yield for two steps, (0.82 g, 1.90 mmol, yellow oil).

¹H NMR (600 MHz, CDCl₃) δ 7.43-7.32 (m, 10H), 6.66 (s, 1H), 5.98 (s, 2H), 5.28 (s, 2H), 5.11 (s, 2H), 2.97-2.92 (m, 2H), 2.35 (t, 2H), 1.95-1.87 (m, 2H).

¹³C NMR (151 MHz, CDCl₃) δ 173.4, 166.3, 150.9, 146.0, 140.6, 136.2, 136.2, 128.7, 128.7, 128.4, 128.4, 128.3, 122.2, 111.0, 110.8, 101.8, 66.7, 66.3, 33.9, 33.9, 26.9.

HRMS (ESI-TOF) Calculated for C₂₆H₂₅O₆ [M+H]⁺: 433.1651, Found: 433.1646.

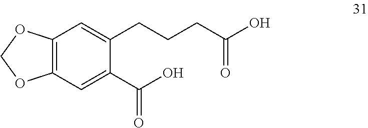 [SMILES: O=C(O)CCCc1cc2c(cc1C(=O)O)OCO2]

### 6-β-Carboxypropyl)benzo[d][1,3]dioxole-5-carboxylic acid 31

31 was prepared by Procedure B, 99% yield, (0.25 g, 0.99 mmol, white solid).

¹H NMR (600 MHz, CD₃OD) δ 7.37 (s, 1H), 6.76 (s, 1H), 6.00 (s, 2H), 3.00-2.95 (m, 2H), 2.31 (t, J=7.6 Hz, 2H), 1.90-1.82 (m, 2H).

¹³C NMR (151 MHz, CD₃OD) δ 177.4, 170.0, 152.2, 147.3, 141.6, 124.0, 111.8, 111.5, 103.1, 34.7, 34.6, 28.2.

HRMS (ESI-TOF) Calculated for C₁₂H₁₂O₆Na [M+Na]⁺: 275.0532, Found: 275.0530.

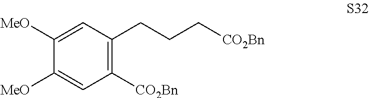 [SMILES: COc1cc(CCCC(=O)OCc2ccccc2)c(C(=O)OCc2ccccc2)cc1OC]

### Benzyl 2-(4-(benzyloxy)-4-oxobutyl)-4,5-dimethoxybenzoate S32

S32 was prepared by Procedure A, 95% yield for two steps, (0.85 g, 1.90 mmol, yellow oil).

¹H NMR (600 MHz, CDCl₃) δ 7.50 (s, 1H), 7.44-7.41 (m, 2H), 7.40-7.30 (m, 9H), 6.66 (s, 1H), 5.31 (s, 2H), 5.11 (s, 2H), 3.87 (s, 3H), 3.87 (s, 3H), 2.98-2.92 (m, 2H), 2.31 (t, J=7.4 Hz, 2H), 1.95-1.87 (m, 2H).

¹³C NMR (151 MHz, CDCl₃) δ 173.5, 166.8, 152.1, 146.9, 138.5, 136.3, 136.2, 128.7, 128.7, 128.4, 128.4, 128.4, 128.3, 120.8, 113.9, 113.8, 66.7, 66.3, 56.2, 56.0, 33.9, 33.8, 27.0. HRMS (ESI-TOF) Calculated for C₂₇H₂₉O₆ [M+H]⁺: 449.1964, Found: 449.1956.

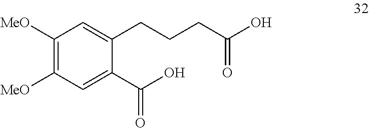 [SMILES: COc1cc(CCCC(=O)O)c(C(=O)O)cc1OC]

### 2-β-Carboxypropyl)-4,5-dimethoxybenzoic acid 32

32 was prepared by Procedure B, 99% yield, (0.27 g, 0.99 mmol, white solid).

¹H NMR (600 MHz, CD₃OD) δ 7.52 (s, 1H), 6.84 (s, 1H), 3.88 (s, 3H), 3.84 (s, 3H), 3.04-2.98 (m, 2H), 2.32 (t, J=7.4 Hz, 2H), 1.93-1.85 (m, 2H).

¹³C NMR (151 M1 Hz, CD₃OD) δ 177.8, 170.7, 153.5, 148.1, 139.9, 122.7, 115.7, 115.2, 34.75, 34.2, 28.2.

HRMS (ESI-TOF) Calculated for C₁₃H₁₅O₆ [M−H]⁻: 267.0869, Found: 267.0858.

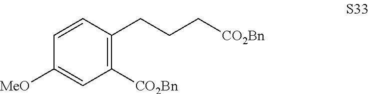 [SMILES: COc1ccc(CCCC(=O)OCc2ccccc2)c(C(=O)OCc2ccccc2)c1]

### Benzyl 2-(4-(benzyloxy)-4-oxobutyl)-4-methoxybenzoate S33

S33 was prepared by Procedure A, 74% yield for two steps, (0.62 g, 1.48 mmol, yellow oil).

¹H NMR (600 MHz, CDCl₃) δ 7.47-7.41 (m, 3H), 7.40-7.30 (m, 8H), 7.12 (d, J=8.5 Hz, 1H), 6.97 (dd, J=8.4, 2.9 Hz, 1H), 5.33 (s, 2H), 5.11 (s, 2H), 3.80 (s, 3H), 2.93-2.87 (m, 2H), 2.34-2.28 (m, 2H), 1.94-1.86 (m, 2H).

¹³C NMR (151 MHz, CDCl₃) δ 173.5, 167.2, 157.7, 136.2, 136.0, 135.6, 132.4, 130.3, 128.8, 128.7, 128.5, 128.4, 128.4, 128.3, 118.3, 115.8, 115.8, 66.9, 66.2, 55.6, 33.9, 33.0, 27.0. HRMS (ESI-TOF) Calculated for C₂₆H₂₇O₅ [M+H]⁺: 419.1858, Found: 419.1860.

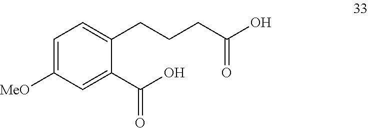 [SMILES: COc1ccc(CCCC(=O)O)c(C(=O)O)c1]

### 2-β-Carboxypropyl)-5-methoxybenzoic acid 33

33 was prepared by Procedure B, 99% yield, (0.24 g, 0.99 mmol, white solid).

¹H NMR (600 MHz, CD₃OD) δ 7.41 (d, J=3.0 Hz, 1H), 7.20 (d, J=8.5 Hz, 1H), 7.03 (dd, J=8.5, 3.0 Hz, 1H), 3.80 (s, 3H), 2.97-2.91 (m, 2H), 2.32-2.27 (m, 2H), 1.90-1.82 (m, 2H).

¹³C NMR (151 MHz, CD₃OD) δ 177.5, 170.9, 159.2, 136.5, 133.3, 132.3, 118.8, 116.6, 55.82, 34.7, 33.8, 28.3.

HRMS (ESI-TOF) Calculated for C₁₂H₁₃O₅ [M−H]⁻: 237.0763, found: 237.0766.

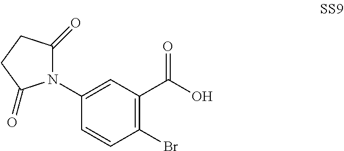 [SMILES: O=C(O)c1cc(N2C(=O)CCC2=O)ccc1Br]

### 2-Bromo-4-(2,5-dioxopyrrolidin-1-yl)benzoic acid SS9

The synthesis of 2-bromo-4-(2,5-dioxopyrrolidin-1-yl)benzoic acid SS9: A mixture of 864 mg (4 mmol) of 4-amino-2-bromobenzoic acid, 1 g (4 mmol) of succinic anhydride, 0.6 ml of ClSiMe₃, and 1.5 ml DMF was heated for 1.5 h on a bath at 160-165° C. cooled to room temperature, and diluted with water. The precipitate was separated by filtration, washed with water, and dried in air to obtain 2-bromo-4-(2,5-dioxopyrrolidin-1-yl)benzoic acid (90% yield, 1.07 g, 3.6 mmol) as yellow solid.

¹H NMR (600 MHz, CD₃OD) δ 7.85-7.80 (m, 2H), 7.40 (dd, J=8.5, 2.6 Hz, 1H), 2.88 (s, 4H).

¹³C NMR (151 MHz, CD₃OD) δ 178.4, 168.5, 135.8, 135.1, 131.8, 130.6, 121.6, 29.4. HRMS (ESI-TOF) Calculated for C₁₁H₉BrNO₄ [M+H]⁺: 297.9715, Found: 297.9716.

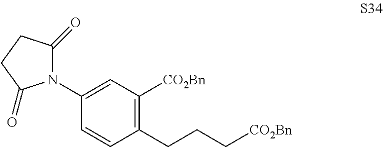 [SMILES: O=C(CCCc1ccc(N2C(=O)CCC2=O)cc1C(=O)OCc1ccccc1)OCc1ccccc1]

### Benzyl 2-(4-(benzyloxy)-4-oxobutyl)-4-(2,5-dioxopyrrolidin-1-yl)benzoate S34

S34 was prepared by Procedure A, 77% yield for two steps, (0.75 g, 1.54 mmol, yellow oil).

¹H NMR (600 MHz, CDCl₃) δ 7.88 (dd, J=2.0, 0.8 Hz, 1H), 7.44-7.30 (m, 12H), 5.31 (s, 2H), 5.12 (s, 2H), 3.03-2.99 (m, 2H), 2.88 (s, 4H), 2.36 (t, J=7.4 Hz, 2H), 1.98-1.90 (m, 2H).

¹³C NMR (151 MHz, CDCl₃) δ 176.1, 173.3, 166.1, 144.5, 136.2, 135.8, 132.3, 130.3, 130.1, 130.1, 129.2, 128.8, 128.8, 128.6, 128.6, 128.5, 128.4, 67.1, 66.3, 34.0, 33.5, 28.5, 26.7. HRMS (ESI-TOF) Calculated for C₂₉H₂₈NO₆ [M+H]⁺: 486.1917, Found: 486.1910.

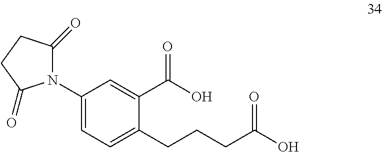 [SMILES: O=C(O)CCCc1ccc(N2C(=O)CCC2=O)cc1C(=O)O]

### 2-β-Carboxypropyl)-4-(2,5-dioxopyrrolidin-1-yl)benzoic acid 34

34 was prepared by Method B, 98% yield, (0.3 g, 0.98 mmol, white solid).

¹H NMR (600 MHz, CD₃OD) δ 7.86 (d, J=2.2 Hz, 1H), 7.44-7.37 (m, 2H), 3.10-3.04 (m, 2H), 2.85 (s, 4H), 2.35 (t, J=7.5 Hz, 2H), 1.97-1.89 (m, 2H).

¹³C NMR (151 MHz, CD₃OD) δ 177.6, 175.9, 168.5, 143.7, 131.5, 130.7, 130.5, 129.9, 129.1, 33.2, 32.9, 28.0, 26.6.

HRMS (ESI-TOF) Calculated for C₁₅H₁₆NO₆ [M+H]⁺: 306.0978, Found: 306.0965.

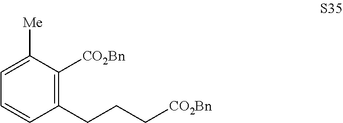 [SMILES: Cc1cccc(CCCC(=O)OCc2ccccc2)c1C(=O)OCc1ccccc1]

### Benzyl 2-(4-(benzyloxy)-4-oxobutyl)-3-methylbenzoate S35

S35 was prepared by Procedure A, 83% yield for two steps, (0.67 g, 1.66 mmol, yellow oil).

¹H NMR (600 MHz, CDCl₃) δ 7.47-7.41 (m, 2H), 7.40-7.30 (m, 8H), 7.20 (t, J=7.6 Hz, 1H), 7.05-6.98 (m, 2H), 5.35 (s, 2H), 5.10 (s, 2H), 2.58-2.53 (m, 2H), 2.27 (s, 3H), 2.24 (t, J=7.5 Hz, 2H), 1.90-1.82 (m, 2H)

¹³C NMR (151 MHz, CDCl₃) δ 173.2, 169.8, 138.5, 136.2, 135.6, 135.1, 133.7, 129.6, 129.1, 128.7, 128.7, 128.6, 128.4, 128.3, 128.1, 127.0, 67.1, 66.3, 33.8, 33.0, 26.5, 19.8. HRMS (ESI-TOF) Calculated for C₂₆H₂₇O₄ [M+H]⁺: 403.1909,

Found: 403.1906.

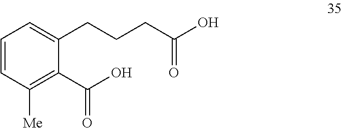 [SMILES: Cc1cccc(CCCC(=O)O)c1C(=O)O]

### 2-β-Carboxypropyl)-6-methylbenzoic acid 35

35 was prepared by Procedure B, 99% yield, (0.22 g, 0.99 mmol, white solid).

¹H NMR (600 MHz, CD₃OD) δ 7.23 (t, J=7.6 Hz, 1H), 7.12-7.06 (m, 2H), 2.71-2.66 (m, 2H), 2.33 (s, 3H), 2.30 (t, J=7.4 Hz, 2H), 1.95-1.87 (m, 2H).

¹³C NMR (151 MHz, CD₃OD) δ 177.2, 173.8, 139.1, 136.3, 135.3, 130.1, 128.9, 127.9, 34.5, 33.9, 27.8, 19.8.

HRMS (ESI-TOF) Calculated for C₁₂H₁₄O₄Na [M+Na]⁺: 245.0790, Found: 245.0795.

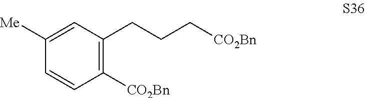 [SMILES: Cc1ccc(C(=O)OCc2ccccc2)c(CCCC(=O)OCc2ccccc2)c1]

### Benzyl 2-(4-(benzyloxy)-4-oxobutyl)-4-methylbenzoate S36

S36 was prepared by Procedure A, 95% yield for two steps, (0.77 g, 1.90 mmol, yellow oil).

¹H NMR (600 MHz, CDCl₃) δ 7.85 (d, J=8.0 Hz, 1H), 7.45-7.40 (m, 2H), 7.37 (dd, J=7.3, 1.0 Hz, 2H), 7.37-7.29 (m, 6H), 7.07-7.00 (m, 2H), 5.30 (s, 2H), 5.11 (s, 2H), 2.98-2.93 (m, 2H), 2.37-2.31 (m, 5H), 1.97-1.88 (m, 2H).

¹³C NMR (151 M1 Hz, CDCl₃) δ 173.5, 167.3, 143.9, 142.8, 136.3, 132.1, 131.3, 128.7, 128.7, 128.4, 128.4, 128.3, 128.3, 127.0, 126.5, 66.6, 66.3, 34.1, 33.8, 26.9, 21.6.

HRMS (ESI-TOF) Calculated for C₂₆H₂₇O₄ [M+H]⁺: 403.1909, Found: 403.1914.

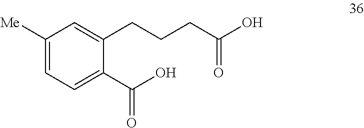 [SMILES: Cc1ccc(C(=O)O)c(CCCC(=O)O)c1]

### 2-β-Carboxypropyl)-4-methylbenzoic acid 36

36 was prepared by Procedure B, 99% yield, (0.22 g, 0.99 mmol, white solid).

¹H NMR (600 MHz, CD₃OD) δ 7.83-7.78 (m, 1H), 7.13-7.07 (m, 2H), 3.02-2.96 (m, 2H), 2.37-2.29 (m, 5H), 1.92-1.84 (m, 2H).

¹³C NMR (151 MHz, CD₃OD) δ 177.4, 170.9, 145.0, 143.8, 132.9, 132.3, 128.2, 127.8, 34.7, 34.7, 28.1, 25.0, 21.4.

HRMS (ESI-TOF) Calculated for C₁₂H₁₄O₄Na [M+Na]⁺: 245.0790, Found: 245.0789.

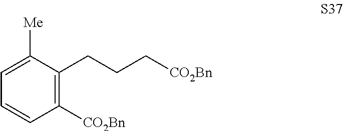 [SMILES: Cc1cccc(C(=O)OCc2ccccc2)c1CCCC(=O)OCc1ccccc1]

### Benzyl 2-(4-(benzyloxy)-4-oxobutyl)-3-methylbenzoate S37

S37 was prepared by Procedure A, 98% yield for two steps, (0.79 g, 1.96 mmol, yellow oil).

¹H NMR (600 MHz, CDCl₃) δ 7.70-7.65 (m, 1H), 7.45-7.40 (m, 2H), 7.38-7.30 (m, 8H), 7.29-7.26 (m, 1H), 7.13 (t, J=7.6 Hz, 1H), 5.31 (s, 2H), 5.12 (s, 2H), 2.93-2.87 (m, 2H), 2.37 (t, J=7.3 Hz, 2H), 2.33 (s, 3H), 1.90-1.81 (m, 2H).

¹³C NMR (151 MHz, CDCl₃) δ 173.4, 168.2, 141.3, 137.7, 136.2, 136.1, 134.1, 130.7, 128.7, 128.7, 128.5, 128.4, 128.4, 128.3, 127.9, 125.9, 125.9, 66.9, 66.3, 34.4, 29.4, 25.4, 19.8. HRMS (ESI-TOF) Calculated for C₂₆H₂₆O₄Na [M+Na]⁺: 425.1729,

Found: 425.1721.

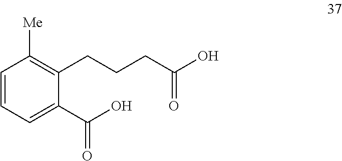 [SMILES: Cc1cccc(C(=O)O)c1CCCC(=O)O]

### 2-β-Carboxypropyl)-3-methylbenzoic acid 37

37 was prepared by Procedure B, 99% yield, (0.22 g, 0.99 mmol, white solid).

¹H NMR (600 MHz, CD₃OD) δ 7.62 (dd, J=7.9, 1.4 Hz, 1H), 7.31 (dt, J=7.6, 1.4 Hz, 1H), 7.14 (t, J=7.6 Hz, 1H), 2.99-2.94 (m, 2H), 2.41-2.35 (m, 5H), 1.88-1.79 (m, 2H).

¹³C NMR (151 MHz, CD₃OD) δ 177.4, 172.2, 142.0, 138.7, 134.6, 132.8, 129.1, 126.7, 35.1, 30.5, 30.4, 26.8, 19.8.

HRMS (ESI-TOF) Calculated for C₁₂H₁₄O₄Na [M+Na]⁺: 245.0790, Found: 245.0798.

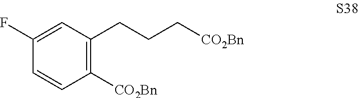 [SMILES: O=C(CCCc1cc(F)ccc1C(=O)OCc1ccccc1)OCc1ccccc1]

### Benzyl 2-(4-(benzyloxy)-4-oxobutyl)-4-fluorobenzoate S38

S38 was prepared by Procedure A, 98% yield for two steps, (0.80 g, 1.96 mmol, yellow oil).

¹H NMR (600 MHz, CDCl₃) δ 8.01-7.95 (m, 1H), 7.45-7.40 (m, 2H), 7.40-7.37 (m, 3H), 7.37-7.30 (m, 5H), 6.96-6.90 (m, 2H), 5.31 (s, 2H), 5.12 (s, 2H), 3.02-2.97 (m, 2H), 2.35 (t, J=7.4 Hz, 2H), 1.98-1.90 (m, 2H).

¹³C NMR (151 MHz, CDCl₃) δ 173.2, 166.3, 164.9 (d, J=253.6 Hz), 147.4 (d, J=8.8 Hz), 136.1 (d, J=29.7 Hz), 133.8 (d, J=9.3 Hz), 128.8, 128.7, 128.5, 128.4, 128.4, 125.5 (d, J=2.8 Hz), 117.9 (d, J=21.4 Hz), 113.4 (d, J=21.4 Hz), 66.9, 66.3, 33.9, 33.8, 26.5.

¹⁹F NMR (376 MHz, CDCl₃) δ −109.70.

HRMS (ESI-TOF) Calculated for C₂₅H₂₄O₄F [M+H]⁺: 407.1659, Found: 407.1653.

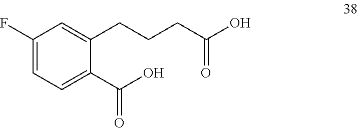 [SMILES: O=C(O)CCCc1cc(F)ccc1C(=O)O]

### 2-β-Carboxypropyl)-4-fluorobenzoic acid 38

38 was prepared by Procedure B, 98% yield, (0.22 g, 0.98 mmol, white solid).

¹H NMR (600 MHz, CD₃OD) δ 7.98 (dd, J=8.7, 6.0 Hz, 1H), 7.08-6.98 (m, 2H), 3.07-3.01 (m, 2H), 2.33 (t, J=7.5 Hz, 2H), 1.94-1.86 (m, 2H).

¹³C NMR (151 MHz, CD₃OD) δ 177.2, 169.8, 166.0 (d, J=251.4 Hz), 148.7 (d, J=8.6 Hz), 134.9 (d, J=9.3 Hz), 127.5 (d, J=3.0 Hz), 118.6 (d, J=21.6 Hz), 114.0 (d, J=21.8 Hz), 34.6, 34.6, 27.8.

¹⁹F NMR (376 MHz, CD₃OD) 5-108.88.

HRMS (ESI-TOF) Calculated for C₁₁H₁₁O₄FNa [M+Na]⁺: 249.0539, Found: 249.0537.

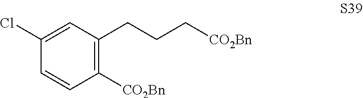 [SMILES: O=C(CCCc1cc(Cl)ccc1C(=O)OCc1ccccc1)OCc1ccccc1]

### Benzyl 2-(4-(benzyloxy)-4-oxobutyl)-4-chlorobenzoate S39

S39 was prepared by Procedure A by using 2-bromo-4-chlorobenzoic acid (3 mmol) with other condition no change, 75% yield, (0.63 g, 1.50 mmol, yellow oil).

¹H NMR (600 MHz, CDCl₃) δ 7.90-7.84 (m, 1H), 7.44-7.30 (m, 10H), 7.24-7.19 (m, 2H), 5.31 (s, 2H), 5.12 (s, 2H), 2.98-2.93 (m, 2H), 2.34 (t, J=7.4 Hz, 2H), 1.96-1.88 (m, 2H).

¹³C NMR (151 MHz, CDCl₃) δ 173.2, 166.5, 145.8, 138.4, 136.2, 131.2, 128.8, 128.7, 128.6, 128.5, 128.4, 127.8, 126.5, 67.0, 66.4, 33.9, 33.6, 26.6.

HRMS (ESI-TOF) Calculated for C₂₅H₂₄ClO₄ [M+H]⁺: 423.1363, Found: 423.1356.

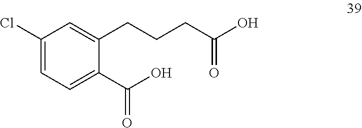 [SMILES: O=C(O)CCCc1cc(Cl)ccc1C(=O)O]

### 2-β-Carboxypropyl)-4-chlorobenzoic acid 39

39 was prepared by Procedure C, 75% yield, (0.18 g, 0.75 mmol, white solid).

¹H NMR (600 MHz, CD₃OD) δ 7.88 (d, J=8.3 Hz, 1H), 7.33 (d, J=2.2 Hz, 1H), 7.29 (dd, J=8.4, 2.2 Hz, 1H), 3.04-2.98 (m, 2H), 2.33 (t, J=7.4 Hz, 2H), 1.93-1.85 (m, 2H).

¹³C NMR (151 MHz, CD₃OD) δ 177.2, 169.9, 147.1, 138.8, 133.7, 131.9, 127.3, 34.6, 34.4, 27.9. HRMS (ESI-TOF) Calculated for C₁₁H₁₂ClO₄ [M+H]⁺: 243.0424, Found: 243.0424.

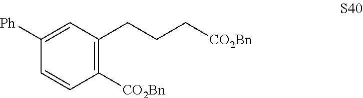 [SMILES: O=C(CCCc1cc(-c2ccccc2)ccc1C(=O)OCc1ccccc1)OCc1ccccc1]

### Benzyl 3-(4-(benzyloxy)-4-oxobutyl)-[1,1′-biphenyl]-4-carboxylate S40

To a solution of Pd₂(dba)₃ (183 mg, 0.2 mmol), SPhos (164 mg, 0.4 mmol) in dioxane (8 mL) was added a solution of S39 (844 mg, 2 mmol) in dioxane (8 mL) and stirred for 10 minutes. K₃PO₄ (1.27 g, 6 mmol) and PhB(OH)₂ were added. The resulting mixture was heated to 100° C. and stirred for 15 hours. The reaction was cooled to room temperature and diluted with ethyl acetate. Then washed with brine and the organic phase was concentrated under reduced pressure and purified by column chromatography to give the product S40 (85% yield, 0.79 g, 1.7 mmol) as a yellow oil.

¹H NMR (600 MHz, CDCl₃) δ 8.03 (d, J=8.1 Hz, 1H), 7.61-7.56 (m, 2H), 7.49-7.42 (m, 6H), 7.41-7.29 (m, 10H), 5.35 (s, 2H), 5.11 (s, 2H), 3.09-3.04 (m, 2H), 2.38 (t, J=7.5 Hz, 2H), 2.03-1.95 (m, 2H).

¹³C NMR (151 MHz, CDCl₃) δ 173.4, 167.1, 145.0, 144.3, 140.0, 136.2, 136.1, 131.8, 130.0, 129.0, 128.8, 128.7, 128.5, 128.4, 128.4, 128.3, 128.2, 128.1, 128.0, 127.4, 124.9, 66.8, 66.3, 34.0, 34.0, 26.9. HRMS (ESI-TOF) Calculated for C₃₁H₂₉O₄ [M+H]⁺: 465.2066, Found: 465.2062.

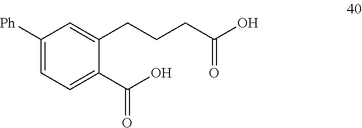 [SMILES: O=C(O)CCCc1cc(-c2ccccc2)ccc1C(=O)O]

### 3-β-Carboxypropyl)-[1,1′-biphenyl]-4-carboxylic acid 40

40 was prepared by Procedure B, 99% yield, (0.28 g, 0.99 mmol, white solid).

¹H NMR (600 MHz, CD₃OD) δ 8.01 (d, J=8.1 Hz, 1H), 7.71-7.66 (m, 2H), 7.58-7.53 (m, 2H), 7.51-7.45 (m, 2H), 7.42-7.36 (m, 1H), 3.13 (t, J=7.7 Hz, 2H), 2.38 (t, J=7.4 Hz, 2H), 2.02-1.94 (m, 2H).

¹³C NMR (151 MHz, CD₃OD) δ 177.6, 171.1, 145.8, 145.4, 141.3, 132.8, 130.7, 130.0, 129.1, 128.16, 125.6, 34.8, 34.7, 28.2.

HRMS (ESI-TOF) Calculated for C₁₇H₁₅O₄ [M−H]⁻: 283.0970, Found: 283.0966.

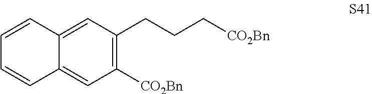 [SMILES: O=C(CCCc1cc2ccccc2cc1C(=O)OCc1ccccc1)OCc1ccccc1]

### Benzyl 3-(4-(benzyloxy)-4-oxobutyl)-2-naphthoate S41

S41 was prepared by Procedure A, 91% yield for two steps, (0.80 g, 1.82 mmol, yellow oil).

¹H NMR (600 MHz, CDCl₃) δ 8.50 (s, 1H), 7.86 (ddq, J=8.2, 1.3, 0.6 Hz, 1H), 7.78-7.73 (m, 1H), 7.64 (d, J=0.8 Hz, 1H), 7.57-7.51 (m, 1H), 7.51-7.44 (m, 3H), 7.43-7.38 (m, 2H), 7.38-7.30 (m, 6H), 5.39 (s, 2H), 5.11 (s, 2H), 3.16-3.11 (m, 2H), 2.38 (t, J=7.5 Hz, 2H), 2.01 (tt, J=9.5, 6.7 Hz, 2H).

¹³C NMR (151 MHz, CDCl₃) δ 173.5, 167.5, 138.9, 136.2, 136.1, 135.1, 132.5, 131.3, 129.6, 128.8, 128.7, 128.6, 128.5, 128.4, 127.9, 127.2, 126.2, 67.0, 66.3, 34.0, 33.9, 26.8. HRMS (ESI-TOF) Calculated for C₂₉H₂₇O₄ [M+H]⁺: 439.1909,

Found: 439.1908.

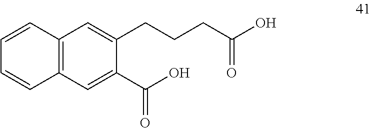 [SMILES: O=C(O)CCCc1cc2ccccc2cc1C(=O)O]

### 3-β-Carboxypropyl)-2-naphthoic acid 41

41 was prepared by Procedure B, 99% yield, (0.26 g, 0.99 mmol, white solid).

¹H NMR (600 MHz, CD₃OD) δ 8.48 (s, 1H), 7.90 (d, J=8.2 Hz, 1H), 7.82 (d, J=8.3 Hz, 1H), 7.72 (s, 1H), 7.55 (ddd, J=8.2, 6.8, 1.3 Hz, 1H), 7.48 (ddd, J=8.1, 6.8, 1.2 Hz, 1H), 3.19-3.14 (m, 2H), 2.36 (t, J=7.5 Hz, 2H), 2.01-1.93 (m, 2H).

¹³C NMR (151 MHz, CD₃OD) δ 177.5, 171.0, 140.1, 136.4, 133.2, 132.7, 130.4, 129.7, 129.6, 129.2, 128.2, 127.2, 34.8, 34.7, 28.1.

HRMS (ESI-TOF) Calculated for C₂₉H₂₇O₄ [M+H]⁺: 439.1909, found: 439.1908.

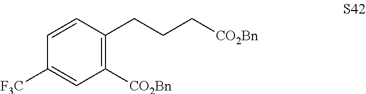 [SMILES: O=C(CCCc1ccc(C(F)(F)F)cc1C(=O)OCc1ccccc1)OCc1ccccc1]

### Benzyl 2-(4-(benzyloxy)-4-oxobutyl)-5-(trifluoromethyl)benzoate S42

S42 was prepared by Procedure A, 88% yield for two steps, (0.80 g, 1.76 mmol, yellow oil).

¹H NMR (600 MHz, CDCl₃) δ 8.18-8.15 (m, 1H), 7.64 (ddd, J=8.1, 2.2, 0.7 Hz, 1H), 7.46-7.30 (m, 11H), 5.35 (s, 2H), 5.11 (s, 2H), 3.05-2.99 (m, 2H), 2.34 (t, J=7.4 Hz, 2H), 1.97-1.89 (m, 2H).

¹³C NMR (151 MHz, CDCl₃) δ 173.1, 166.2, 147.6 (q, J=2.2 Hz), 136.1, 135.6, 131.9, 130.2, 128.9 (q, J=33.0 Hz), 128.7, 128.7, 128.6, 128.6, 128.5, 128.4, 128.4, 128.0 (q, J=4.1 Hz), 123.8 (q, J=272.3 Hz), 67.4, 66.4, 33.9, 33.6, 26.6.

¹⁹F NMR (376 MHz, CDCl₃) δ −65.26.

HRMS (ESI-TOF) Calculated for C₂₆H₂₄F₃O₄ [M+H]⁺: 457.1627, Found: 457.1624.

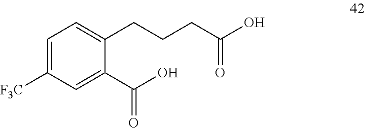 [SMILES: O=C(O)CCCc1ccc(C(F)(F)F)cc1C(=O)O]

### 2-β-Carboxypropyl)-5-(trifluoromethyl)benzoic acid 42

42 was prepared by Procedure B, 99% yield, (0.27 g, 0.99 mmol, white solid).

¹H NMR (600 MHz, CD₃OD) δ 8.15 (d, J=2.0 Hz, 1H), 7.74 (ddd, J=8.0, 2.1, 0.8 Hz, 1H), 7.52 (d, J=8.1 Hz, 1H), 3.13-3.08 (m, 2H), 2.35 (t, J=7.4 Hz, 2H), 1.97-1.89 (m, 2H).

¹³C NMR (151 MHz, CD₃OD) δ 177.1, 169.3, 149.2, 133.2, 129.5 (q, J=33.0 Hz), 129.2 (q, J=3.6 Hz), 128.7 (q, J=3.9 Hz), 125.4 (q, J=271.3 Hz), 34.60, 34.5, 27.8.

¹⁹F NMR (376 MHz, CD₃OD) δ −62.91.

HRMS (ESI-TOF) Calculated for C₁₂H₁₂F₃O₄ [M+H]⁺: 277.0688, Found: 277.0681.

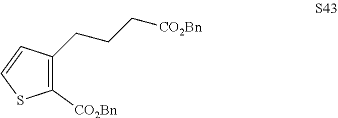 [SMILES: O=C(CCCc1ccsc1C(=O)OCc1ccccc1)OCc1ccccc1]

### Benzyl 3-(4-(benzyloxy)-4-oxobutyl)thiophene-2-carboxylate S43

43 was prepared by Procedure A, 85% yield for two steps, (0.67 g, 1.70 mmol, yellow oil).

¹H NMR (600 MHz, CDCl₃) δ 7.43 (d, J=5.4 Hz, 1H), 7.42-7.27 (m, 10H), 7.03 (d, J=5.4 Hz, 1H), 5.28 (s, 2H), 5.11 (s, 2H), 3.26-3.20 (m, 2H), 2.39 (t, J=8.5 Hz, 2H), 2.07-1.99 (m, 2H).

¹³C NMR (151 MHz, CDCl₃) δ 173.1, 163.2, 154.1, 136.2, 136.1, 129.6, 128.7, 128.7, 128.4, 128.4, 128.4, 128.0, 121.8, 66.3, 33.7, 28.7, 26.7.

HRMS (ESI-TOF) Calculated for C₂₃H₂₃O₄S [M+H]⁺: 395.1317, found: 395.1309.

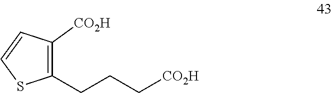 [SMILES: O=C(O)CCCc1sccc1C(=O)O]

### 2-β-carboxypropyl)thiophene-3-carboxylic acid 43

S43 was prepared by Procedure C, 85% yield (182 mg, 0.85 mmol, white solid).

¹H NMR (600 MHz, CD₃OD) δ 7.38 (d, J=5.4 Hz, 1H), 7.18 (dd, J=5.3, 0.8 Hz, 1H), 3.27-3.22 (m, 2H), 2.36 (t, J=7.4 Hz, 2H), 2.02-1.94 (m, 2H).

¹³C NMR (151 MHz, CD₃OD) δ 177.0, 166.5, 155.0, 130.7, 129.8, 129.6, 122.8, 34.2, 29.4, 28.0. HRMS (ESI-TOF) Calculated for C₉H₉O₄S [M−H]⁻: 213.0222, found: 213.0218.

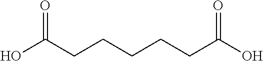 [SMILES: O=C(O)CCCCCC(=O)O]

### Pimelic Acid/Heptane-1,7-Dioic Acid 2

This compound is commercially available.

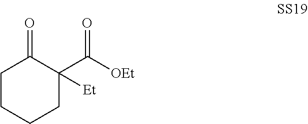 [SMILES: CCOC(=O)C1(CC)CCCCC1=O]

### Ethyl 1-ethyl-2-oxocyclohexane-1-carboxylate SS19

This compound was prepared according to the general procedure B for the preparation of β-substituted heptanedioic acids with the reaction of EtI and methyl 2-oxocyclohexane-1-carboxylate. 75% yield, (1.19 g, 6.0 mmol, colorless oil).

¹H NMR (600 MHz, CDCl₃) δ 4.20 (q, J=7.3 Hz, 2H), 2.53-2.40 (m, 3H), 2.06-1.89 (m, 1H), 1.77-1.56 (m, 2H), 1.44-1.41 (m, 1H), 1.26 (t, J=7.1 Hz, 3H), 0.84 (t, J=7.6 Hz, 3H).

¹³C NMR (151 MHz, CDCl₃) δ 208.4, 172.1, 61.3, 61.2, 41.3, 35.7, 27.8, 27.7, 22.7, 14.3, 8.9.

The data is consistent with those reported in the literature.(33)

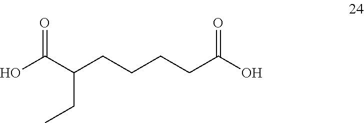

### 2-Ethylheptanedioic acid 24

This compound was prepared according to the general procedure B for the preparation of β-substituted heptanedioic acids. 75% yield, (0.85 g, 4.5 mmol, white solid).

¹H NMR (500 MHz, DMSO) δ 12.00 (s, 2H), 2.18 (t, J=7.3 Hz, 2H), 2.12 (tt, J=8.4, 5.4 Hz, 1H), 1.55-1.40 (m, 5H), 1.40-1.33 (m, 1H), 1.23 (p, J=7.7 Hz, 2H), 0.90-0.74 (m, 3H).

¹³C NMR (126 MHz, DMSO) δ 176.8, 174.4, 46.4, 33.5, 31.2, 26.5, 26.5, 24.8, 24.5, 11.7, 11.6. HRMS (ESI-TOF) Calculated for C₉H₁₅O₄ [M−H]⁻: 187.0970, found: 187.0973.

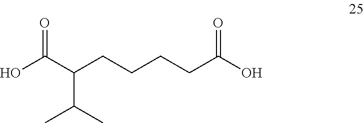

### 2-Isopropylheptanedioic acid 25

This compound was prepared according to the general procedure for the preparation of α-substituted heptanedioic acids.

Elution condition: (20% EA/hexanes+1% AcOH to 50% EA+1% AcOH, gradient elution) Isolated yield: 61% over 2 steps (1.23 g, 6.1 mmol, colourless oil)

¹H NMR (500 MHz, DMSO) δ 11.98 (s, 2H), 2.18 (t, J=7.3 Hz, 2H), 1.95 (ddd, J=9.5, 7.4, 4.8 Hz, 1H), 1.73 (h, J=6.8 Hz, 1H), 1.56-1.44 (m, 2H), 1.42 (ddd, J=9.7, 6.0, 4.0 Hz, 2H), 1.29-1.13 (m, 2H), 0.91-0.82 (m, 6H).

¹³C NMR (126 MHz, DMSO) δ 176.4, 174.4, 174.3, 52.1, 33.6, 30.0, 28.8, 27.0, 24.6, 20.4, 20.0, 19.8.

HRMS (ESI-TOF) Calculated for C₁₀H₁₇O₄ [M−H]⁻: 201.1127, Found: 201.1125.

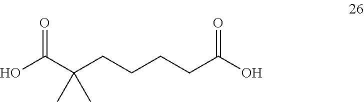

### 2,2-Dimethylheptanedioic acid 26

This compound was prepared according to the general procedure for the preparation of α-substituted heptanedioic acids.

Elution condition: (20% EA/hexanes+1% AcOH to 50% EA+1% AcOH, gradient elution) Isolated yield: 57% over 2 steps (1.07 g, 5.7 mmol, white solid)

¹H NMR (500 MHz, DMSO) δ 3.33 (s, 2H), 2.19 (t, J=7.3 Hz, 2H), 1.54-1.37 (m, 4H), 1.19 (tq, J=12.4, 6.0 Hz, 2H), 1.06 (s, 6H).

¹³C NMR (126 MHz, DMSO) δ 178.8, 174.4, 41.2, 39.9, 33.6, 25.0, 25.0, 24.1. HRMS (ESI-TOF) Calculated for C₉H₁₅O₄ [M−H]⁻: 187.0970,

Found: 187.0972.

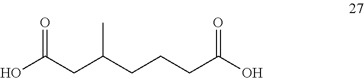

### 3-Methylheptanedioic acid 27

This compound was prepared according to the general procedure for the preparation of β-substituted heptanedioic acids:

Elution condition: (20% EA/hexanes+1% AcOH to 50% EA+1% AcOH, gradient elution) Isolated yield: 20% over 2 steps (0.28 g, 1.6 mmol, pale yellow oil)

¹H NMR (500 MHz, DMSO) δ 11.99 (s, 2H), 2.23-2.12 (m, 3H), 1.99 (dd, J=15.0, 8.0 Hz, 1H), 1.80 (dq, J=13.8, 6.9 Hz, 1H), 1.59-1.39 (m, 2H), 1.28 (ddt, J=13.2, 10.9, 5.6 Hz, 1H), 1.14 (dddd, J=13.2, 10.5, 7.7, 5.4 Hz, 1H), 0.88 (d, J=6.6 Hz, 3H).

¹³C NMR (126 MHz, DMSO) δ 174.4, 173.9, 41.3, 35.5, 33.8, 29.5, 22.0, 19.5. HRMS (ESI-TOF) Calculated for C₈H₁₃O₄ [M−H]⁻: 173.0814,

Found: 173.0815.

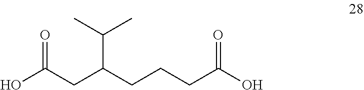

### 3-Isopropylheptanedioic acid 28

This compound was prepared according to the general procedure for the preparation of β-substituted heptanedioic acids:

Elution condition: (20% EA/hexanes+1% AcOH to 50% EA+1% AcOH, gradient elution)

Isolated yield: 50% over 2 steps (0.81 g, 4 mmol, pale yellow oil).

¹H NMR (500 MHz, DMSO) δ 11.97 (s, 2H), 2.25-2.09 (m, 3H), 2.01 (dd, J=15.3, 7.1 Hz, 1H), 1.71-1.60 (m, 2H), 1.53-1.39 (m, 2H), 1.28 (ddt, J=13.2, 11.0, 5.7 Hz, 1H), 1.14 (ddt, J=13.0, 9.9, 6.8 Hz, 1H), 0.80 (dd, J=13.6, 6.5 Hz, 6H).

¹³C NMR (126 MHz, DMSO) δ 174.7, 174.4, 40.0, 35.7, 33.9, 30.2, 29.4, 22.3, 19.2, 18.5. HRMS (ESI-TOF) Calculated for C₁₀H₁₇O₄ [M−H]⁻: 201.1127, Found: 201.1127.

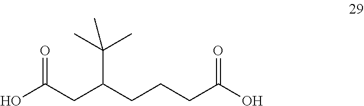

### 3-(tert-Butyl)heptanedioic acid 29

This compound was prepared according to the general procedure for the preparation of β-substituted heptanedioic acids:

Elution condition: (20% EA/hexanes+1% AcOH to 50% EA+1% AcOH, gradient elution)

Isolated yield: 52% over 2 steps (0.9 g, 4.16 mmol, white solid).

¹H NMR (500 MHz, DMSO) δ 11.97 (s, 2H), 2.29 (dd, J=16.2, 5.1 Hz, 1H), 2.25-2.10 (m, 2H), 1.94 (dd, J=16.2, 6.3 Hz, 1H), 1.62-1.51 (m, 2H), 1.51-1.42 (m, 1H), 1.42-1.33 (m, 1H), 0.98 (tdd, J=11.9, 8.1, 3.6 Hz, 1H), 0.82 (s, 9H).

¹³C NMR (126 MHz, DMSO) δ 175.3, 174.4, 44.0, 35.6, 33.9, 33.2, 33.1, 30.2, 27.3, 23.6. HRMS (ESI-TOF) Calculated for C₁₁H₁₉O₄ [M−H]⁻: 215.1283 Found: 215.1284.

### 2.5 Reaction Procedures of the γ-Directed, γ-C—H Lactonization Reaction

General Procedure for the γ-Directed, γ-C—H Lactonization Reaction Using Ag₂CO₃ as Oxidant

To a 2-dram vial was added the substrate (0.1 mmol), Pd(OAc)₂ (10 mol %, 0.01 mmol), ligand (12 mol %, 0.012 mmol), p-xyloquinone (0.2 mmol), Ag₂CO₃ (0.2 mmol), K₂HPO₄ (0.035 mmol) and CsOAc (0.04 mmol, preferably added from a stock solution in HFIP as CsOAc is hygroscopic). HFIP (1.0 mL, or the volume needed to make up to 1.0 mL if a stock solution of CsOAc was used) and a stir-bar was then added, followed by sealing the reaction vessel with a PTFE septum inserted between the vial and its cap. (Note: Pd(OAc)₂, ligand, p-xyloquinone, CsOAc, and the substrate could all be prepared as a stock solution in HFIP. The use of stock solution is recommended for setting up a series of reactions to maximize work efficiency). The reaction mixture was sonicated for 30 seconds before stirring at 200 rpm and 100° C. (heating block temperature) for 36 hours. The reaction mixture was then cooled to room temperature and diluted with dichloromethane (1.0 mL), followed by addition of deionized water (2.0 mL), aq. 6M HCl (0.3 mL), brine (1.0 mL) and then shaken vigorously. The lower organic layer was carefully pipetted and filtered through a short plug of Celite®. The remaining aqueous layer was extracted with CH₂Cl₂ (1.0 mL) twice and the organic layer was pipetted and filtered as mentioned. The combined organic layer was then evaporated to dryness. The crude was then taken into CDCl₃ (0.6 mL) with CH₂Br₂ (10.0 μL) as the internal standard to determine the assay yield of the reaction by ¹H NMR spectroscopy. The isolation of the product was carried out with aqueous extractions of the organic layer (in 0.6 mL CDCl₃ diluted with 2.0 mL CH₂Cl₂) with sat. aq. NaHCO₃ solution (1.0 mL each, 3 times). The collected aqueous layer was then acidified by aq. 6M HCl to pH˜2 and extracted with EtOAc (1.0 mL, 3 times). The combined EtOAc layers was dried with anhydrous MgSO₄, filtered, and evaporated to dryness. The product was either further purified by column chromatography (General procedure for gradient elution unless stated otherwise: begin with 20% EtOAc/hexane+1% AcOH, and increase the composition of EtOAc by 10% for every 8 mL of eluent used to 100% EtOAc+1% AcOH, collecting 1.0-2.0 mL fractions. It was observed that the R_{f} values of carboxylic acids obtained by TLC analysis could not be directly used as a guide for column chromatography. Hence, the gradient elution method was employed for their purification.) or pTLC (exact eluent composition mentioned below for each example) or subject to further derivatization into benzyl esters for isolation if purification of the lactone acid was found to be not straightforward.

### General Procedure for the γ-Directed, γ-C—H Lactonization Reaction Using MnO₂ as Oxidant

To a 2-dram vial was added the substrate (0.1 mmol), Pd(OAc)₂ (10 mol %, 0.01 mmol), ligand (12 mol %, 0.012 mmol), p-xyloquinone (0.2 mmol), MnO₂ (0.4 mmol), K₂HPO₄ (0.021 mmol), KH₂PO₄ (0.032 mmol), CsOAc (0.021 mmol, preferably added from a stock solution in HFIP as CsOAc is hygroscopic). HFIP (1.0 mL, or the volume needed to make up to 1.0 mL if a stock solution of CsOAc was used) and a stir-bar was then added, followed by sealing the reaction vessel with a PTFE septum inserted between the vial and its cap. (Note: Pd(OAc)₂, ligand, p-xyloquinone, CsOAc, and the substrate could all be prepared as a stock solution in HFIP. The use of stock solution is recommended for setting up a series of reactions to maximize work efficiency). The reaction mixture was sonicated for 30 seconds before stirring at 200 rpm and 100° C. (heating block temperature) for 36-48 hours. The reaction mixture was then cooled to room temperature and diluted with dichloromethane (1.0 mL), followed by addition of deionized water (2.0 mL), aq. 6M HCl (0.3 mL), brine (1.0 mL) and then shaken vigorously. The lower organic layer was carefully pipetted and filtered through a short plug of Celite®. The remaining aqueous layer was extracted with CH₂Cl₂ (1.0 mL) twice and the organic layer was pipetted and filtered as mentioned. The combined organic layer was then evaporated to dryness. The crude was then taken into CDCl₃ (0.6 mL) with CH₂Br₂ (10.0 μL) as the internal standard to determine the assay yield of the reaction by ¹H NMR spectroscopy. The isolation of the product was carried out with aqueous extractions of the organic layer (in 0.6 mL CDCl₃ diluted with 2.0 mL CH₂Cl₂) with sat. aq. NaHCO₃ solution (1.0 mL each, 3 times). The collected aqueous layer was then acidified by aq. 6M HCl to pH˜2 and extracted with EtOAc (1.0 mL, 3 times). The combined EtOAc layers was dried with anhydrous MgSO₄, filtered, and evaporated to dryness. The product was either further purified by column chromatography (General procedure for gradient elution unless stated otherwise: begin with 20% EtOAc/hexane+1% AcOH, and increase the composition of EtOAc by 10% for every 8 mL of eluent used to 100% EtOAc+1% AcOH, collecting 1.0-2.0 mL fractions. It was observed that the R_{f} values of carboxylic acids obtained by TLC analysis could not be directly used as a guide for column chromatography. Hence, the gradient elution method was employed for their purification.) or pTLC (exact eluent composition mentioned below for each example) or subject to further derivatization into benzyl esters for isolation if purification of the lactone acid was found to be not straightforward.

### General Procedure for Benzyl Ester Formation

To the product obtained after aqueous extraction with sat. NaHCO₃ solution as mentioned above was added dry CH₂Cl₂ (2.0 mL), BnOH (1.2 eq.), DMAP (1.2 eq.) and EDCI (1.2 eq.) sequentially at room temperature. The reaction mixture was stirred at room temperature overnight, and completion of the reaction was confirmed by TLC analysis of the reaction mixture. The reaction mixture was then quenched by the addition of water and extracted with CH₂Cl₂ (3 times), and the desired product was purified by pTLC (exact eluent composition mentioned below for each example).

2.6 Characterization Data of Products Obtained from γ-Directed, γ-C—H Lactonization Reaction

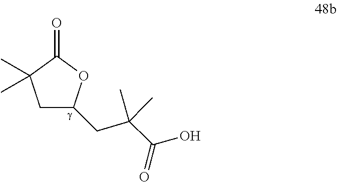

### 3-(4,4-Dimethyl-5-oxotetrahydrofuran-2-yl)-2,2-dimethylpropanoic acid 48b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 100% (21.4 mg, 0.1 mmol, off white solid).

¹H NMR (400 MHz, CDCl₃) δ 4.53 (dq, J=9.9, 5.9 Hz, 1H), 2.19 (dd, J=12.7, 5.9 Hz, 1H), 1.93 (d, J=5.9 Hz, 2H), 1.73 (dd, J=12.7, 9.9 Hz, 1H), 1.31 (s, 3H), 1.29 (s, 3H), 1.25 (s, 3H), 1.24 (s, 3H).

¹³C NMR (100 MHz, CDCl₃) δ 183.8, 182.0, 74.5, 45.9, 44.5, 41.4, 40.1, 26.1, 25.1, 25.1, 24.4. HRMS (ESI-TOF) Calculated for C₁₁H₁₇O₄ [M−H]⁻: 213.1127, Found: 213.1128

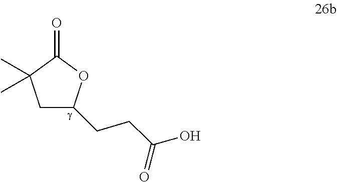

### 3-(4,4-Dimethyl-5-oxotetrahydrofuran-2-yl)propanoic acid 26b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 62% (11.4 mg, 0.062 mmol, off white solid).

¹H NMR (500 MHz, CDCl₃) δ 4.48 (dddd, J=9.9, 8.9, 5.9, 4.1 Hz, 1H), 2.76-2.49 (m, 2H), 2.20 (dd, J=12.7, 5.9 Hz, 1H), 2.02 (dtd, J=15.3, 7.7, 4.1 Hz, 1H), 1.98-1.89 (m, 1H), 1.75 (dd, J=12.7, 9.9 Hz, 1H), 1.28 (s, 3H), 1.26 (s, 3H).

¹³C NMR (126 MHz, CDCl₃) δ 181.8, 178.2, 75.9, 43.4, 40.6, 30.7, 30.1, 25.2, 24.6. HRMS (ESI-TOF) Calculated for C₉H₃O₄ [M−H]⁻: 185.0814, Found: 185.0815.

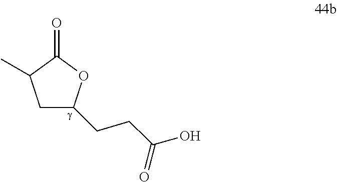

### 3-(4-Methyl-5-oxotetrahydrofuran-2-yl)propanoic acid 44b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 61% (10.5 mg, 0.061 mmol, colorless oil, d.r=1.3:1). Diastereomers were not separable by column chromatography, ¹H and ¹³C NMR data reported as a mixture of diastereomers.

NMR for minor isomer: ¹H NMR (600 MHz, CDCl₃) δ 4.61-4.54 (m, 1H), 2.61-2.48 (m, 3H), 2.18 2.09 (m, 1H), 1.98-1.90 (m, 3H), 1.28 (d, J=7.4 Hz, 3H).

¹³C NMR (151 MHz, CDCl₃) δ 179.9, 178.5, 77.2, 35.5, 34.0, 30.4, 30.1, 15.9.

NMR for major isomer: ¹H NMR (600 MHz, CDCl₃) δ 4.41 (dddd, J=10.1, 8.8, 5.5, 4.1 Hz, 1H), 2.77-2.64 (m, 2H), 2.61-2.48 (m, 2H), 2.08-1.98 (m, 2H), 1.52 (td, J=12.3, 10.4 Hz, 1H), 1.27 (d, J=7.1 Hz, 3H).

¹³C NMR (151 MHz, CDCl₃) δ 179.4, 178.5, 77.2, 37.2, 36.0, 30.5, 30.1, 15.2.

HRMS (ESI-TOF) Calculated for C₈H₁₁O₄ for [M−H]⁻: 171.0657, found 171.0657.

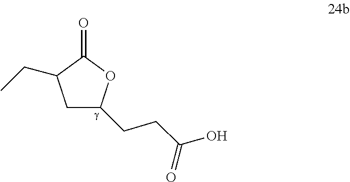

### 3-(4-Ethyl-5-oxotetrahydrofuran-2-yl)propanoic acid 24b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 54% (10.0 mg, 0.054 mmol, colorless oil, d.r.=1.2:1). Diastereomers were not separable by column chromatography, ¹H and ¹³C NMR data reported as a mixture of diastereomers.

NMR for minor isomer: ¹H NMR (600 MHz, CDCl₃) δ 4.59-4.50 (m, 1H), 2.66-2.51 (m, 3H), 2.15-2.06 (m, 2H), 2.07-2.00 (m, 2H), 1.88-1.82 (m, 1H), 1.58-1.42 (m, 1H), 1.01 (t, J=6.6 Hz, 3H).

¹³C NMR (151 MHz, CDCl₃) δ 179.2, 178.3, 77.5, 40.7, 33.1, 30.7, 30.1, 24.1, 11.8.

NMR for major isomer: ¹H NMR (600 MHz, CDCl₃) δ 4.42 (dddd, J=9.9, 8.6, 5.7, 4.1 Hz, 1H), 2.66-2.51 (m, 3H), 1.98-1.89 (m, 4H), 1.58-1.44 (m, 2H), 1.01 (t, J=6.6 Hz, 3H).

¹³C NMR (151 MHz, CDCl₃) δ 178.6, 178.3, 77.5, 42.5, 34.5, 30.6, 30.1, 23.4, 11.7. HRMS (ESI-TOF) Calculated C₉H₁₃₀₄ for [M−H]⁻: 185.0814, found 185.0812.

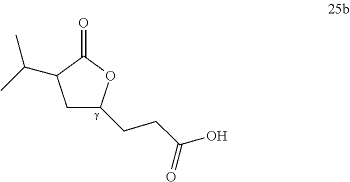

### 3-(4-Isopropyl-5-oxotetrahydrofuran-2-yl)propanoic acid 25b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 74% (14.8 mg, 0.074 mmol, colorless oil, d.r=1.1:1). Diastereomers were not separable by column chromatography, ¹H and ¹³C NMR data reported as a mixture of diastereomers.

NMR for minor diastereomer: ¹H NMR (600 MHz, CDCl₃) δ 4.51 (tt, J=9.0, 4.9 Hz, 1H), 2.64-2.58 (m, 1H), 2.58-2.48 (m, 2H), 2.25-2.21 (m, 1H), 2.16-2.12 (m, 1H), 2.00-1.88 (m, 3H), 1.07-0.98 (m, 3H), 0.91 (dd, J=6.8, 1.1 Hz, 3H).

¹³C NMR (151 MHz, CDCl₃) δ 178.5, 178.2, 77.6, 47.2, 31.1, 30.1, 29.6, 28.9, 20.6, 18.3.

NMR for major diastereomer: ¹H NMR (600 MHz, CDCl₃) δ 4.39 (tdd, J=9.8, 5.6, 4.2 Hz, 1H), 2.64-2.58 (m, 1H), 2.58-2.48 (m, 2H), 2.30 (ddd, J=12.8, 8.8, 5.7 Hz, 1H), 2.21-2.16 (m, 1H), 2.04 (dtd, J=15.2, 7.7, 4.1 Hz, 1H), 2.00-1.88 (m, 1H), 1.64 (td, J=12.4, 10.2 Hz, 1H), 1.07-0.98 (m, 3H), 0.94 (dd, J=6.8, 1.1 Hz, 3H).

¹³C NMR (151 MHz, CDCl₃) δ 178.2, 177.9, 77.1, 45.4, 30.6, 30.3, 30.1, 27.6, 20.8, 18.7. HRMS (ESI-TOF) Calculated for C₁₀H₁₅O₄ [M−H]⁻: 199.0970, Found: 199.0972.

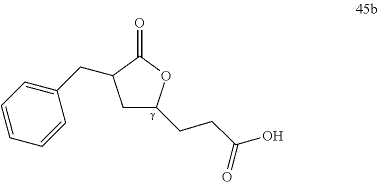 [SMILES: O=C(O)CCC1CC(Cc2ccccc2)C(=O)O1]

### 3-(4-Benzyl-5-oxotetrahydrofuran-2-yl)propanoic acid 45b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 57% (14.1 mg, 0.057 mmol, colorless oil, d.r.=1.2:1). Diastereomers were not separable by column chromatography, ¹H and ¹³C NMR data reported as a mixture of diastereomers.

NMR for minor isomer: ¹H NMR (600 MHz, CDCl₃) δ 7.35-7.13 (m, 5H), 4.44-4.33 (m, 1H), 3.18 (dd, J=13.9, 4.5 Hz, 1H), 3.00-2.89 (m, 1H), 2.78 (dd, J=13.8, 9.4 Hz, 1H), 2.59-2.41 (m, 2H), 2.14 (dt, J=13.2, 7.5 Hz, 1H), 1.91-1.81 (m, 3H).

¹³C NMR (151 MHz, CDCl₃) δ 178.1, 177.9, 138.5, 128.9, 128.9, 126.9, 77.6, 42.9, 36.2, 34.6, 30.4, 29.9.

NMR for major isomer: ¹H NMR (600 MHz, CDCl₃) δ 7.35-7.13 (m, 5H), 4.44-4.33 (m, 1H), 3.28 (dd, J=14.0, 4.3 Hz, 1H), 3.00-2.89 (m, 1H), 2.73 (dd, J=14.1, 9.5 Hz, 1H), 2.59-2.41 (m, 2H), 2.32 (ddd, J=12.7, 8.5, 5.6 Hz, 1H), 1.99-1.91 (m, 2H), 1.58 (td, J=12.3, 10.2 Hz, 1H).

¹³C NMR (151 MHz, CDCl₃) δ 178.1, 177.9, 138.5, 128.9, 128.9, 127.0, 77.6, 42.9, 36.2, 34.6, 30.4, 29.9.

HRMS (ESI-TOF) Calculated for C₁₄H₁₇O₄ [M+H]⁺: 249.1127, Found: 249.1134.

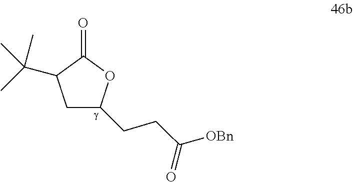 [SMILES: CC(C)(C)C1CC(CCC(=O)OCc2ccccc2)OC1=O]

### Benzyl 3-(4-(tert-butyl)-5-oxotetrahydrofuran-2-yl)propanoate 46b

Following the general procedure for its synthesis and benzyl ester formation, the compound was purified as the benzyl ester by pTLC (10% EA/hexanes, R_{f}=0.30). Isolated yield 57% over 2 steps (17.3 mg, 0.057 mmol, colourless oil, d.r.=1:1). Diastereomers were not separable by column chromatography, ¹H and ¹³C NMR data reported as a mixture of diastereomers.

¹H NMR (600 MHz, CDCl₃) δ 7.41-7.29 (m, 10H), 5.26-4.97 (m, 4H), 4.43 (ddt, J=8.9, 8.0, 4.8 Hz, 1H), 4.29 (dddd, J=10.1, 8.4, 5.6, 4.3 Hz, 1H), 2.62-2.49 (m, 4H), 2.49-2.45 (m, 1H), 2.45-2.41 (m, 1H), 2.35-2.27 (m, 1H), 2.27-2.21 (m, 1H), 2.04 (dddd, J=14.3, 8.3, 7.3, 4.3 Hz, 1H), 1.98-1.88 (m, 4H), 1.66 (td, J=12.6, 10.5 Hz, 1H), 1.04 (s, 18H).

¹³C NMR (151 MHz, CDCl₃) δ 177.2, 176.6, 172.7, 172.7, 135.9, 135.9, 128.7, 128.7, 128.5, 128.5, 128.4, 128.4, 76.7, 76.1, 66.6, 66.6, 50.9, 49.0, 32.9, 31.8, 31.7, 31.3, 30.7, 30.4, 30.4, 30.3, 27.5, 27.3.

HRMS (ESI-TOF) Calculated for C₁₈H₂₅O₄ [M+H]⁺: 305.1753, Found: 305.1754.

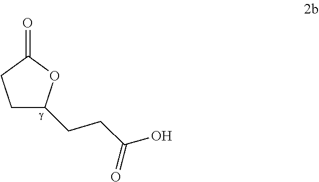 [SMILES: O=C(O)CCC1CCC(=O)O1]

### 3-(5-Oxotetrahydrofuran-2-yl)propanoic acid 2b

Following the general procedure for its synthesis, the compound was purified as the acid by column chromatography (General procedure for gradient elution: begin with 50% EtOAc/hexane+1% AcOH, and increase the composition of EtOAc by 10% for every 8 mL of eluent used to 100% EtOAc+1% AcOH). Isolated yield 62% (9.8 mg, 0.062 mmol, colorless oil).

¹H NMR (600 MHz, CDCl₃) δ 4.57 (dddd, J=9.1, 7.4, 6.8, 4.7 Hz, 1H), 2.74-2.45 (m, 4H), 2.38 (dq, J=12.7, 6.9 Hz, 1H), 2.03 (dq, J=14.6, 7.8 Hz, 1H), 1.96 (dt, J=14.6, 7.4 Hz, 1H), 1.89 (dq, J=12.7, 9.1 Hz, 1H).

¹³C NMR (151 MHz, CDCl₃) δ 176.9, 176.4, 79.6, 30.6, 29.7, 28.8, 28.1. HRMS (ESI-TOF) Calculated for C₇H₉O₄ [M−H]⁻: 157.0501, found: 157.0501.

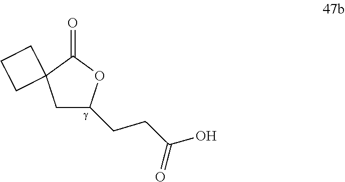 [SMILES: O=C(O)CCC1CC2(CCC2)C(=O)O1]

### 3-(5-Oxo-6-oxaspiro[3.4]octan-7-yl)propanoic acid 47b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 60% (11.9 mg, 0.060 mmol, off white solid).

¹H NMR (500 MHz, CDCl₃) δ 4.39 (tdd, J=8.7, 6.1, 4.1 Hz, 1H), 2.62-2.54 (m, 2H), 2.54-2.47 (m, 2H), 2.47-2.39 (m, 1H), 2.17-2.11 (m, 1H), 2.11-2.05 (m, 1H), 2.02-1.96 (m, 3H), 1.95-1.91 (m, 1H), 1.91-1.85 (m, 1H).

¹³C NMR (126 MHz, CDCl₃) δ 180.9, 178.2, 76.3, 44.5, 41.9, 31.8, 30.6, 30.1, 29.7, 16.6. HRMS (ESI-TOF) Calculated for C₁₀H₁₃O₄ [M−H]⁻: 197.0814, Found: 197.0813.

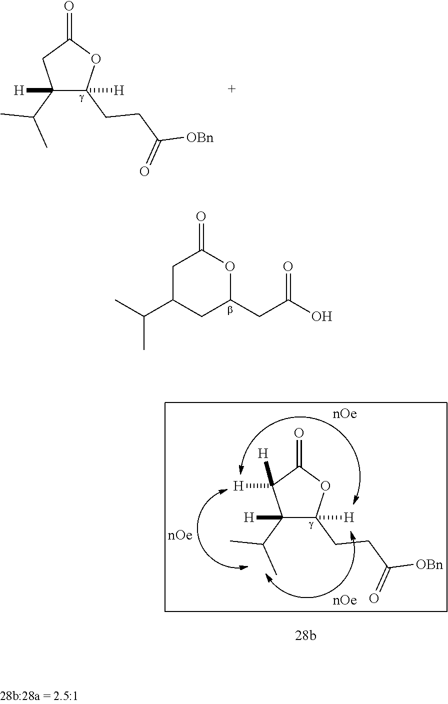

### Benzyl 3-((2S*,3S*)-3-isopropyl-5-oxotetrahydrofuran-2-yl)propanoate 28b

Following the general procedure for its synthesis and benzyl ester formation, the mixture of compound 28b and 28a was obtained as the benzyl ester by pTLC (20% EA/hexanes, R_{f}=0.30). Total isolated yield 50% over 2 steps, (14.5 mg, 0.050 mmol, colourless oil, 28b:28a=2.5:1). A sample of the lactone 28b (7.0 mg, 0.024 mmol, colourless oil) was obtained by further purification (pTLC, 10% EA/hexanes, R_{f}=0.17) for unambiguous characterization.

¹H NMR (500 MHz, CDCl₃) δ 7.54-7.29 (m, 5H), 5.24-5.02 (m, 2H), 4.29 (ddd, J=9.8, 6.5, 3.1 Hz, 1H), 2.76-2.47 (m, 3H), 2.31 (dd, J=18.0, 9.0 Hz, 1H), 2.10 (dtd, J=14.5, 7.9, 3.1 Hz, 1H), 1.98 (dddd, J=9.0, 7.9, 6.5, 6.5 Hz, 1H), 1.85 (dddd, J=14.5, 9.8, 7.5, 5.8 Hz, 1H), 1.71 (h, J=6.8 Hz, 1H), 0.94 (d, J=6.7 Hz, 3H), 0.90 (d, J=6.7 Hz, 3H).

¹³C NMR (126 MHz, CDCl₃) δ 176.5, 172.8, 135.9, 128.7, 128.5, 128.4, 82.8, 66.6, 47.1, 32.3, 31.2, 30.8, 30.5, 20.6, 19.5.

HRMS (ESI-TOF) Calculated for C₁₇H₂₃O₄ [M+H]⁺: 291.1596, Found: 291.1597.

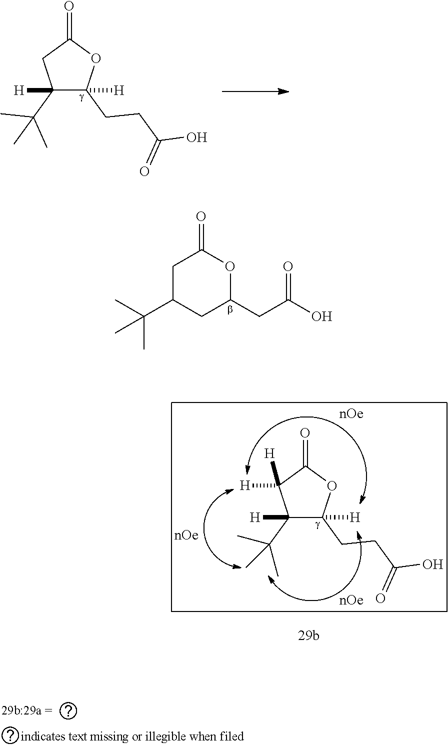

### 3-((2S*,3S*)-3-(tert-butyl)-5-oxotetrahydrofuran-2-yl)propanoic acid 29b

Following the general procedure for its synthesis, the mixture of compounds 29b and 29a was obtained as the acid by column chromatography (Gradient elution: begin with 20% EtOAc/hexane+1% AcOH, and increase the composition of EtOAc by 10% for every 8 mL of eluent used to 70% EtOAc+1% AcOH). Total isolated yield 70% (15.0 mg, 0.070 mmol, colourless oil, 29b:29a=2:1). A sample of the lactone 29b (3.0 mg, 0.014 mmol, white solid) was obtained by further purification (Gradient elution: begin with 20% EtOAc/hexane+1% AcOH, and increase the composition of EtOAc by 10% for every 8 mL of eluent used to 50% EtOAc+1% AcOH, then continue elution using this eluant for the separation of the two lactones) for unambiguous characterization.

¹H NMR (600 MHz, CDCl₃) δ 4.44 (ddd, J=11.3, 4.5, 3.3 Hz, 1H), 2.72-2.52 (m, 3H), 2.42 (dd, J=18.6, 5.5 Hz, 1H), 2.03 (ddd, J=14.5, 7.4, 3.3 Hz, 1H), 1.98 (ddd, J=10.1, 5.5, 4.5 Hz, 1H), 1.87 (ddd, J=14.5, 11.3, 6.5 Hz, 1H), 0.92 (s, 9H).

¹³C NMR (151 MHz, CDCl₃) δ 177.5, 176.9, 81.0, 50.4, 32.4, 32.1, 30.5, 30.0, 26.9. HRMS (ESI-TOF) Calculated for C₁₁H₁₇O₄ [M−H]⁻: 213.1127, Found: 213.1130.

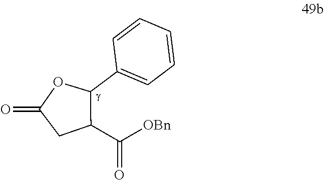 [SMILES: O=C1CC(C(=O)OCc2ccccc2)C(c2ccccc2)O1]

### Benzyl 5-oxo-2-phenyltetrahydrofuran-3-carboxylate 49b

Following the general procedure for its synthesis and benzyl ester formation, the compound was purified as the benzyl ester by pTLC (30% EA/hexanes, R_{f}=0.20). Isolated yield 36% over 2 steps (Total yield 71% for both diastereomers), (10.7 mg, 0.036 mmol, off white solid).

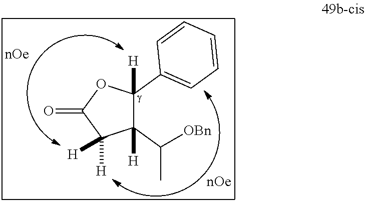

Cis-isomer: ¹H NMR (400 MHz, CDCl₃) δ 7.36-7.28 (m, 6H), 7.25-7.21 (m, 2H), 7.10-7.00 (m, 2H), 5.75 (d, J=7.8 Hz, 1H), 4.79 (d, J=12.1 Hz, 1H), 4.52 (d, J=12.1 Hz, 1H), 3.76 (ddd, J=8.7, 7.8, 5.3 Hz, 1H), 3.11 (dd, J=17.7, 5.3 Hz, 1H), 2.82 (dd, J=17.7, 8.7 Hz, 1H).

¹³C NMR (100 MHz, CDCl₃) δ 175.0, 169.7, 135.2, 134.7, 129.1, 128.7, 128.7, 128.7, 128.7, 125.8, 81.3, 67.4, 46.8, 31.7.

HRMS (ESI-TOF) Calculated for C₁8H₁₇O₄ [M+H]⁺: 297.1127, Found: 297.1118.

Following the general procedure for its synthesis and benzyl ester formation, the compound was purified as the benzyl ester by pTLC (30% EA/hexanes, R_{f}=0.45). Isolated yield 35% over 2 steps (Total yield 71% for both diastereomers), (10.5 mg, 0.035 mmol, off white solid).

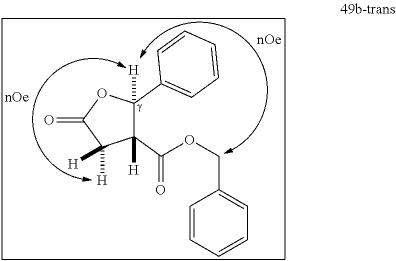

Trans-isomer: ¹H NMR (400 MHz, CDCl₃) δ 7.42-7.33 (m, 6H), 7.33-7.27 (m, 4H), 5.63 (d, J=7.3 Hz, 1H), 5.23 (d, J=12.1 Hz, 1H), 5.17 (d, J=12.2 Hz, 1H), 3.37 (ddd, J=9.4, 8.9, 7.3 Hz, 1H), 3.02 (dd, J=17.7, 8.9 Hz, 1H), 2.91 (dd, J=17.8, 9.4 Hz, 1H).

¹³C NMR (100 MHz, CDCl₃) δ 174.2, 170.7, 138.0, 135.0, 129.1, 129.0, 128.9, 128.9 128.6, 125.6, 82.4, 67.7, 48.9, 32.4.

HRMS (ESI-TOF) Calculated for C₁₈H₁₇O₄ [M+H]⁺: 297.1127, Found: 297.1127.

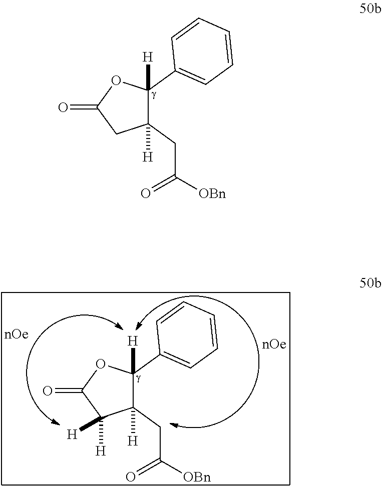

### Benzyl 2-((2S*,3S*)-5-oxo-2-phenyltetrahydrofuran-3-yl)acetate 50b

Following the general procedure for its synthesis and benzyl ester formation, the compound was purified as the benzyl ester by pTLC (20% EA/hexanes, R_{f}=0.25). Isolated yield 25% over 2 steps, (7.8 mg, 0.025 mmol, off white solid).

¹H NMR (500 MHz, CDCl₃) δ 7.42-7.30 (m, 10H), 5.12 (d, J=7.0 Hz, 1H), 5.12-5.05 (m, 2H), 2.91 (dd, J=16.7, 8.2 Hz, 1H), 2.84 (ddddd, J=8.2, 8.2, 8.1, 7.0, 5.6 Hz, 1H), 2.67 (dd, J=16.3, 5.6 Hz, 1H), 2.52 (dd, J=16.3, 8.1 Hz, 1H), 2.42 (dd, J=16.7, 8.2 Hz, 1H).

¹³C NMR (126 MHz, CDCl₃) δ 175.6, 170.8, 137.6, 135.4, 129.1, 129.0, 128.9, 128.8, 128.6, 126.1, 85.5, 67.1, 41.2, 36.4, 34.9.

HRMS (ESI-TOF) Calculated for C₁₉H₁₉O₄ [M+H]⁺: 311.1283, Found: 311.1278.

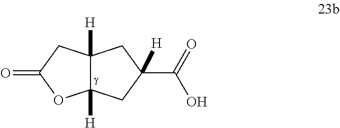 [SMILES: O=C1C[C@@H]2C[C@H](C(=O)O)C[C@@H]2O1]

### (3aS*,5S*,6aS*)-2-Oxohexahydro-2H-cyclopenta[b]furan-5-carboxylic acid 23b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 66% (11.2 mg, 0.066 mmol, off white solid).

¹H NMR (400 MHz, CDCl₃) δ 4.97 (td, J=6.2, 2.4 Hz, 1H), 2.95 (dddd, J=8.8, 8.1, 6.5, 6.3 Hz, 1H), 2.91 (dddd, J=10.0, 8.1, 6.9, 6.2 Hz, 1H), 2.78 (dd, J=17.7, 10.0 Hz, 1H), 2.51 (d, J=17.7 Hz, 1H), 2.42 (ddd, J=15.0, 6.3, 2.4 Hz, 1H), 2.32 (ddd, J=15.1, 8.8, 6.2 Hz, 1H), 2.23 (dt, J=13.4, 8.1 Hz, 1H), 1.94 (dt, J=13.7, 6.9 Hz, 1H).

¹³C NMR (100 MHz, CDCl₃) δ 179.6, 177.3, 85.5, 43.5, 38.8, 36.9, 35.9, 35.1. HRMS (ESI-TOF) Calculated for C₈H₉O₄ [M−H]⁻: 169.0501,

Found: 169.0499.

The relative configuration of 23b was determined from its reduced analogue 23b′ through treatment with BH₃·THF.

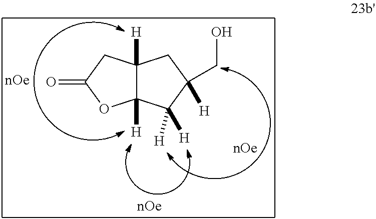

Compound 23b (50.0 mg, 0.30 mmol) was dissolved in anhydrous THF (2.0 mL), the reaction mixture was cooled to 0° C. and a solution of BH₃·THF (0.9 mL, 0.9 mmol, 1.0 M in THF) was added. The reaction was gradually warmed to room temperature and stirred overnight. TLC analysis at this stage suggested completion of reaction. The reaction was quenched by addition of MeOH and all the volatiles were removed under reduced pressure. The desired compound was then purified by flash column chromatography (100% EA, R_{f}=0.4) and obtained as a colourless liquid (45.0 mg, 0.29 mmol, 97%).

¹H NMR (500 MHz, CDCl₃) δ 4.93 (td, J=6.8, 3.5 Hz, 1H), 3.68-3.48 (m, 2H), 2.84-2.77 (m, 1H), 2.73 (dd, J=17.6, 9.1 Hz, 1H), 2.36 (dd, J=17.6, 1.5 Hz, 1H), 2.32-2.21 (m, 1H), 2.22-2.12 (m, 3H), 1.67 (ddd, J=14.1, 8.6, 3.5 Hz, 1H), 1.27-1.14 (m, 1H).

¹³C NMR (126 MHz, CDCl₃) δ 177.5, 86.2, 65.8, 42.3, 39.4, 35.8, 35.7, 35.7.

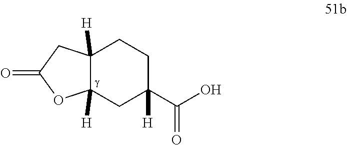 [SMILES: O=C1C[C@@H]2CC[C@H](C(=O)O)C[C@@H]2O1]

### (3aS*,6S*,7aS*)-2-Oxooctahydrobenzofuran-6-carboxylic acid 51b

Following the general procedure for its synthesis, the compound was purified as the acid by column chromatography (General procedure for gradient elution: begin with 20% EtOAc/hexane+1% AcOH, and increase the composition of EtOAc by 10% for every 8 mL of eluent used to 100% EtOAc+1% AcOH). Isolated yield 60% based on reactive diastereomers* (7.6 mg, 0.042 mmol, off white solid).

¹H NMR (500 MHz, CDCl₃) δ 4.57 (dt, J=8.9, 6.0 Hz, 1H), 2.75-2.59 (m, 1H), 2.47 (dd, J=17.2, 7.8 Hz, 1H), 2.44-2.37 (m, 2H), 2.29 (dddd, J=13.9, 5.5, 4.0, 1.4 Hz, 1H), 1.90-1.85 (m, 1H), 1.85-1.78 (m, 2H), 1.75-1.66 (m, 2H).

¹³C NMR (126 MHz, CDCl₃) δ 179.3, 176.6, 78.0, 38.3, 34.0, 32.8, 30.2, 24.1, 22.9. HRMS (ESI-TOF) Calculated for C₉H₁O₄ [M−H]⁻: 183.0657, Found: 183.0660.

*It was observed that only the syn-diacid of 51 provided the lactone 51b, whereas the anti-diacid of 51 was not reactive.

The relative configuration of 51b was determined from its benzyl ester analogue 51b′, the synthesis of benzyl ester 51b′ was carried out according to the general procedure as described above.

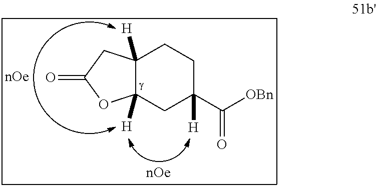 [SMILES: O=C1C[C@@H]2CC[C@H](C(=O)OCc3ccccc3)C[C@@H]2O1]

¹H NMR (500 MHz, CDCl₃) δ 7.40-7.30 (m, 5H), 5.13 (s, 2H), 4.55 (ddd, J=9.3, 6.2, 5.0 Hz, 1H), 2.70-2.62 (m, 1H), 2.46-2.39 (m, 2H), 2.41-2.36 (m, 1H), 2.30 (dt, J=13.8, 5.0 Hz, 1H), 1.91-1.84 (m, 1H), 1.84-1.79 (m, 1H), 1.79-1.74 (m, 1H), 1.74-1.63 (m, 2H).

¹³C NMR (126 MHz, CDCl₃) δ 176.5, 173.8, 135.9, 128.8, 128.5, 128.4, 78.1, 66.8, 38.8, 34.1, 32.5, 30.6, 24.2, 23.1.

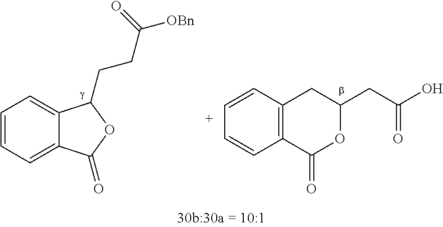 [SMILES: O=C(CCC1OC(=O)c2ccccc21)OCc1ccccc1 ; O=C(O)CC1Cc2ccccc2C(=O)O1]

### Benzyl 3-β-oxo-1,3-dihydroisobenzofuran-1-yl)propanoate 30b

Following the general procedure for its synthesis and benzyl ester formation, the compound was purified as the benzyl ester by pTLC (20% EA/hexanes, R_{f}=0.30). Isolated yield 57% over 2 steps, (16.9 mg, 0.057 mmol, off white solid, 30b:30a=10:1 by ¹H NMR).

¹H NMR (500 MHz, CDCl₃) δ 7.90 (d, J=7.5 Hz, 1H), 7.68 (td, J=7.5, 1.0 Hz, 1H), 7.54 (t, J=7.5 Hz, 1H), 7.46 (dd, J=7.5, 1.0 Hz, 1H), 7.39-7.30 (m, 5H), 5.55 (dd, J=8.7, 2.8 Hz, 1H), 5.12 (s, 2H), 2.70-2.57 (m, 1H), 2.57-2.44 (m, 2H), 2.08-1.91 (m, 1H).

¹³C NMR (126 MHz, CDCl₃) δ 172.6, 170.3, 149.3, 135.8, 134.3, 129.5, 128.8, 128.5, 128.4, 126.2, 126.0, 122.0, 80.1, 66.7, 30.0, 29.6.

HRMS (ESI-TOF) Calculated for C₁₈H₁₇O₄ [M+H]⁺: 297.1127, Found: 297.1129.

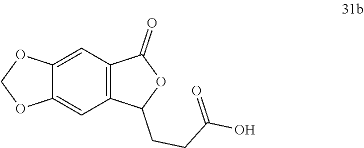 [SMILES: O=C(O)CCC1OC(=O)c2cc3c(cc21)OCO3]

### 3-(7-Oxo-5,7-dihydro-[1,3]dioxolo[4,5-f]isobenzofuran-5-yl)propanoic acid 31b

Following the general procedure for its synthesis, the compound was purified by pTLC (50% EA/hexanes, 2% AcOH). Isolated yield 75% (18.7 mg, 0.075 mmol, off white solid).

¹H NMR (600 MHz, DMSO-d6) δ 7.24-7.21 (m, 2H), 6.21 (dd, J=3.5, 1.0 Hz, 2H), 5.49 (dd, J=7.5, 3.9 Hz, 1H), 2.37-2.27 (m, 2H), 2.27-2.18 (m, 1H), 1.89-1.80 (m, 1H).

¹³C NMR (151 MHz, DMSO-d6) δ 173.6, 169.2, 153.5, 149.1, 146.4, 118.7, 103.3, 102.9, 102.4, 79.4, 29.3, 29.1.

HRMS (ESI-TOF) Calculated for C₁₂H₁₀O₆Na [M+Na]⁺: 273.0375, Found: 273.0384.

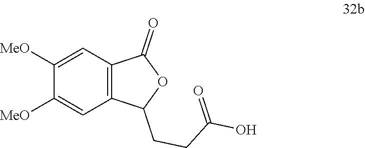 [SMILES: COc1cc2c(cc1OC)C(CCC(=O)O)OC2=O]

### 3-(5,6-Dimethoxy-3-oxo-1,3-dihydroisobenzofuran-1-yl)propanoic acid 32b

Following the general procedure for its synthesis, the compound was purified by pTLC (50% EA/hexanes, 2% AcOH). Isolated yield 61% (16.2 mg, 0.061 mmol, off white solid).

¹H NMR (600 MHz, CD₃OD) δ 7.29 (s, 1H), 7.14 (d, J=0.8 Hz, 1H), 5.54-5.49 (m, 1H), 3.96 (s, 3H), 3.90 (s, 3H), 2.53-2.35 (m, 3H), 2.03-1.94 (m, 1H).

¹³C NMR (151 MHz, CD₃OD) δ 174.9, 172.8, 156.9, 152.3, 145.8, 118.6, 106.9, 105.2, 81.5, 56.9, 56.7, 30.8, 30.0.

HRMS (ESI-TOF) Calculated for C₁₃H₁₅O₆ [M+H]⁺: 267.0869, Found: 267.0865.

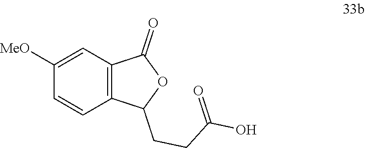 [SMILES: COc1ccc2c(c1)C(=O)OC2CCC(=O)O]

### 3-(5-Methoxy-3-oxo-1,3-dihydroisobenzofuran-1-yl)propanoic acid 33b

Following the general procedure for its synthesis, the compound was purified by pTLC (50% EA/hexanes, 2% AcOH). Isolated yield 70% (16.5 mg, 0.07 mmol, off white solid).

¹H NMR (600 MHz, CD₃OD) δ 7.53-7.48 (m, 1H), 7.35-7.30 (m, 2H), 5.57 (dd, J=8.0, 3.3 Hz, 1H), 3.88 (s, 3H), 2.50-2.34 (m, 3H), 2.00-1.90 (m, 1H).

¹³C NMR (151 MHz, CD₃OD) δ 176.9, 172.4, 162.4, 143.5, 128.4, 124.4, 124.0, 108.5, 82.1, 56.3, 31.3, 30.6.

HRMS (ESI-TOF) Calculated for C₁₂H₁₃O₅ [M+H]⁺: 237.0763, Found: 237.0762.

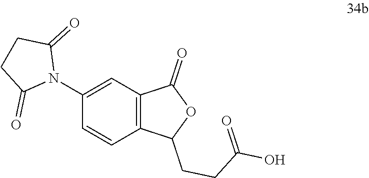 [SMILES: O=C(O)CCC1OC(=O)c2cc(N3C(=O)CCC3=O)ccc21]

### 3-(6-(2,5-Dioxopyrrolidin-1-yl)-3-oxo-1,3-dihydroisobenzofuran-1-yl)propanoic acid 34b

Following the general procedure for its synthesis, the compound was purified by pTLC (70% EA/hexanes, 2% AcOH). Isolated yield 50% (15.1 mg, 0.050 mmol, off white solid, 34b:34a=5:1 by ¹H NMR).

¹H NMR (600 MHz, CD₃OD) δ 7.83 (dd, J=1.8, 0.7 Hz, 1H), 7.76-7.68 (m, 2H), 5.69 (dd, J=8.4, 3.0 Hz, 1H), 2.89 (s, 4H), 2.56-2.39 (m, 3H), 2.02-1.98 (m, 1H).

¹³C NMR (151 MHz, CD₃OD) δ 178.6, 176.2, 171.3, 150.7, 135.2, 134.2, 127.9, 124.9, 124.2, 82.2, 30.9, 30.3, 29.5.

HRMS (ESI-TOF) Calculated for C₁₅H₁₄NO₆ [M+H]⁺: 304.0821, Found: 304.0824.

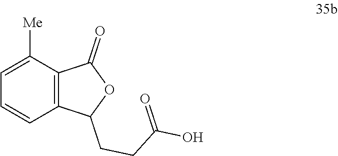 [SMILES: Cc1cccc2c1C(=O)OC2CCC(=O)O]

### 3-(4-Methyl-3-oxo-1,3-dihydroisobenzofuran-1-yl)propanoic acid 35b

Following the general procedure for its synthesis, the compound was purified by pTLC (50% EA/hexanes, 2% AcOH). Isolated yield 73% (16.0 mg, 0.073 mmol, off white solid, 35b:35a=8:1 by ¹H NMR).

¹H NMR (600 MHz, CDCl₃) δ 7.53 (t, J=7.6 Hz, 1H), 7.30-7.24 (m, 2H), 5.48 (dd, J=8.5, 3.2 Hz, 1H), 2.68 (s, 3H), 2.67-2.58 (m, 1H), 2.54-2.41 (m, 2H), 2.00-1.92 (m, 1H).

¹³C NMR (151 MHz, CDCl₃) δ 178.1, 170.6, 149.7, 140.1, 134.1, 131.2, 123.6, 119.2, 79.0, 29.9, 29.3, 17.5.

HRMS (ESI-TOF) Calculated for C₁₂H₁₃O₄ [M+H]⁺: 221.0814, Found: 221.0814.

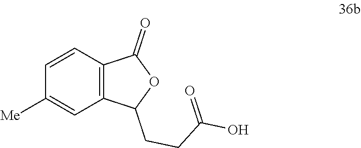 [SMILES: Cc1ccc2c(c1)C(CCC(=O)O)OC2=O]

### 3-(6-Methyl-3-oxo-1,3-dihydroisobenzofuran-1-yl)propanoic acid 36b

Following the general procedure for its synthesis, the compound was purified by pTLC (50% EA/hexanes, 2% AcOH). Isolated yield 55% (12.1 mg, 0.055 mmol, off white solid, 36b:36a=9:1 by ¹H NMR).

¹H NMR (600 MHz, CDCl₃) δ 7.77 (d, J=7.8 Hz, 1H), 7.33 (d, J=7.8 Hz, 1H), 7.25 (s, 2H), 5.50 (dd, J=8.6, 3.1 Hz, 1H), 2.64-2.57 (m, 1H), 2.53-2.41 (m, 5H), 2.01-1.92 (m, 1H).

¹³C NMR (151 MHz, CDCl₃) δ 177.4, 170.5, 149.8, 145.7, 130.7, 125.8, 123.6, 122.3, 79.8, 29.9, 29.4, 22.2.

HRMS (ESI-TOF) Calculated for C₁₂H₁₃O₄ [M+H]⁺: 221.0814, Found: 221.0813.

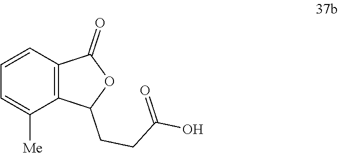 [SMILES: Cc1cccc2c1C(CCC(=O)O)OC2=O]

### 3-(7-Methyl-3-oxo-1,3-dihydroisobenzofuran-1-yl)propanoic acid 37b

Following the general procedure for its synthesis, the compound was purified by pTLC (50% EA/hexanes, 2% AcOH). Isolated yield 60% (13.2 mg, 0.055 mmol, off white solid, 37b:37a=1.6:1 by 1H NMR).

¹H NMR (600 MHz, CDCl₃) δ 7.73 (dd, J=6.5, 2.3 Hz, 1H), 7.47-7.41 (m, 2H), 5.59 (dd, J=8.6, 2.5 Hz, 1H), 2.67-2.57 (m, 2H), 2.51-2.44 (m, 1H), 2.43 (s, 3H), 1.96-1.87 (m, 1H).

¹³C NMR (151 MHz, CDCl₃) δ 177.5, 170.7, 147.5, 135.8, 132.7, 129.8, 126.1, 123.5, 79.9, 29.3, 28.4, 18.2.

HRMS (ESI-TOF) Calculated for C₁₂H₁₃O₄ [M+H]⁺: 221.0814, Found: 221.0816.

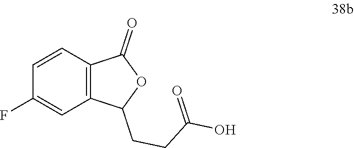 [SMILES: O=C(O)CCC1OC(=O)c2ccc(F)cc21]

### 3-(6-Fluoro-3-oxo-1,3-dihydroisobenzofuran-1-yl)propanoic acid 38b

Following the general procedure for its synthesis, the compound was purified by pTLC (50% EA/hexanes, 2% AcOH). Isolated yield 68% (15.2 mg, 0.068 mmol, off white solid, 38b:38a=10:1 by ¹H NMR).

¹H NMR (600 MHz, CDCl₃) δ 7.90 (dd, J=8.5, 4.8 Hz, 1H), 7.24 (td, J=8.5, 2.1 Hz, 1H), 7.16 (dd, J=7.6, 2.4 Hz, 1H), 5.54 (dd, J=8.6, 3.5 Hz, 1H), 2.69-2.61 (m, 1H), 2.55-2.50 (m, 1H), 2.50-2.42 (m, 1H), 2.03-1.94 (m, 1H).

¹³C NMR (151 MHz, CDCl₃) δ 177.5, 169.1, 167.6, 165.9, 152.0 (d, J=10.4 Hz), 128.5 (d, J=10.1 Hz), 117.9 (d, J=23.8 Hz), 109.4 (d, J=24.3 Hz), 79.3 (d, J=2.8 Hz), 29.7, 29.2.

¹⁹F NMR (471 MHz, CDCl₃) δ −105.19.

HRMS (ESI-TOF) Calculated for C₁₁H₁₀FO₄ [M+H]⁺: 225.0563, Found: 225.0559.

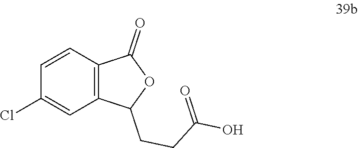 [SMILES: O=C(O)CCC1OC(=O)c2ccc(Cl)cc21]

### 3-(6-Chloro-3-oxo-1,3-dihydroisobenzofuran-1-yl)propanoic acid 39b

Following the general procedure for its synthesis, the compound was purified by pTLC (50% EA/hexanes, 2% AcOH). Isolated yield 42% (10.1 mg, 0.042 mmol, off white solid, 39b:39a=5:1 by ¹H NMR).

¹H NMR (600 MHz, CDCl₃) δ 7.84 (d, J=8.1 Hz, 1H), 7.53 (dd, J=8.2, 1.7 Hz, 1H), 7.49-7.47 (m, 1H), 5.54 (dd, J=8.8, 3.4 Hz, 1H), 2.70-2.61 (m, 1H), 2.58-2.43 (m, 2H), 2.03-1.94 (m, 1H).

¹³C NMR (151 MHz, CDCl₃) δ 174.8, 169.2, 150.8, 141.2, 130.4, 127.3, 124.7, 122.5, 79.4, 29.7, 29.2. HRMS (ESI-TOF) Calculated for C₁₁H₁₀ClO₄ [M+H]⁺: 241.0268, Found: 241.0268.

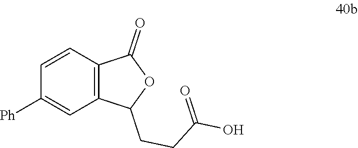 [SMILES: O=C(O)CCC1OC(=O)c2ccc(-c3ccccc3)cc21]

### 3-β-Oxo-6-phenyl-1,3-dihydroisobenzofuran-1-yl)propanoic acid 40b

Following the general procedure for its synthesis, the compound was purified by pTLC (50% EA/hexanes, 2% AcOH). Isolated yield 50% (14.1 mg, 0.05 mmol, off white solid, 40b:40a=16:1 by ¹H NMR).

¹H NMR (600 MHz, CDCl₃) δ 7.95 (d, J=7.9 Hz, 1H), 7.75 (dd, J=8.0, 1.4 Hz, 1H), 7.65-7.59 (m, 3H), 7.52-7.49 (m, 2H), 7.49-7.41 (m, 2H), 5.60 (dd, J=8.0, 2.1 Hz, 1H), 2.69-2.61 (m, 1H), 2.58-2.50 (m, 2H), 2.09-1.99 (m, 1H).

¹³C NMR (151 MHz, CDCl₃) δ 177.1, 170.3, 150.1, 147.9, 139.7, 129.3, 128.9, 128.9, 127.69, 126.4, 124.9, 120.5, 80.0, 29.9, 29.3.

HRMS (ESI-TOF) Calculated for C₇₁H₁₅O₄ [M+H]⁺: 283.0970, Found: 283.0964.

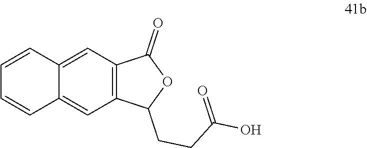 [SMILES: O=C(O)CCC1OC(=O)c2cc3ccccc3cc21]

### 3-β-Oxo-1,3-dihydronaphtho[2,3-c]furan-1-yl)propanoic acid 41b

Following the general procedure for its synthesis, the compound was purified by pTLC (50% EA/hexanes, 2% AcOH). Isolated yield 60% (15.4 mg, 0.06 mmol, off white solid, 41b:41a=10:1 by ¹H NMR).

¹H NMR (600 MHz, CD₃OD) δ 8.43 (s, 1H), 8.08 (dd, J=8.3, 1.2 Hz, 1H), 8.07-8.00 (m, 2H), 7.67 (ddd, J=8.2, 6.8, 1.3 Hz, 1H), 7.60 (ddd, J=8.2, 6.8, 1.2 Hz, 1H), 5.76 (dd, J=8.4, 3.4 Hz, 1H), 2.60-2.48 (m, 2H), 2.48-2.37 (m, 1H), 2.13-1.96 (m, 1H).

¹³C NMR (151 MHz, CD₃OD) δ 176.6, 172.2, 144.6, 137.9, 134.7, 130.9, 130.2, 129.5, 128.2, 127.7, 124.8, 122.3, 82.2, 31.7, 30.5.

HRMS (ESI-TOF) Calculated for C₁₅H₁₃O₄ [M+H]⁺: 257.0814, Found: 257.0814.

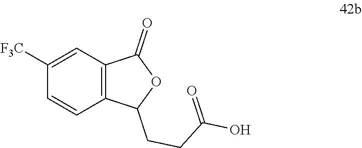 [SMILES: O=C(O)CCC1OC(=O)c2cc(C(F)(F)F)ccc21]

### 3-β-Oxo-5-(trifluoromethyl)-1,3-dihydroisobenzofuran-1-yl)propanoic acid 42b

Following the general procedure for its synthesis, the compound was purified by pTLC (50% EA/hexanes, 2% AcOH). Isolated yield 40% (10.1 mg, 0.04 mmol, off white solid).

¹H NMR (600 MHz, CD₃OD) δ 8.15 (s, 1H), 8.07 (dd, J=8.4, 1.5 Hz, 1H), 7.89-7.84 (m, 1H), 5.75 (dd, J=8.4, 3.1 Hz, 1H), 2.57-2.48 (m, 2H), 2.48-2.40 (m, 1H), 2.07-1.97 (m, 1H).

¹³C NMR (151 MHz, CD₃OD) δ 176.4, 170.6, 154.7, 133.1 (q, J=33.0 Hz), 132.3 (q, J=3.3 Hz), 128.2, 125.1 (d, J=271.8 Hz), 123.5 (q, J=4.1 Hz), 125.0, 82.3, 30.8, 30.5.

¹⁹F NMR (376 MHz, CD₃OD) δ −66.59.

HRMS (ESI-TOF) Calculated for C₁₂H₁₀F₃O₄ [M+H]⁺: 275.0531, Found: 275.0531.

### 2.4 Synthesis and Characterization of Substrates for the γ-Directed, γ-C—H Lactonization Reaction

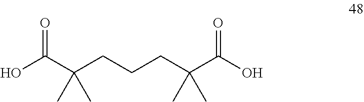

### 2,2,6,6-Tetramethylheptanedioic acid 48

This compound is commercially available.

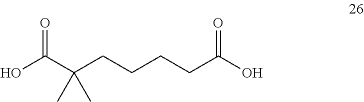

### 2,2-Dimethylheptanedioic acid 26

Please see Section 2.4

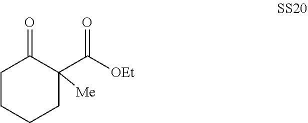 [SMILES: CCOC(=O)C1(C)CCCCC1=O]

### Ethyl 1-methyl-2-oxocyclohexane-1-carboxylate SS20

This compound was prepared according to the general procedure B for the preparation of β-substituted heptanedioic acids with the reaction of MeI and ethyl 2-oxocyclohexane-1-carboxylate. 73% yield, (1.08 g, 5.86 mmol, colorless oil).

¹H NMR (600 MHz, CDCl₃) δ 4.22-4.14 (m, 2H), 2.54-2.41 (m, 3H), 2.06-1.97 (m, 1H), 1.78-1.57 (m, 4H), 1.50-1.41 (m, 1H), 1.28 (s, 3H), 1.25 (t, J=7.1 Hz, 3H).

¹³C NMR (151 MHz, CDCl₃) δ 208.5, 173.2, 61.4, 57.3, 40.8, 38.4, 27.7, 22.8, 21.4, 14.2.

The data is consistent with those reported in the literature.(33)

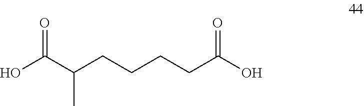

### 2-Methylheptanedioic acid 44

This compound was prepared according to the general procedure B for the preparation of β-substituted heptanedioic acids. 75% yield, (0.78 g, 4.5 mmol, white solid).

¹H NMR (500 MHz, DMSO) δ 2.29 (h, J=6.9 Hz, 1H), 2.19 (t, J=7.3 Hz, 2H), 1.57-1.50 (m, 1H), 1.50-1.41 (m, 2H), 1.33 (dq, J=15.7, 6.8 Hz, 1H), 1.25 (qd, J=7.6, 3.7 Hz, 2H), 1.03 (d, J=6.9 Hz, 3H).

¹³C NMR (126 MHz, DMSO) δ 177.4, 174.4, 38.6, 33.6, 33.0, 26.3, 24.4, 17.0. HRMS (ESI-TOF) Calculated for C₈H₁₃O₄, [M−H]⁻: 173.0814, found: 173.0811.

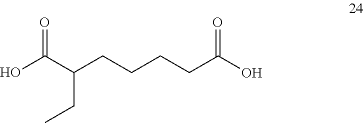

### 2-Ethylheptanedioic acid 24

Please see Section 2.4

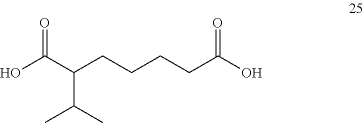

### 2-Isopropyiheptanedioic acid 25

Please see Section 2.4

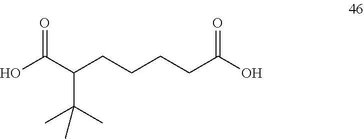

### 2-(tert-Butyl)heptanedioic acid 46

This compound was prepared according to the general procedure for the preparation of α-substituted heptanedioic acids.

Elution condition: (20% EA/hexanes+1% AcOH to 50% EA+1% AcOH, gradient elution) Isolated yield: 44% over 2 steps (0.95 g, 4.4 mmol, colourless oil)

¹H NMR (500 MHz, DMSO) δ 11.97 (s, 2H), 2.18 (t, J=7.4 Hz, 2H), 1.97 (dd, J=11.8, 2.9 Hz, 1H), 1.55-1.43 (m, 3H), 1.39 (dddd, J=12.8, 9.4, 6.3, 2.8 Hz, 1H), 1.26-1.11 (m, 2H), 0.91 (s, 9H).

¹³C NMR (126 MHz, DMSO) δ 176.1, 174.4, 55.6, 33.6, 32.1, 27.7, 27.6, 26.9, 24.5. HRMS (ESI-TOF) Calculated for C₁₁H₁₉O₄ [M−H]⁻: 215.1283, Found: 215.1281.

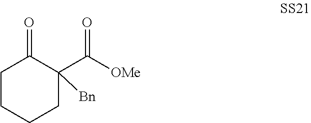 [SMILES: COC(=O)C1(Cc2ccccc2)CCCCC1=O]

### Methyl 1-benzyl-2-oxocyclohexane-1-carboxylate SS21

This compound was prepared according to the general procedure B for the preparation of α-substituted heptanedioic acids with the reaction of BnBr and methyl 2-oxocyclohexane-1-carboxylate. 95% yield, (1.87 g, 7.6 mmol, white solid).

¹H NMR (600 MHz, CDCl₃) δ 7.27-7.22 (m, 2H), 7.22-7.17 (m, 1H), 7.11-7.07 (m, 2H), 3.64 (s, 3H), 3.32 (d, J=13.8 Hz, 1H), 2.87 (d, J=13.8 Hz, 1H), 2.52-2.35 (m, 3H), 2.05-1.97 (m, 1H), 1.78-1.57 (m, 3H), 1.46 (ddd, J=13.8, 12.1, 4.5 Hz, 1H).

¹³C NMR (151 MHz, CDCl₃) δ 207.3, 171.6, 136.7, 130.4, 128.2, 126.8, 62.4, 52.3, 41.5, 40.6, 36.0, 27.7, 22.6.

The data is consistent with those reported in the literature.(37)

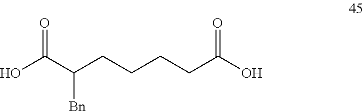 [SMILES: O=C(O)CCCCC(Cc1ccccc1)C(=O)O]

### 2-Benzylheptanedioic acid 45

This compound was prepared according to the general procedure B for the preparation of α-substituted heptanedioic acids. 85% yield, (1.28 g, 5.1 mmol, white solid).

¹H NMR (600 MHz, CDCl₃) δ 7.32-7.27 (m, 2H), 7.24-7.20 (m, 1H), 7.19-7.15 (m, 2H), 3.06-2.98 (m, 1H), 2.74-2.65 (m, 2H), 2.40 (ddd, J=15.2, 8.1, 4.4 Hz, 1H), 2.32 (ddd, J=15.3, 8.6, 4.4 Hz, 1H), 1.72-1.48 (m, 4H), 1.44-1.25 (m, 2H).

¹³C NMR (151 MHz, CDCl₃) δ 182.5, 180.7, 139.0, 129.0, 128.6, 126.6, 47.6, 38.4, 33.9, 31.5, 26.2, 24.7.

HRMS (ESI-TOF) Calculated for C₁₄H₁₈O₄Na [M+Na]⁺: 273.1103, Found: 273.1095.

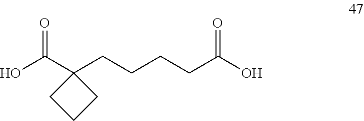 [SMILES: O=C(O)CCCCC1(C(=O)O)CCC1]

### 1-(4-Carboxybutyl)cyclobutane-1-carboxylic acid 47

This compound was prepared according to the general procedure for the preparation of α-substituted heptanedioic acids.

Elution condition: (20% EA/hexanes+1% AcOH to 50% EA/hexanes+1% AcOH, gradient elution)

Isolated yield: 30% over 2 steps (0.60 g, 3.0 mmol, off-white solid)

¹H NMR (500 MHz, DMSO) δ 12.01 (s, 2H), 2.27 (qd, J=8.0, 4.4 Hz, 2H), 2.19 (t, J=7.3 Hz, 2H), 1.89-1.71 (m, 4H), 1.71-1.63 (m, 2H), 1.47 (p, J=7.5 Hz, 2H), 1.20-1.07 (m, 2H).

¹³C NMR (126 MHz, DMSO) δ 177.9, 174.4, 46.9, 37.3, 33.6, 33.5, 29.5, 24.8, 24.1, 15.0. HRMS (ESI-TOF) Calculated for C₁₀H₁₅O₄ [M−H]⁻: 199.0970, Found: 199.0971.

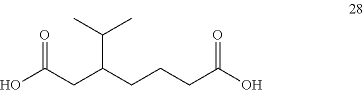

### 3-Isopropyiheptanedioic acid 28

Please see Section 2.4

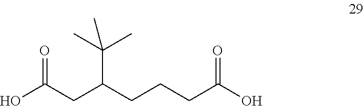

### 3-(tert-Butyl)heptanedioic acid 29

Please see Section 2.4

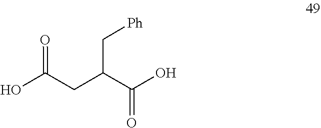 [SMILES: O=C(O)CC(Cc1ccccc1)C(=O)O]

### 2-Benzylsuccinic acid 49

This compound is commercially available.

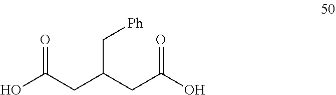 [SMILES: O=C(O)CC(CC(=O)O)Cc1ccccc1]

### 3-Benzylpentanedioic acid 50

This compound was prepared exactly according to the procedures published by Bonati and co-workers.(38)

¹H NMR (500 MHz, DMSO) δ 12.15 (s, 2H), 7.36-7.26 (m, 2H), 7.24-7.18 (m, 1H), 7.18-7.12 (m, 2H), 2.60 (d, J=6.9 Hz, 2H), 2.38 (p, J=6.7 Hz, 1H), 2.26-2.14 (m, 4H). ¹³C NMR (126 MHz, DMSO) δ 173.5, 139.6, 129.1, 128.3, 126.1, 39.0, 37.2, 33.5. HRMS (ESI-TOF) Calculated for C₁₂H₁₃O₄ [M−H]⁻: 221.0814, Found: 221.0815.

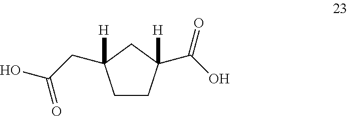 [SMILES: O=C(O)C[C@H]1CC[C@@H](C(=O)O)C1]

### (1R*,3S*)-3-(Carboxymethyl)cyclopentane-1-carboxylic acid 23

Please see Section 2.4

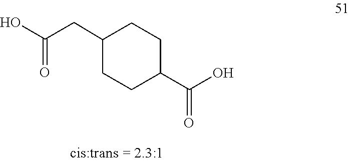 [SMILES: O=C(O)CC1CCC(C(=O)O)CC1]

### 4-(Carboxymethyl)cyclohexane-1-carboxylic acid 51

This compound was prepared according to the following procedure.(36) 4-(Carboxymethyl)benzoic acid (0.3 g, 1.67 mmol) was dissolved in glacial acetic acid (15.0 mL) and PtO₂ (60.0 mg) was added. The reaction vessel was purged with H₂ and the hydrogenation reaction was carried out at room temperature with a ballon of H₂ and stirred for 3 days. Analysis of an aliquot of the reaction mixture at this point suggested completion of reaction. The reaction mixture was filtered with a plug of Celite® and the filtrate was evaporated to dryness to give the titled compound as a white solid in 93% yield (0.29 g, 1.56 mmol).

NMR for minor diastereomer: ¹H NMR (500 MHz, DMSO) δ 2.09-2.05 (m, 3H), 1.89-1.79 (m, 2H), 1.79-1.69 (m, 2H), 1.59 (ddt, J=11.4, 7.7, 3.9 Hz, 1H), 1.28 (qd, J=13.1, 3.4 Hz, 2H), 0.96 (qd, J=13.0, 3.5 Hz, 2H).

¹³C NMR (126 MHz, DMSO) δ 176.7, 173.7, 42.3, 41.4, 33.7, 31.4, 28.5.

NMR for major diastereomer: ¹H NMR (500 MHz, DMSO) δ 2.43 (q, J=4.9 Hz, 1H), 2.10 (d, J=7.3 Hz, 2H), 1.89-1.79 (m, 2H), 1.79-1.69 (m, 1H), 1.55-1.50 (m, 2H), 1.47 (ddd, J=13.9, 8.7, 4.5 Hz, 2H), 1.18 (dtd, J=15.2, 11.0, 4.7 Hz, 2H).

¹³C NMR (126 MHz, DMSO) δ 176.1, 173.8, 39.9, 39.3, 32.4, 28.9, 25.7. HRMS (ESI-TOF) Calculated for C₉H₁₃O₄ [M−H]⁻: 185.0814,

Found: 185.0813.

### EXPERIMENTAL SECTION REFERENCES

1. R. Giri, N. Maugel, J. J. Li, D. H. Wang, S. P. Breazzano, L. B. Saunders, J. Q. Yu, Palladium-catalyzed methylation and arylation of sp² and sp³ C—H bonds in simple carboxylic acids. Journal of the American Chemical Society. 129, 3510-3511 (2007).
2. Z. Zhuang, C. B. Yu, G. Chen, Q. F. Wu, Y. Hsiao, C. L. Joe, J. X. Qiao, M. A. Poss, J. Q. Yu, Ligand-Enabled β-C(sp³)-H Olefination of Free Carboxylic Acids. Journal of the American Chemical Society. 140, 10363-10367 (2018).
3. Z. Zhuang, A. N. Herron, Z. Fan, J. Q. Yu, Ligand-Enabled Monoselective β-C(sp³)-H Acyloxylation of Free Carboxylic Acids Using a Practical Oxidant. Journal of the American Chemical Society. 142, 6769-6776 (2020).
4. Z. Zhuang, J. Q. Yu, Lactonization as a general route to β-C(sp³)-H functionalization. Nature. 577, 656-659 (2020).
5. F. Ghiringhelli, A. Uttry, K. K. Ghosh, M. van Gemmeren, Direct β-and γ-C(sp³)-H Alkynylation of Free Carboxylic Acids. Angewandte Chemie International Edition. 59, 23127-23131 (2020).
6. Z. Zhuang, A. N. Herron, J. Q. Yu, Synthesis of Cyclic Anhydrides via Ligand-Enabled C—H Carbonylation of Simple Aliphatic Acids. Angewandte Chemie—International Edition. 60, 16382-16387 (2021).
7. L.-C. Kao, A. Sen, Platinum(II) Catalysed Selective Remote Oxidation of Unactivated C—H Bonds in Aliphatic Carboxylic Acids. J. Chem. Soc., Chem. Commun., 1242-1243 (1991).
8. B. D. Dangel, J. A. Johnson, D. Sames, Selective functionalization of amino acids in water: A synthetic method via catalytic C—H bond activation. Journal of the American Chemical Society. 123, 8149-8150 (2001).
9. K. K. Ghosh, M. van Gemmeren, Pd-Catalyzed β-C(sp³)-H Arylation of Propionic Acid and Related Aliphatic Acids. Chemistry—A European Journal. 23, 17697-17700 (2017).
10. K. K. Ghosh, A. Uttry, A. Koldemir, M. Ong, M. van Gemmeren, Direct β-C(sp³)-H acetoxylation of aliphatic carboxylic acids. Organic Letters. 21, 7154-7157 (2019).
11. K. K. Ghosh, A. Uttry, F. Ghiringhelli, A. Mondal, M. van Gemmeren, Ligand-Enabled γ-C(sp³)-H Olefination of Free Carboxylic Acids. Angew. Chem. Int. Ed. 59, 12848-12852 (2020).
12. A. Uttry, S. Mal, M. van Gemmeren, Late-Stage β-C(sp³)-H Deuteration of Carboxylic Acids. Journal of the American Chemical Society. 143, 10895-10901 (2021).
13. Z. Wang, L. Hu, N. Chekshin, Z. Zhuang, S. Qian, J. X. Qiao, J.-Q. Yu, Ligand-controlled divergent dehydrogenative reactions of carboxylic acids via C—H activation. Science. 374, 1281-1285 (2021).
14. M. Cianfanelli, G. Olivo, M. Milan, R. J. M. Klein Gebbink, X. Ribas, M. Bietti, M. Costas, Enantioselective C—H Lactonization of Unactivated Methylenes Directed by Carboxylic Acids. Journal of the American Chemical Society. 142, 1584-1593 (2020).
15. L. Vicens, M. Bietti, M. Costas, General Access to Modified α-Amino Acids by Bioinspired Stereoselective γ-C—H Bond Lactonization. Angewandte Chemie—International Edition. 60, 4740-4746 (2021).
16. M. S. Chen, M. C. White, A Predictably Selective Aliphatic C—H Oxidation Reaction for Complex Molecule Synthesis. Science. 318, 783-787 (2007).
17. J. Schwarz, B. Konig, Decarboxylative reactions with and without light—a comparison. Green Chemistry. 20, 323-361 (2018).
18. S. K. Sartori, M. A. N. Diaz, G. Diaz-Munoz, Lactones: Classification, synthesis, biological activities, and industrial applications. Tetrahedron. 84, 132001-132039 (2021).
19. M. Seitz, O. Reiser, Synthetic approaches towards structurally diverse γ-butyrolactone natural-product-like compounds. Current Opinion in Chemical Biology. 9 (2005), pp. 285-292.
20. H. T. Simonsen, C. Weitzel, S. B. Christensen, in Natural Products: Phytochemistry, Botany and Metabolism of Alkaloids, Phenolics and Terpenes (Springer Berlin Heidelberg, 2013), pp. 3069-3098.
21. K. Strømgaard, K. Nakanishi, Chemistry and biology of terpene trilactones from Ginkgo biloba. Angewandte Chemie—International Edition. 43, 1640-1658 (2004).
22. G. Shabir, A. Saeed, H. R. El-Seedi, Natural isocoumarins: Structural styles and biological activities, the revelations carry on. Phytochemistry. 181, 112568-112787 (2021).
23. A. D. Kinghorn, H. Falk, S. Gibbons, J. Kobayashi, Eds., Progress in the Chemistry of Organic Natural Products (Springer Nature, 2017), vol. 104.
24. F. Zaragoza Dörwald, Side Reactions in Organic Synthesis: A Guide to Successful Synthesis Design (Wiley, 2004).
25. G. Xia, J. Weng, L. Liu, P. Verma, Z. Li, J. Q. Yu, Reversing conventional site-selectivity in C(sp 3)-H bond activation. Nature Chemistry. 11, 571-577 (2019).
26. P. Novak, A. Correa, J. Gallardo-Donaire, R. Martin, Synergistic palladium-catalyzed C(sp³)-H Activation/C(sp³)-O bond formation: A direct, step-economical route to benzolactones. Angewandte Chemie—International Edition. 50, 12236-12239 (2011).
27. J. M. Lee, S. Chang, Pt-Catalyzed sp³ C—H bond activation of o-alkyl substituted aromatic carboxylic acid derivatives for the formation of aryl lactones. Tetrahedron Letters. 47, 1375-1379 (2006).
28. T. Lin, G. Wang, W. Shan, D. Zeng, R. Ding, X. Jiang, D. Zhu, X. Liu, S. Yang, H. Chen, Myrotheciumones: Bicyclic cytotoxic lactones isolated from an endophytic fungus of Ajuga decumbens. Bioorganic and Medicinal Chemistry Letters. 24, 2504-2507 (2014).
29. S. N. Khan, M. K. Zaman, R. Li, Z. Sun, A General Method for Photocatalytic Decarboxylative Hydroxylation of Carboxylic Acids. Journal of Organic Chemistry. 85, 5019-5026 (2020).
30. S. Begum, T. K. Chakraborty, Cp2TiCl-Mediated Reductive Cyclization: Total Synthesis of Pestalotiolactone A, Myrotheciumone A, and Scabrol A. Journal of Organic Chemistry. 86, 11812-11821 (2021).
31. E. M. Mpondo, J. Garcia, A. J. Chulia, New Phthalides from Gentiana pedicellata. Planta Medica. 53, 297-298 (1987).
32. Z. Li, Z. Wang, N. Chekshin, S. Qian, J. X. Qiao, P. T. Cheng, K.-S. Yeung, W. R. Ewing, J.-Q. Yu, A tautomeric ligand enables directed C—H hydroxylation with molecular oxygen. Science. 372, 1452-1457 (2021).
33. O. S. Shneider, E. Pisarevsky, P. Fristrup, A. M. Szpilman, Oxidative umpolung α-alkylation of ketones. Organic Letters. 17, 282-285 (2015).
34. M. Azimioara, P. Alper, C. Cow, D. Mutnick, V. Nikulin, G. Lelais, J. Mecom, M. McNeill, P. Y. Michellys, Z. Wang, E. Reding, M. Paliotti, J. Li, D. Bao, J. Zoll, Y. Kim, M. Zimmerman, T. Groessl, T. Tuntland, S. B. Joseph, P. McNamara, H. M. Seidel, R. Epple, Novel tricyclic pyrazolopyrimidines as potent and selective GPR119 agonists. Bioorganic and Medicinal Chemistry Letters. 24, 5478-5483 (2014).
35. D. D. Bume, C. R. Pitts, R. T. Jokhai, T. Lectka, Direct, visible light-sensitized benzylic C—H fluorination of peptides using dibenzosuberenone: selectivity for phenylalanine-like residues. Tetrahedron. 72, 6031-6036 (2016).
36. J. T. Pinhey, E. Ritchie, W. C. Taylor, The Chemical Constituents Of Himantandra (Galbulimima) Species. Australian Journal of Chemistry. 14, 106-134 (1961).
37. J. le Nôtre, D. van Mele, C. G. Frost, A new method for constructing quaternary carbon centres: Tandem rhodium-catalysed 1,4-addition/intramolecular cyclisation. Advanced Synthesis and Catalysis. 349, 432-440 (2007).
38. F. Bonati, G. Minghetti, Mercury(II) Derivatives Of Acetylacetone. Journal of Organometallic Chemistry. 22, 5-10 (1970).

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A method of γ- or δ-lactonization via β-C—H activation, comprising i) treating a dicarboxylic acid substrate with a quinoline-pyridone or pyridine-pyridone ligand in the presence of a Pd source; and ii) addition of p-xyloquinone (BQ3), an Ag salt, and K₂HPO₄ in a reaction vessel.

2. The method of claim 1, wherein the quinoline-pyridone or pyridine-pyridone ligand is selected from the group consisting of:

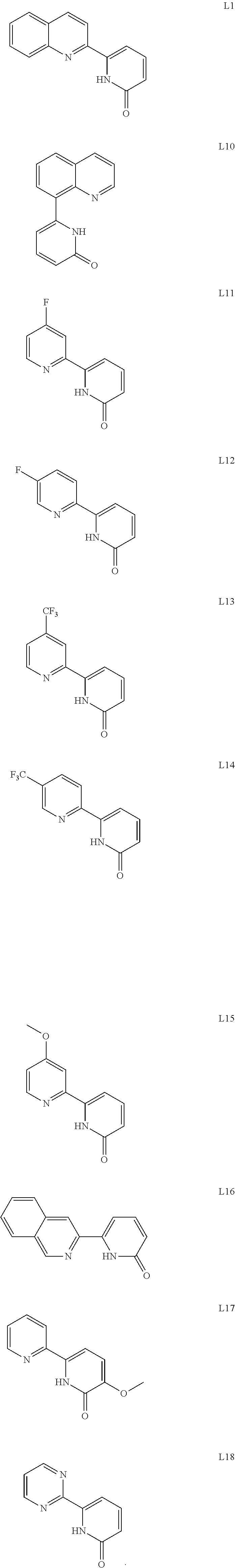

3. The method of any one of claims 1-2, wherein the Pd source is Pd(OAc)₂.

4. The method of any one of claims 1-3, wherein the Ag salt is Ag₂CO₃.

5. The method of any one of claims 1-4, wherein the quinoline-pyridone ligand is L1.

6. The method of any one of claims 1-4, wherein the pyridine-pyridone ligand is L18.

7. The method of any one of claims 1-4, wherein the pyridine-pyridone ligand is L17.

8. The method of any one of claims 1-4, wherein the pyridine-pyridone ligand is L13.

9. The method of any one of claims 1-4, wherein the pyridine-pyridone ligand is L14.

10. The method of any one of claims 1-4, wherein the pyridine-pyridone ligand is L11.

11. The method of any one of claims 1-4, wherein the quinoline-pyridone ligand is L10.

12. The method of any one of claims 1-4, wherein the quinoline-pyridone ligand is L16.

13. The method of any one of claims 1-4, wherein the pyridine-pyridone ligand is L12.

14. The method of any one of claims 1-4, wherein the pyridine-pyridone ligand is L15.

15. The method of γ-C—H lactonization via β-C—H activation of claim 1, wherein the dicarboxylic acid substrate is 1.0 eq. adipic acid, the quinoline-pyridone ligand is 12 mol % L1, the Pd source is 10 mol % Pd(OAc)₂, the Ag salt is 2.0 eq. Ag₂CO₃, with 2.0 eq. BQ3, 1.0 eq. K₂HPO₄ in HFIP at 100° C. for 36 h and the reaction vessel is an 8-10 mL vial.

16. The method of δ-lactonization via —C—H activation of claim 1, wherein the dicarboxylic acid substrate is 1.0 eq. pimelic acid, the quinoline-pyridone ligand is 12 mol % L1, the Pd source is 10 mol % Pd(OAc)₂, the Ag salt is 2.0 eq. Ag₂CO₃, with 2.0 eq. BQ3, 1.0 eq. K₂HPO₄ in HFIP at 100° C. for 36 h and the reaction vessel is an 8-10 mL vial.

17. The method of either one of claim 15 or 16, wherein the 1.0 eq. K₂HPO₄ is replaced with 0.75 eq. K₂HPO₄.

18. The method of either one of claim 15 or 16, wherein the 1.0 eq. K₂HPO₄ is replaced with a mixture of 0.35 eq. K₂HPO₄ and 0.4 eq. CsOAc.

19. The method of either one of claim 15 or 16, wherein the 2.0 eq. Ag₂CO₃ is replaced with 4.0 eq. MnO₂ and the 1.0 eq. K₂HPO₄ is replaced with K₂HPO₄:KH₂PO₄:CsOAc (1.0:1.5:1.0, 0.75 eq. total).

20. A method of γ-lactonization via γ-C—H activation comprising i) treating a dicarboxylic acid substrate with a quinoline-pyridone or pyridine-pyridone ligand in the presence of a Pd source; and ii) addition of BQ3, Ag₂CO₃ and K₂HPO₄ in a reaction vessel.

21. The method of claim 20, wherein the quinoline-pyridone or pyridine-pyridone ligand is selected from the group consisting of:

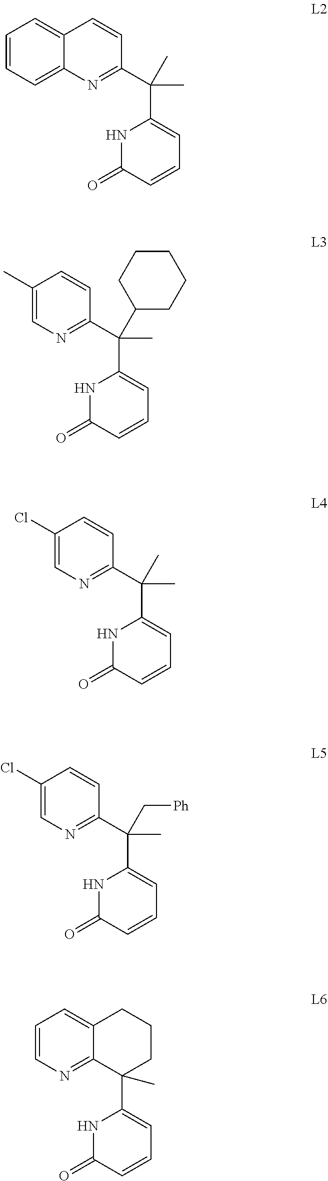

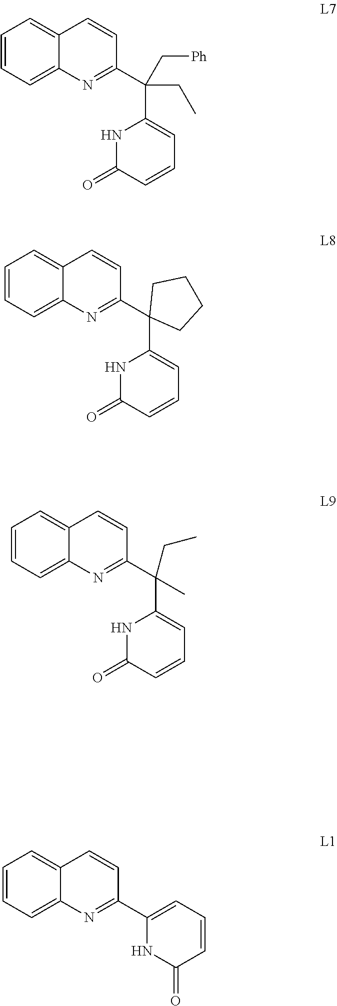 [SMILES: CCC(Cc1ccccc1)(c1ccc2ccccc2n1)c1cccc(=O)[nH]1 ; O=c1cccc(C2(c3ccc4ccccc4n3)CCCC2)[nH]1 ; CCC(C)(c1ccc2ccccc2n1)c1cccc(=O)[nH]1 ; O=c1cccc(-c2ccc3ccccc3n2)[nH]1]

22. The method of claim 21, wherein the quinoline-pyridone ligand is L2.

23. The method of claim 21, wherein the quinoline-pyridone ligand is L9.

24. The method of claim 21, wherein the quinoline-pyridone ligand is L8.

25. The method of claim 21, wherein the quinoline-pyridone ligand is L7.

26. The method of claim 21, wherein the pyridine-pyridone ligand is L5.

27. The method of claim 21, wherein the pyridine-pyridone ligand is L4.

28. The method of claim 21, wherein the quinoline-pyridone ligand is L6.

29. The method of claim 21, wherein the quinoline-pyridone ligand is L1.

30. A method of γ-lactonization via γ-C—H activation comprising i) treating a dicarboxylic acid substrate with L2 in the presence of a Pd source; and ii) addition of an oxidant and K₂HPO₄.

31. The method of claim 30, wherein the oxidant is 2.0 eq. Na₂S₂O₈.

32. The method of claim 30, wherein the oxidant is 2.0 eq. K₂S₂O₈.

33. The method of claim 30, wherein the oxidant is 2.0 eq. BzOO^{t}Bu.

34. The method of claim 30, wherein the oxidant is 2.0 eq. AcOO^{t}Bu.

35. The method of claim 30, wherein the oxidant is 2.0 eq. Ce(SO₄)₂.

36. The method of claim 30, wherein the oxidant is 2.0 eq. CMHP.

37. The method of claim 30, wherein the oxidant is 2.0 eq. ^{t}BuOO^{t}Bu.

38. The method of claim 30, wherein the oxidant is 2.0 eq. 1-iodo-3,5-bis(trifluoromethyl)benzene.

39. The method of claim 30, wherein the oxidant is 2.0 eq. methyl 4-iodobenzoate.

40. The method of claim 30, wherein the oxidant is 2.0 eq. 1,2,3,4,5-pentafluoro-6-iodobenzene.

41. The method of claim 30, wherein the oxidant is 2.0 eq. TBHP in H₂O.

42. The method of claim 30, wherein the oxidant is 2.0 eq. H₂O₂ in H₂O.

43. The method of claim 30, wherein the oxidant is selected from the group consisting of:

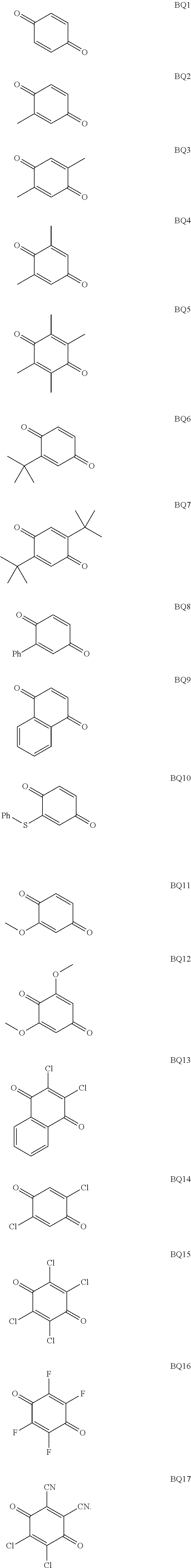

44. The method of claim 43, wherein the oxidant is BQ8.

45. The method of claim 43, wherein the oxidant is BQ5.

46. The method of claim 43, wherein the oxidant is BQ4.

47. The method of claim 43, wherein the oxidant is BQ6.

48. The method of claim 43, wherein the oxidant is BQ1.

49. The method of claim 43, wherein the oxidant is BQ12.

50. The method of claim 43, wherein the oxidant is BQ2.

51. The method of claim 43, wherein the oxidant is BQ9.

52. The method of claim 43, wherein the oxidant is BQ7.

53. The method of claim 43, wherein the oxidant is BQ11.

54. The method of claim 43, wherein the oxidant is BQ13.

55. The method of claim 30, wherein the oxidant is AgOAc.

56. The method of claim 30, wherein the oxidant is Ag2O.

57. The method of claim 30, wherein the oxidant is AgF.

58. The method of claim 30, wherein the oxidant is Ag₂CO₃.

59. The method of claim 30, wherein the oxidant is AgNO₃.

60. The method of claim 30, wherein the oxidant is Ag₃PO₄.

61. The method of claim 30, wherein the oxidant is CuSO₄·5H₂O.

62. The method of claim 30, wherein the oxidant is CuF₂.

63. The method of claim 30, wherein the oxidant is CuO.

64. The method of claim 30, wherein the oxidant is Cu₃(PO₄)₂.

65. The method of claim 30, wherein the oxidant is CuBr₂.

66. The method of claim 30, wherein the oxidant is CuCO₃.

67. The method of claim 30, wherein the oxidant is a mixture of BQ3 and an Ag salt.

68. The method of claim 67, wherein the Ag salt is Ag₂CO₃.

69. The method of claim 67, wherein the Ag salt is Ag₃PO₄.

70. The method of claim 67, wherein the Ag salt is AgF.

71. The method of claim 67, wherein the Ag salt is Ag₂O.

72. The method of claim 67, wherein the Ag salt is AgOAc.

73. The method of any one of claims 20-72, wherein the Pd source is Pd(OAc)₂.

74. The method of any one of claims 20-72, wherein the Pd source is Pd(TFA)₂.

75. The method of any one of claims 20-72, wherein the Pd source is Pd(MeCN)₄(BF₄)₂.

76. The method of any one of claims 20-72, wherein the Pd source is Pd(MeCN)₄(OTf)₂.

77. The method of any one of claims 20-72, wherein the Pd source is PdCl₂.

78. The method of any one of claims 20-72, wherein the Pd source is PdCl₂(PhCN)₂.

79. The method of any one of claims 20-72, wherein the Pd source is Pd₂(dba)₃.

80. The method of any one of claims 20-72, wherein the Pd source is PdCl₂(MeCN)₂.

81. The method of any one of claims 20-80, wherein the dicarboxylic acid substrate is pimelic acid

82. The method of any one of claims 20-80, wherein the dicarboxylic acid substrate is 2,2,6,6-tetramethylpimelic acid.

83. The method of claim 20, wherein the dicarboxylic acid substrate is 1.0 eq. pimelic acid, the quinoline-pyridone ligand is 12 mol % L2, the Pd source is 10 mol % Pd(OAc)₂, the oxidant is 2.0 eq. BQ3 and 2.0 eq. of an Ag salt, and 1.0 eq. K₂HPO₄, the reaction vessel is a vial between 8-10 mL, the reaction temperature is between 80-100° C., and the reaction time is between 12-72 h.

84. The method of claim 83, wherein the Ag salt is Ag₂CO₃.

85. The method of claim 83, wherein the Ag salt is Ag₃PO₄.

86. The method of claim 83, wherein the Ag salt is AgF.

87. The method of claim 83, wherein the Ag salt is Ag₂O.

88. The method of claim 83, wherein the Ag salt is AgOAc.

89. The method of claim 84, wherein the Ag₂CO₃ is replaced by K₂S₂O₈.

90. The method of claim 84, wherein the Ag₂CO₃ is replaced with 0.5 eq. Ag₂CO₃ in addition to replacing the 2.0 eq. BQ3 with 0.5 eq. BQ3.

91. The method of claim 84, wherein the 2.0 eq. Ag₂CO₃ is replaced with 1.0 eq. Ag₂CO₃ in addition to replacing the 2.0 eq. BQ3 with 1.0 eq. BQ3.

92. The method of any one of claims 20-91, wherein the reaction temperature is 100° C.

93. The method of any one of claims 20-91, wherein the reaction temperature is 80° C.

94. The method of any one of claims 20-91, wherein the reaction temperature is 120° C.

95. The method of any one of claims 20-94, wherein the reaction time is 12 h.

96. The method of any one of claims 20-94, wherein the reaction time is 24 h.

97. The method of any one of claims 20-94, wherein the reaction time is 36 h.

98. The method of any one of claims 20-94, wherein the reaction time is 72 h.

99. The method of any one of claims 20-98, wherein the reaction vessel is a 10 mL vial.

100. The method of any one of claims 20-98, wherein the reaction vessel is an 8 mL vial, the reaction temperature is 100° C., and the reaction time is 36 h.

101. The method of claim 83, wherein the 1.0 eq. of K₂HPO₄ is replaced with 0.75 eq. K₂HPO₄ and the reaction vessel is an 8 mL vial, the reaction temperature is 100° C., and the reaction time is 36 h.

102. The method of claim 83, wherein the 1.0 eq. of K₂HPO₄ is replaced with 0.35 eq K₂HPO₄ and 0.4 eq. CsOAc, the reaction vessel is an 8 mL vial, the reaction temperature is 100° C., and the reaction time is 36 h.

103. The method of claim 83, wherein the 1.0 eq. of K₂HPO₄ is replaced with 0.35 eq K₂HPO₄ and 0.4 eq. CsOAc, L2 is replaced with L10, and the reaction vessel is an 8 mL vial, the reaction temperature is 100° C., and the reaction time is 36 h.

104. The method of claim 83, wherein the 1.0 eq. of K₂HPO₄ is replaced with 0.35 eq K₂HPO₄ and 0.4 eq. CsOAc, L2 is replaced with L10, the reaction vessel is an 8 mL vial, the reaction temperature is 100° C., and the reaction time is 48 h.

105. The method of claim 83, wherein the 2.0 eq. of an Ag salt is replaced with 2.0 eq. of MnO₂.

106. The method of claim 83, wherein the 2.0 eq. of an Ag salt is replaced with 6.0 eq. of MnO₂.

107. The method of claim 83, wherein the 2.0 eq. of an Ag salt is replaced with K₂HPO₄:KH₂PO₄:CsOAc (1:1.5:1, total 0.75 eq.).

108. The method of claim 83, wherein the 2.0 eq. of an Ag salt is replaced with K₂HPO₄:KH₂PO₄:CsOAc (1:1.5:1, total 1.0 eq.).

109. The method of claim 83, wherein the 2.0 eq. of an Ag salt is replaced with K₂HPO₄:KH₂PO₄:CsOAc (1:2:1, total 0.75 eq.).

110. The method of claim 83, wherein the 2.0 eq. of an Ag salt is replaced with K₂HPO₄:KH₂PO₄:CsOAc (1:2:1, total 1.0 eq.).

111. The method of claim 83, wherein the reaction time is 48 h.

112. The method of claim 83, wherein the 2.0 eq. of an Ag salt is replaced with K₂HPO₄:KH₂PO₄:CsOAc (1:1.5:1, total 0.75 eq.) and L2 is replaced with L10.

113. The method of claim 83, wherein the 2.0 eq. of an Ag salt is replaced with K₂HPO₄:KH₂PO₄:CsOAc (1:1.5:1, total 0.75 eq.), L2 is replaced with L10, and the reaction time is 48 h.

114. The method of claim 83, wherein, the oxidant is 2.0 eq. BQ3 and 2.0 eq. of Ag₂CO₃, the reaction vessel is a 10 mL vial, the reaction temperature is 100° C., and the reaction time is 36 h.

115. The method of claim 83, comprising i) treating 1.0 eq. pimelic acid with 12 mol % ligand L2 in the presence of 10 mol % Pd(OAc)₂; and ii) addition of 2.0 eq. Ag₂CO₃, 2.0 eq. BQ3, and 1.0 eq. K₂HPO₄ at 100° C. in HFIP for 36 h in an 8 mL vial.

116. A method for the total synthesis of Myrotheciumone A. comprising the following steps:
(1) reaction of MeI (2.0 eq.) and K₂CO₃ (3.0 eq.), in acetone, and refluxed for 3 hr;
(2) reaction with Ph₃PCH₃Br (2.9 eq.) and tBuOK (2.4 eq.), in toluene, at r.t. overnight;
(3) reaction with mCPBA (1.2 eq.), in CH₂Cl₂, at r.t. overnight;
(4) reaction with TMSOTf (2.0 eq.) and 2,6-lutidine (2.0 eq.), in toluene, at −78° C. to r.t. overnight;
(5) reaction with cat. Pivalic acid (10 mol %) and triethyl orthoacetate, neat, at 155° C., overnight and further reaction with cat. p-TsOH (10 mol %), in toluene, and refluxed overnight;
(6) reaction with cat. PtO₂, under H₂ (4-layered ballon), in AcOH, at r.t. overnight;
(7) reaction with 15% aq. NaOH, and refluxed overnight;
(8) reaction with Pd(OAc)₂ (10 mol %), Ligand L2 (12 mol %), BQ3 (2.0 eq.), Ag₂CO₃ (2.0 eq.), K₂HPO₄ (0.35 eq.), and CsOAc (0.40 eq.), in HFIP, at 100° C., for 36 h; and
(9) reaction with (Ir[dF(CF₃)ppy]₂(dtbpy))PF₆ (1 mol %), Cs₂CO₃ (1.5 eq.), and NaBH₄ (1.2 eq.), under 02 atmosphere, in CH₂Cl₂, at 40° C., 4×100 W Blue LED lamps, for 40 h.

117. A method for the total synthesis of Pedicellosine comprising the following steps:
(1) reaction of Pd(OAc)₂ (10 mol %), Ligand L2 (12 mol %), BQ3 (2.0 eq.), Ag₂CO₃ (2.0 eq.), K₂HPO₄ (0.35 eq.), and CsOAc (0.40 eq.), in HFIP, at 100° C., for 36 h;
(2) reaction with BH₃·Me₂S (1.6 eq.), in THF, at 0° C. to r.t. overnight; and
(3) reaction with EDCI (1.5 eq.), DMAP (20 mol %), and 2,3-dihydroxybenzoic acid (1.5 eq.), in CH₂Cl₂, at r.t. overnight.

118. Any method of γ-lactonization via β-C—H activation, δ-lactonization via β-C—H activation, γ-lactonization via γ-C—H activation, or total syntheses of natural products as disclosed herein.
